(19) United States
(12) Patent Application Publication
(10) Pub. No.: US 2026/0035406 A1
(43) Pub. Date: Feb. 5, 2026

(54) SYNTHESIS OF GLP-1R/GIPR AGONISTS

(71) Applicants: Carmot Therapeutics Inc., South San Francisco, CA (US); Hoffmann-La Roche Inc., Little Falls, NJ (US)

(72) Inventors: Shyam Krishnan, Novato, CA (US); Chet Sodkomkum, Pasadena, CA (US); Matteo Villain, Rancho Palos Verdes, CA (US); Christopher E. Martin, Redondo Beach, CA (US); Markus Wiesner, Bubendorf (CH); Ralph Oscar Schönleber, Bubendorf (CH); Phil Servatius, Bubendorf (CH); Gàbor Norbert Boross, Bubendorf (CH); Jan Paul Hoyer, Bubendorf (CH); Pascal Engi, Bubendorf (CH); Niall McLoughlin, Basel (DE)

(73) Assignees: Carmot Therapeutics Inc., South San Francisco, CA (US); Hoffmann-La Roche Inc., Little Falls, NJ (US)

(21) Appl. No.: 19/287,729

(22) Filed: Jul. 31, 2025

Related U.S. Application Data
(60) Provisional application No. 63/678,044, filed on Jul. 31, 2024.
(60) Provisional application No. 63/837,108, filed on Jul. 1, 2025.

(51) Int. Cl. C07K 1/22; C07K 1/02; C07K 1/04; C07K 1/06; C07K 1/18

(52) U.S. Cl. CPC C07K 1/22; C07K 1/02; C07K 1/042; C07K 1/061; C07K 1/18

(57) ABSTRACT

The present disclosure provides novel synthetic methods for preparing GLP-1R/GIPR agonists.

### CROSS-REFERENCE TO RELATED APPLICATIONS

The present application claims priority from U.S. Applications 63/678,044, filed Jul. 31, 2024 and 63/837,108, filed Jul. 1, 2025. The contents of the priority applications are incorporated by reference herein in their entirety.

### SEQUENCE LISTING

The instant application contains a Sequence Listing that has been submitted electronically in XML format and is hereby incorporated by reference in its entirety. Said XML copy, created on Jul. 31, 2025, is named 124921_US010_SL.xml and is 60,465 bytes in size.

### BACKGROUND OF THE INVENTION

Incretin hormones are hormones that provide glycemic control in response to food intake. Gastric inhibitory polypeptide (“GIP”) and glucagon-like peptide-I (“GLP-1”) are primary incretin hormones secreted from small intestinal L cells and K cells, respectively, on ingestion of glucose or nutrients to stimulate insulin secretion from pancreatic cells. GIP and GLP-1 undergo degradation by dipeptidyl peptidase-4 (DPP-4), and rapidly lose their biological activities (see, e.g., Sieno et al., J Diab Invest. (2013) 4:108-30).

The actions of GIP and GLP-1 are believed to be mediated by their receptors, the GIP receptor (GIPR) and the GLP-1 receptor (GLP-1R), respectively, which both belong to the G-protein coupled receptor family and are expressed in pancreatic cells, as well as in various tissues and organs. GLP-1 activities include, without limitation, stimulation of insulin synthesis and secretion, inhibition of glucagon secretion, and inhibition of food intake. GIP activities include, without limitation, stimulation of glucose-dependent insulin secretion, an increase in cell mass, stimulation of glucagon secretion, and a decrease in gastric acid secretion.

GLP-1 and GLP-1 analogs, acting as agonists of GLP-IR, have been shown to be effective in glycemic control, e.g., in type-2 diabetes. See, e.g., WO 2016/131893. In addition to their insulinotropic effects, GIP and GLP-1 are believed to be involved in various biological processes in different tissues and organs that express GIPR and GLP-IR. Investigations using mice lacking GIPR and/or GLP-1R, as well as mice lacking DPP-4, showed involvement of GIP and GLP-1 in divergent biological activities. The results of these investigations point to involvement of GIP and GLP-1 in treating and/or preventing diabetes-related microvascular complications (e.g., retinopathy, nephropathy and neuropathy) and macrovascular complications (e.g., coronary artery disease, peripheral artery disease and cerebrovascular disease), as well as diabetes-related comorbidity (e.g., obesity, non-alcoholic fatty liver disease, bone fracture and cognitive dysfunction). See, e.g., Sieno et al., J Diab Invest. (2013) 4:108-30.

There remains a need for chemical entities that modulate the activity of GLP-1R and/or GIPR and for improved methods of synthesizing such chemical entities.

### SUMMARY OF THE INVENTION

The present disclosure provides a method of synthesizing an N-terminal conjugated peptidyl compound of formula (I):

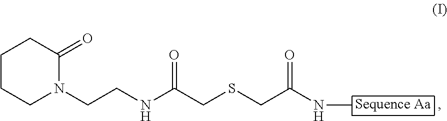 [SMILES: CNC(=O)CSCC(=O)NCCN1CCCCC1=O]

wherein Sequence Aa is a peptide, the method comprising the steps of (i) treating a resin-bound peptide of formula (II):

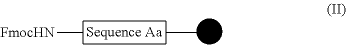

with 10 to 30%, e.g. 20%, piperidine in DMF, and (ii) reacting the product of step (i) with 2-((2-oxo-2-((2-(2-oxopiperidin-1-yl)ethyl)amino)ethyl)thio)acetic acid (III):

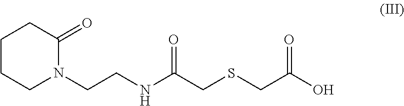 [SMILES: O=C(O)CSCC(=O)NCCN1CCCCC1=O]

under amide bond-forming conditions, wherein the amide bond-forming conditions comprise use of 2-(1H-Benzotriazole-1-yl)-1,1,3,3-tetramethylaminium tetrafluoroborate (TBTU).

Also provided is a method of synthesizing an N-terminal conjugated peptidyl compound of formula (I):

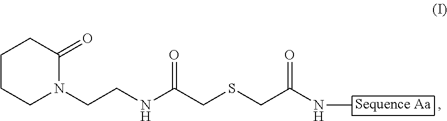 [SMILES: CNC(=O)CSCC(=O)NCCN1CCCCC1=O]

wherein Sequence Aa is a peptide, the method comprising the steps of (i) treating a resin-bound peptide of formula (II):

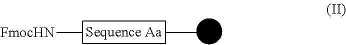

with 10 to 30%, e.g. 20%, piperidine in DMF, and (ii) reacting the product of step (i) with 2-((2-oxo-2-((2-(2-oxopiperidin-1-yl)ethyl)amino)ethyl)thio)acetic acid (III):

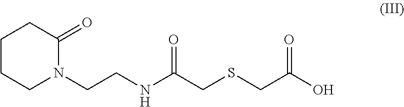 [SMILES: O=C(O)CSCC(=O)NCCN1CCCCC1=O]

under amide bond-forming conditions, wherein the amide bond-forming conditions comprise use of the combination of ethyl cyano (hydroxyimino) acetate (OxymaPure™) and N,N′-diisopropylcarbodiimide (DIC). The method may also further comprise cleaving the resin-bound peptide of formula (XXXII) from the resin:

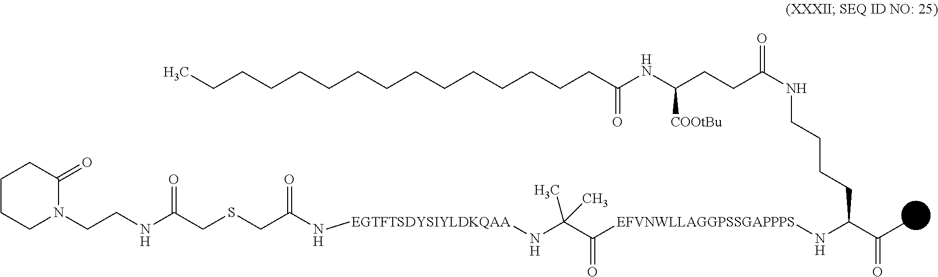

with a cleavage cocktail, optionally wherein the cleavage cocktail comprises trifluoroacetic acid (TFA) and a scavenger.

In some embodiments, Sequence Aa comprises the formula W—R⁵, wherein W is a peptide sequence and R⁵ is conjugated to the C-terminus of W, wherein R⁵ is a C-terminal amino acid amide or a C-terminal amino acid that is optionally substituted with 1 or 2 modifying groups selected from an acyl group and a PEG group, and wherein W comprises the following sequence: EGT(Xaa4)(Xaa5)SD(Xaa8)S(Xaa10)(Xaa11)(Xaa12)(Xaa13)(Xaa14)(Xaa15)(Xaa16)(Xaa17)(Xaa18)(Xaa19)(Xaa20)(Xaa21)(Xaa22)WL(Xaa25)(Xaa26)(Xaa27)GPSSGAPPP(Xaa37)(SEQ ID NO:1); wherein: Xaa4 is F; Xaa5 is T or I; Xaa8 is Y, V, L, or K*; Xaa10 is I or S; Xaa11 is Y, Y*, Q, A, or (Aib); Xaa12 is L, M, or L*; Xaa13 is D or E; Xaa14 is K, G, R, or E; Xaa15 is Q or I; Xaa16 is A, H, or R; Xaa17 is A, Q, or V; Xaa18 is A, (Aib), K*, K, or Q; Xaa19 is A, D, E, (Aib), or L; Xaa20 is F or A; Xaa21 is V or I; Xaa22 is N, A, Q, K*, or E; Xaa25 is I, L or V; Xaa26 is A, K, or I; Xaa27 is Q-R, G-R-G-K* (SEQ ID NO: 24), Q, or G; and Xaa37 is S or absent.

In some embodiments, W comprises the following sequence:

(SEQ ID NO: 2)
EGTFTSDYSIYLDKQAA(Aib)EFVNWLLAGGPSSGAPPPS.

R⁵ may be a C-terminal lysyl amide residue that is optionally substituted with 1 or 2 modifying groups selected from an acyl group and a PEG group. In some embodiments, R⁵ comprises formula (IV):

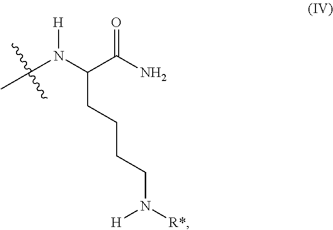

and R* comprises the structure (V):

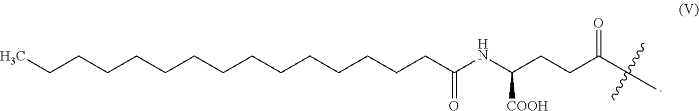 [SMILES: *C(=O)CC[C@H](NC(=O)CCCCCCCCCCCCCCC)C(=O)O]

In some embodiments, W—R⁵ comprises the structure (V):

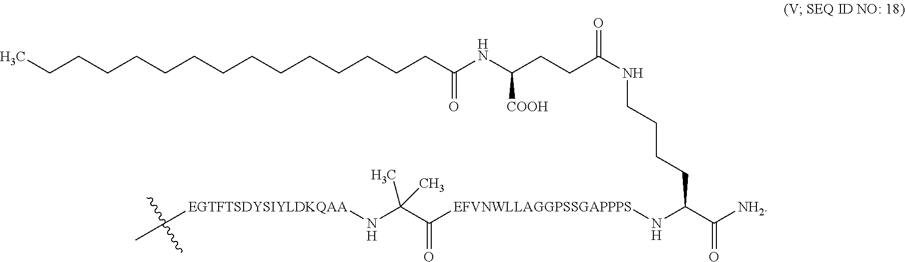

Also provided is a method of synthesizing a compound of formula (VI):

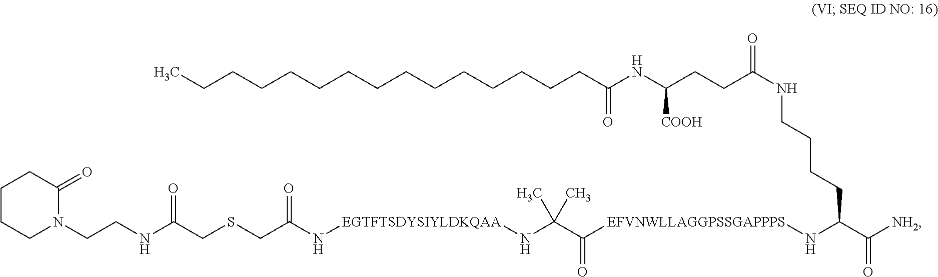

the method comprising the steps of (i) treating a resin-bound peptide of formula (VII):

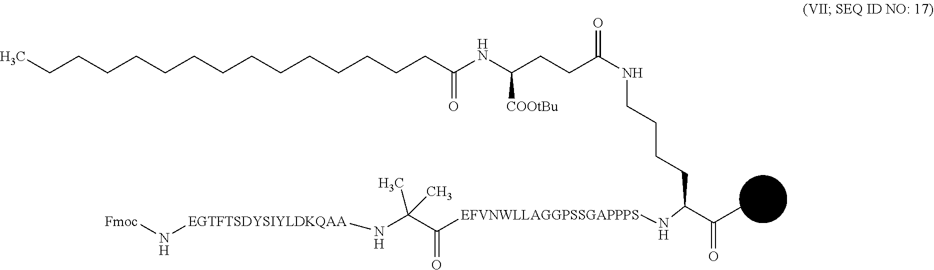

with 10 to 30%, e.g. 20%, piperidine in DMF, and (ii) reacting the product of step (i) with 2-((2-oxo-2-((2-(2-oxopiperidin-1-yl)ethyl)amino)ethyl)thio)acetic acid (III):

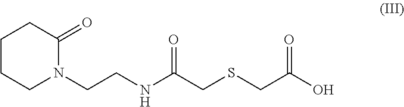 [SMILES: O=C(O)CSCC(=O)NCCN1CCCCC1=O]

under amide bond-forming conditions, wherein the amide bond-forming conditions comprise use of 2-(1H-Benzotriazole-1-yl)-1,1,3,3-tetramethylaminium tetrafluoroborate (TBTU). The method may further comprise cleaving the peptide from the resin, wherein the cleaving comprises treating the resin-bound peptide of formula (XXXII):

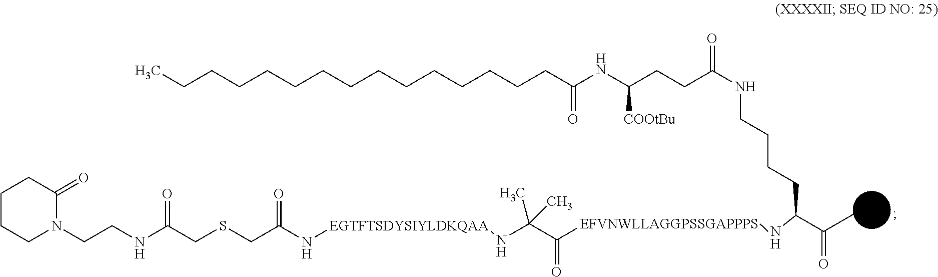

with a cleavage cocktail, optionally wherein the cleavage cocktail comprises trifluoroacetic acid (TFA) and a scavenger.

Also provided is a method of synthesizing a compound of formula (VI):

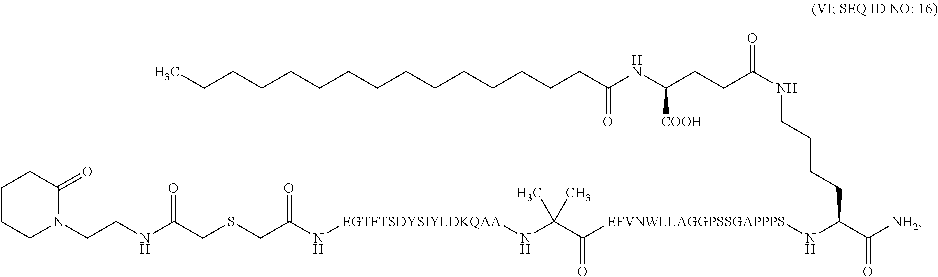

the method comprising the steps of (i) treating a resin-bound peptide of formula (VII):

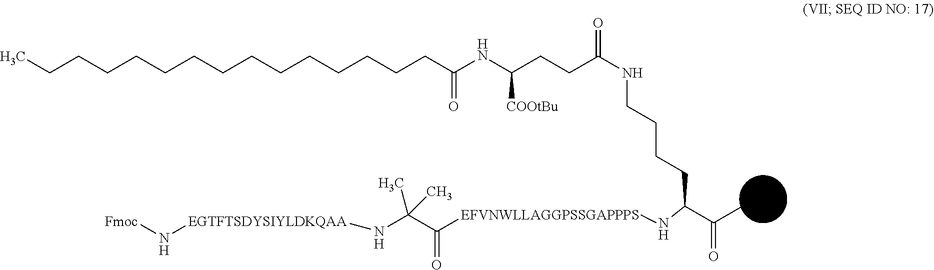

with 10 to 30%, e.g. 20%, piperidine in DMF, and (ii) reacting the product of step (i) with 2-((2-oxo-2-((2-(2-oxopiperidin-1-yl)ethyl)amino)ethyl)thio)acetic acid (III):

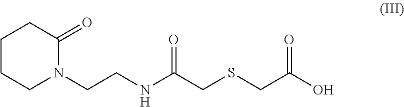 [SMILES: O=C(O)CSCC(=O)NCCN1CCCCC1=O]

under amide bond-forming conditions, wherein the amide bond-forming conditions comprise use of the combination of ethyl cyano (hydroxyimino) acetate (e.g., OxymaPure™) and N,N′-diisopropylcarbodiimide (DIC). The method may further comprise the steps of cleaving the peptide from the resin, wherein the cleaving comprises treating the resin-bound peptide of formula (XXXII):

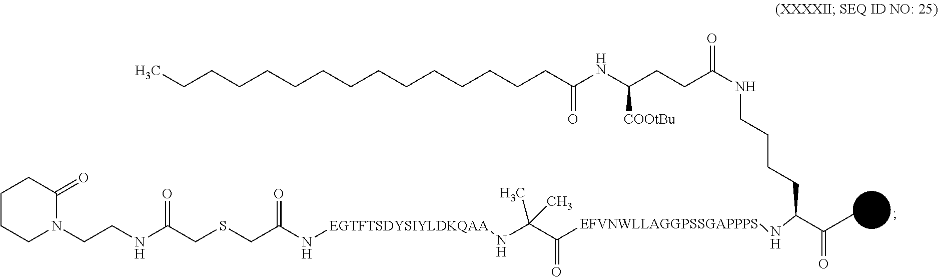

with a cleavage cocktail, optionally wherein the cleavage cocktail comprises trifluoroacetic acid (TFA) and a scavenger.

In a particular embodiment, the N-terminal conjugated peptidyl compound of formula (I) is a GLP-1R/GIPR agonist having the structural formula (VI):

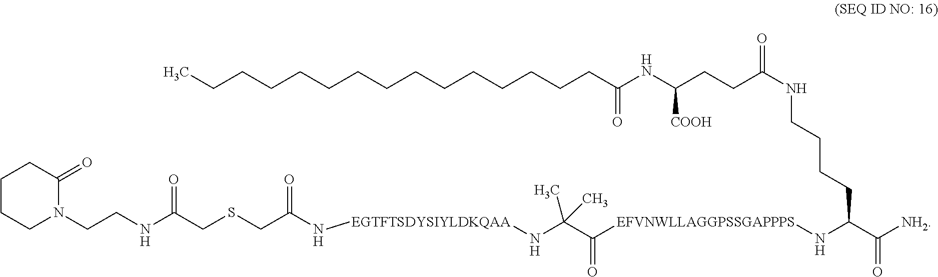

The dual GLP-1R/GIPR agonist is hereinafter also designated as CT-868.

In some embodiments, the resin is tricyclic amide linker resin. The method may comprise coupling Fmoc-Pro-Pro-OH, Fmoc-Ser(tBu)-Gly-OH, Fmoc-Gly-Gly-OH, Fmoc-Thr(tBu)-Ser(Ψ(Me,Me) pro)-OH, Fmoc-Asp(OMpe)-OH, Fmoc-Lys(Palmitoyl-Glu-OtBu)-OH, Fmoc-Gly-Pro-OH, Fmoc-Ala-Gly-OH, Fmoc-Ala-Pro-OH, Fmoc-Gly-Thr(ΨMe,Mepro)-OH or Fmoc-Ala-Aib-OH to the resin-bound peptide.

In some embodiments, the method comprises the steps of (a) treating a resin-bound amine of formula (VIII):

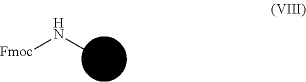

with 10 to 30%, e.g., 20%, piperidine in DMF, and (b) reacting the product of step (a) with Fmoc-Lys(Palmitoyl-Glu-OtBu)-OH (IX):

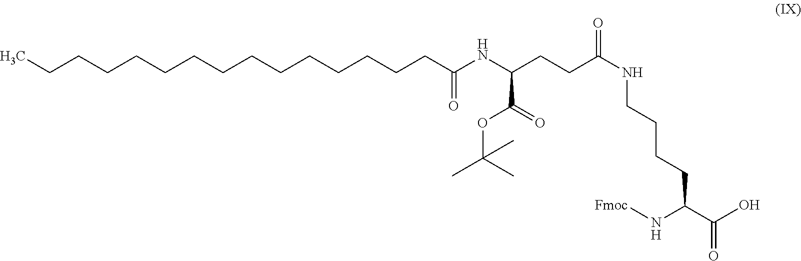

under amide bond-forming conditions, optionally wherein the amide bond-forming conditions comprise use of ethyl cyano (hydroxyimino) acetate (e.g., OxymaPure™) and N,N′-Diisopropylcarbodiimide (DIC).

In some embodiments, the method comprises the steps of (c) treating a resin-bound peptide of formula (X):

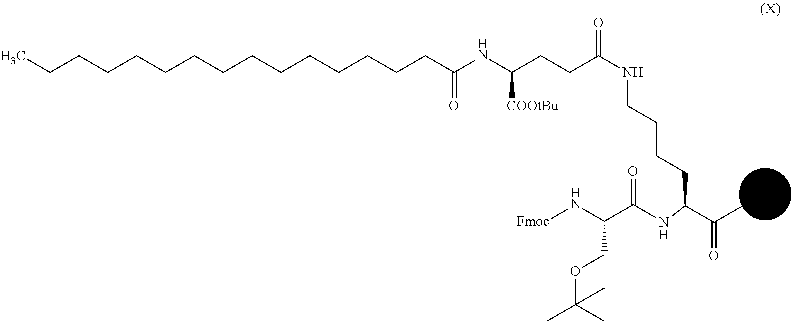

with 10 to 30%, e.g. 20%, piperidine in DMF, and (d) reacting the product of step (c) with Fmoc-Pro-Pro-OH (XI):

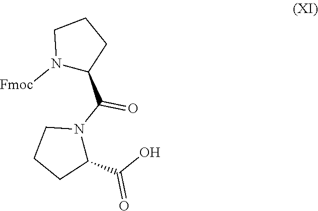

under amide bond-forming conditions, optionally wherein the amide bond-forming conditions comprise use of ethyl cyano (hydroxyimino) acetate (e.g., OxymaPure™) and DIC.

In some embodiments, the method comprises the steps of (e) treating a resin-bound peptide of formula (XII):

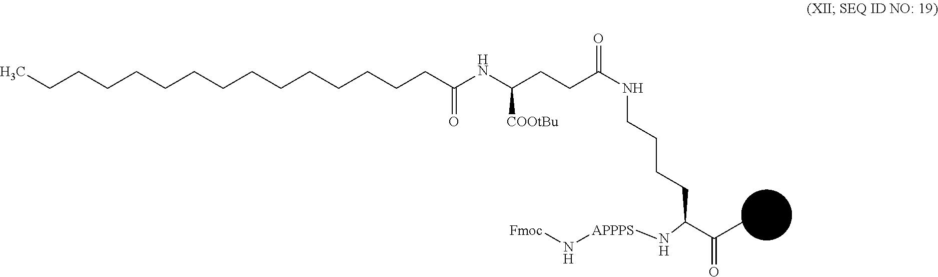

with 10 to 30%, e.g. 20%, piperidine in DMF, and (f) reacting the product of step (e) with Fmoc-Ser(tBu)-Gly-OH (XIII):

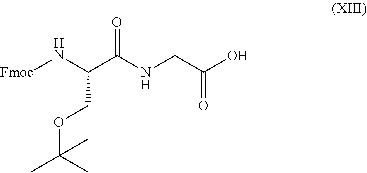

under amide bond-forming conditions, optionally wherein the amide bond-forming conditions comprise use of ethyl cyano (hydroxyimino) acetate (e.g., OxymaPure™) and DIC.

In some embodiments, the method comprises the steps of (g) treating a resin-bound peptide of formula (XIV):

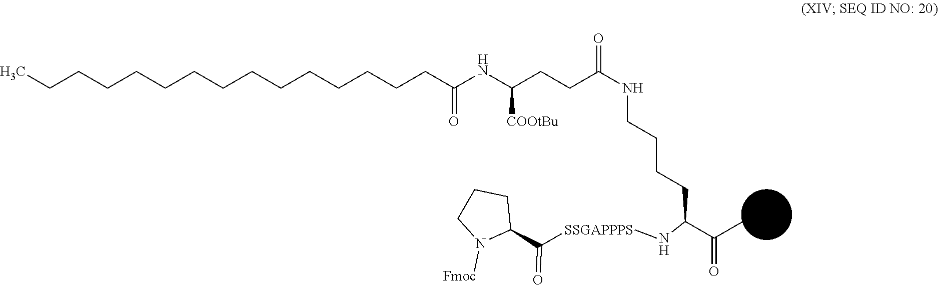

with 10 to 30%, e.g. 20%, piperidine in DMF, and (h) reacting the product of step (g) with Fmoc-Gly-Gly-OH (XV):

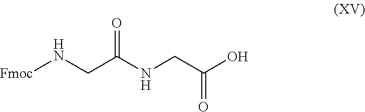

under amide bond-forming conditions, optionally wherein the amide bond-forming conditions comprise use of ethyl cyano (hydroxyimino) acetate (e.g., OxymaPure™) and N,N′-Diisopropylcarbodiimide.

In some embodiments, the method comprises the steps of (i) treating a resin-bound peptide of formula (XVI):

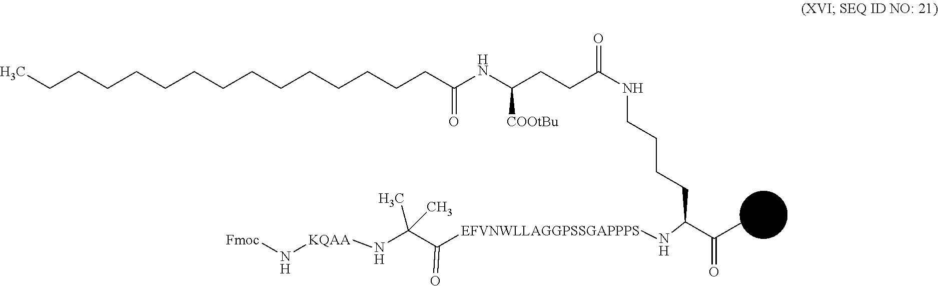

with 10 to 30%, e.g. 20%, piperidine in DMF, and (j) reacting the product of step (i) with Fmoc-Asp(OMpe)-OH (XVII):

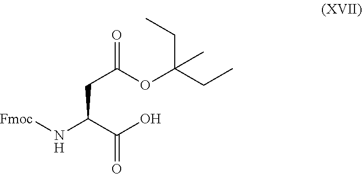

under amide bond-forming conditions, optionally wherein the amide bond-forming conditions comprise use of ethyl cyano (hydroxyimino) acetate (e.g., OxymaPure™) and (DIC).

In some embodiments, the method comprises the steps of (k) treating a resin-bound peptide of formula (XVIII):

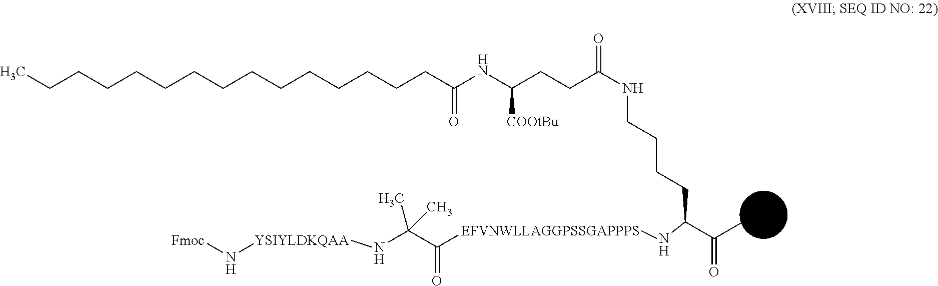

with 10 to 30%, e.g. 20%, piperidine in DMF, and (l) reacting the product of step (k) with Fmoc-Asp(OMpe)-OH (XVII):

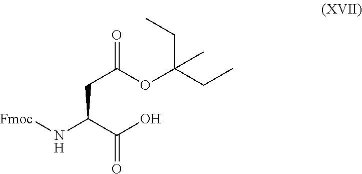

under amide bond-forming conditions, optionally wherein the amide bond-forming conditions comprise use of ethyl cyano (hydroxyimino) acetate (e.g., OxymaPure™) and DIC.

In some embodiments, the method comprises the steps of (m) treating a resin-bound peptide of formula (XIX):

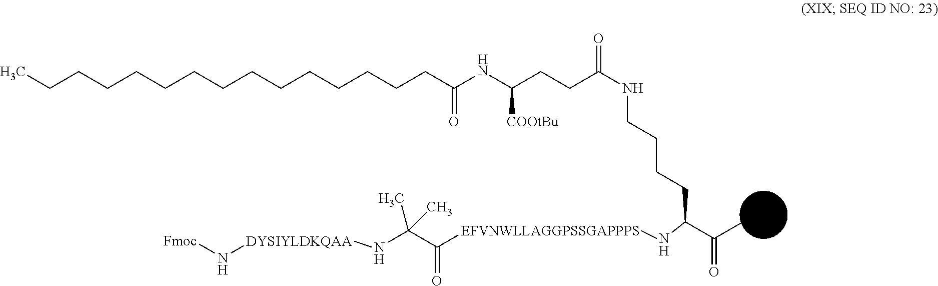

with 10 to 30%, e.g., 20%, piperidine in DMF, and (n) reacting the product of step (m) with Fmoc-Thr(tBu)-Ser(Y(Me,Me) pro)-OH (XX):

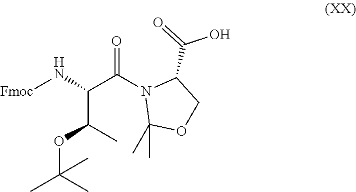

under amide bond-forming conditions, optionally wherein the amide bond-forming conditions comprise use of ethyl cyano (hydroxyimino) acetate (e.g., OxymaPure™) and DIC. The present methods may also comprise a monitoring step, optionally wherein the monitoring step comprises use of a colorimetric test selected from a Ninhydrin test, a Kaiser test, a Bromophenol blue test, a Chloranil test, or a 2,4,6-trinitrobenzenesulfonic acid (TNBS) test.

In some embodiments, the methods also comprise a purifying step.

In some embodiments, the purifying step comprises a chromatographical method, a method of tangential flow filtration, an ion exchange method, a lyophilization, or a combination thereof. In a particular embodiment, the purifying step comprises a preparative liquid chromatography, a tangential flow filtration, an ion exchange method, and a lyophilization.

In some embodiments, the purifying step comprises a preparative liquid chromatography step, optionally wherein the mobile phase used comprises ammonium acetate and acetonitrile.

In some embodiments, the purifying step comprises tangential flow filtration followed by ion exchange into a sodium salt by addition of 1M Na₂CO₃ solution, and lyophilization.

Also provided is a purifying step that comprises use of column chromatography and a triethylammonium phosphate (TEAP) mobile phase at pH 5.4 and/or use of column chromatography and a 0.1% TFA mobile phase.

Also provided is a composition comprising CT-868 in 0.1 M NH₄HCO₃ pH 10, optionally wherein CT-868 is at a concentration of 15 g/L or 20 g/L and a method of preparing this composition, comprising dissolving lyophilized CT-868 ammonium salt in 0.1 M NH₄HCO₃ pH 10. 28. This disclosure also provides an N-terminal conjugated peptidyl compound of Formula (I) or Formula (VI) made by any one of the methods herein, or a composition comprising an N-terminal conjugated peptidyl compound of Formula (I) or Formula (VI).

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is an overlay of the chromatograms observed for the CT-868 standard, pH 9, pH 10, lyophilizate 6-1, and lyophilizate 6-2 samples.

FIG. 2 is an overlay showing the alignment between the mass spectrometry (MS) spectra and UV chromatogram. Samples were aligned based on the main peak and/or impurities in order to ensure the correct time range for the MS data.

FIG. 3A shows the full scale mass spectra for the CT-868 Lyophilizate pH 9 sample. FIG. 3B shows the expanded scale mass spectra for freshly prepared CT-868_pH 9. FIG. 3C shows the MS spectrum of the main peak according to UV chromatogram of the CT-868 pH 9 sample.

FIG. 4 shows the MS spectrum of the main peak according to UV chromatogram of the CT-868 standard. The impurity (M-231) Mass 4410.2307 is incongruent.

FIG. 5 shows the MS spectrum of the main peak according to UV chromatogram of the CT-868_pH 10 sample. The impurity (M-231) Mass 4410.2307 is incongruent.

FIGS. 6A and 6B show, respectively, the chromatogram and MS spectrum of the main peak according to UV chromatogram of CT-868_6-1_Lyo sample.

FIGS. 7A and 7B show, respectively, the chromatogram and MS spectrum of the main peak according to UV chromatogram of CT-868_6-2_Lyo sample.

FIG. 8 shows an overlay chromatogram of a representative Stage 4 primary purification run (Run 4-1) analyzed by analytical UHPLC system 1 (C8) and system 2 (C18).

FIG. 9 shows the overlay of CT-868 in 10 mM NH₄HCO₃ at pH 8, pH 9, and pH 10 as analyzed by analytical UHPLC System 2 (C18).

FIG. 10 shows an overlay of Stage 4 primary purification run 4-1 (TEAP pH 5.4 mobile phase system) and run 4-2 (TEAP pH 6.5 mobile phase system) analyzed by analytical UHPLC System 2 (C18).

FIG. 11 shows analytical UHPLC analysis of Run 5-1 by System 2 (C18).

FIG. 12 is an overlay of stage 6 purification run 6-1 (0.1 M NH₄HCO₃ pH 8 mobile phase system) and run 6-2 (0.1 M NH₄HCO₃ pH 9 mobile phase system) analyzed by analytical UHPLC System 2 (C18).

FIG. 13 shows an overlay of analytical UHPLC analysis by System 2 (C18) of the dissolved CT-868 peptide in 0.1 M NH₄HCO₃ solution at pH 9 and pH 10.

FIG. 14 shows an overlay of analytical UHPLC analysis by system 2 (C18) of the in-process lyophilized CT-868 ammonium salt from Run 6-3 samples stored at room temperature over seven days.

FIG. 15 shows an overlay of analytical UHPLC analysis by System 2 (C18) of lyophilized CT-868 ammonium salt the run 6-3 samples dried at 35° C. over seven days.

FIG. 16 shows the chromatogram of the HPLC analysis of the isolated product of Example 3 after lyophilization.

### DETAILED DESCRIPTION OF THE INVENTION

The present disclosure provides a novel synthetic method for GLP-1R/GIPR agonistic peptide mimetic. This peptide mimetic is represented by formula (I):

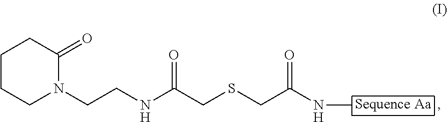 [SMILES: CNC(=O)CSCC(=O)NCCN1CCCCC1=O]

wherein Sequence Aa represents a peptidyl structure. The present synthetic method allows synthesis of the chemical moiety conjugated to Sequence Aa with improved handleability, purity, and yield.

The agonist herein agonizes the activity of GLP-1 and GIP. As used herein the term “native GLP-1” refers to a peptide comprising the sequence of human GLP-1 (7-36 or 7-37), and the term “native GIP” refers to a peptide comprising the sequence of human GIP (1-42). As used herein, a general reference to “GLP-1” or “GIP” in the absence of any further designation is intended to mean native GLP-1 or native GIP, respectively. In some embodiments, the agonist herein has at least 50% (e.g., at least 60, 70, 80, 90, 95, or 99%) of GLP-1R activation activity of native GLP-1-OH or GLP-1-NH₂ and/or at least 50% (e.g., at least 60, 70, 80, 90, 95, or 99%) of the GIPR activation activity of native GIP.

The present methods for synthesizing the peptide (I), GLP-1R/GIPR agonists (VI)-(XVI), and their intermediate (III) have several advantages over previously reported synthetic procedures, including enhanced yield and/or purity. The use of tricyclic amide resin may decrease the possibility of the chances of side reactions compared to the use of other solid phase peptide synthesis resins. The use of coupling reagents contemplated herein (e.g., TBTU or the combination of ethyl cyano (hydroxyimino) acetate (e.g., OxymaPure™) and N,N′-diisopropylcarbodiimide (DIC)) may decrease costs while also maintaining high coupling efficiency and yields. Furthermore, the reaction conditions for the addition of the 2-((2-(2-oxopiperidin-1-yl)ethylcarbamoyl)methylthio) acetic acid or the use of ethyl cyano (hydroxyimino) acetate (e.g., OxymaPure™) and N,N′-diisopropylcarbodiimide (DIC) avoid difficulties with solubility.

The choice of the amino acid building blocks described herein may also be advantageous. In particular, the use of Fmoc-Asp (Ompe) may decrease the risk of side reactions compared to use of other Fmoc-Asp SPPS building blocks. The use of dipeptides may decrease the incidence of deletion productions resulting from incomplete couplings. In addition, the use of a proline-proline dipeptide may minimize side reactions and diketopiperazine formation.

The use of a pseudoproline building block may also be advantageous. Pseudoprolines artificially created dipeptides that consist of serine- or threonine-derived oxazolidines and cysteine-derived thiazolidines. The name pseudoproline is based on the structural similarity with the cyclic amino acid proline. The presence of Y Pro within a peptide sequence results in the disruption of β-sheet peptide structures that are considered as a source of intermolecular aggregation. Thus, compared to the standard peptide, the pseudoproline dipeptides prevent or limit the formation of aggregates and thus allow the activated amino acid derivative to better assemble with the N-terminus of the growing peptide chain. Incorporating pseudoproline dipeptides into the peptide sequence enhances the coupling efficacy, and in turn, improves the purity and yield of the peptide. When treated with trifluoroacetic acid (TFA), the pseudoproline peptides are converted into serine, threonine or cysteine.

### I. Sequence Aa

In some embodiments, the peptidyl portion of the compound herein, Sequence Aa, comprises the formula W—R⁵, wherein W represents a peptidyl structure and R⁵ represents a moiety conjugated to the C-terminus of the peptidyl structure.

### A. Peptidyl Structure W

The peptidyl structure W may comprise an amino acid sequence that is present in native GLP-1, with either an —OH or —NH₂ group at the C terminus (i.e., GLP-1-OH or GLP-1-NH₂). The peptidyl structure may also comprise an amino acid sequence present in native GIP. For example, the peptidyl structure may comprise a hybrid sequence having one or more amino acid sequence fragments (e.g., functional fragments) present in native GLP-1 and one or more amino acid sequence fragments (e.g., functional fragments) present in native GIP.

In some embodiments, W has the following formula:

(SEQ ID NO: 1)
EGT(Xaa4)(Xaa5)SD(Xaa8)S(Xaa10)(Xaa11)(Xaa12)
(Xaa13)(Xaa14)(Xaa15)(Xaa16)(Xaa17)(Xaa18)(Xaa19)
(Xaa20)(Xaa21)(Xaa22)WL(Xaa25)(Xaa26)(Xaa27)GPSS
GAPPP(Xaa37),

wherein:
Xaa4 is F;
Xaa5 is T or I (e.g., T);
Xaa8 is Y, V, L, or K* (e.g., Y);
Xaa10 is I or S (e.g., I);
Xaa11 is Y, Y*, Q, A, or (Aib) (e.g., Y);
Xaa12 is L, M, or L* (e.g., L);
Xaa13 is D or E (e.g., D);
Xaa14 is K, G, R, or E (e.g., K);
Xaa15 is Q or I (e.g., Q);
Xaa16 is A, H, or R (e.g., A);
Xaa17 is A, Q, or V (e.g., A);
Xaa18 is A, (Aib), K*, K, or Q (e.g., (Aib));
Xaa19 is A, D, E, (Aib), or L (e.g., A, D, E, or L (e.g., E));
Xaa20 is For A (e.g., F);
Xaa21 is V or I (e.g., V);
Xaa22 is N, A, Q, K*, or E (e.g., N);
Xaa25 is I, L or V (e.g., L);
Xaa26 is A, K, or I (e.g., A);
Xaa27 is Q-R, G-R-G-K* (SEQ ID NO: 24), Q, or G (e.g., G); and
Xaa37 is S or absent (e.g., S).
   In such embodiments, the nitrogen atom directly connected to Sequence Aa in formula (I) is the amino group of the N-terminal glutamate (E) amino acid of W.

In some embodiments, W has the following formula:

(SEQ ID NO: 3)
EGTF(Xaa5)SD(Xaa8)S(Xaa10)(Xaa11)(Xaa12)(Xaa13)
(Xaa14)QA(Xaa17)(Xaa18)(Xaa19)F(Xaa21)(Xaa22)WL
(Xaa25)(Xaa26)GGPSSGAPPPS,

wherein:
Xaa5 is T or I (e.g., T);
Xaa8 is Y, V, or L (e.g., Y);
Xaa10 is I or S (e.g., I);
Xaa11 is Y, Q, or A (e.g., Y);
Xaa12 is L, M, or L* (e.g., L);
Xaa13 is D or E (e.g., D);
Xaa14 is K, G, or E (e.g., K);
Xaa17 is A or V (e.g., A);
Xaa18 is (Aib) or K (e.g., (Aib));
Xaa19 is E or L (e.g., E);
Xaa21 is V or I (e.g., V);
Xaa22 is N, A, or E (e.g., N);
Xaa25 is L or V (e.g., L); and
Xaa26 is A or K (e.g., A).
   In such embodiments, the nitrogen atom directly connected to Sequence Aa in formula (I) is the amino group of the N-terminal glutamate (E) amino acid of W.

In some embodiments, W has the following formula:

(SEQ ID NO: 4)
EGTF(Xaa5)SD(Xaa8)S(Xaa10)(Xaa11)(Xaa12)(Xaa13)
(Xaa14)QA(Xaa17)(Aib)(Xaa19)F(Xaa21)(Xaa22)WL
(Xaa25)(Xaa26)GGPSSGAPPPS,

wherein each of the “Xaa” variables is as defined above. In such embodiments, the nitrogen atom directly connected to Sequence Aa in formula (I) is the amino group of the N-terminal glutamate (E) amino acid of W.

In some embodiments, W has the following formula:

(SEQ ID NO: 2)
EGTFTSDYSIYLDKQAA(Aib)EFVNWLLAGGPSSGAPPPS.

As used herein, “(Aib)” refers to 2-aminoisobutyric acid (also known as α-aminoisobutyric acid or a-methylalanine or 2-methylalanine).

As used herein, Y* refers to 2-amino-3-(4-hydroxyphenyl)-2-methylpropanoic acid (e.g., (S)-2-amino-3-(4-hydroxyphenyl)-2-methylpropanoic acid).

As used herein, L* refers to 2-amino-2-methylpentanoic acid (e.g., (S)-2-amino-2-methylpentanoic acid) or a C-terminal amino acid or an amino acid ester or an amino acid amide thereof.

As used herein, K* is a lysine residue substituted with a modifying group, or a C-terminal amino acid or an amino acid ester or amino acid amide thereof.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics or substitutions of residues with similar side chain volume are also within the scope of this disclosure.

Amino acids can be grouped according to similarities in the properties of their side chains (see, e.g., A L. Lehninger, in Biochemistry, 2^{nd} Ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H).

Alternatively, naturally occurring residues can be divided into groups based on common side-chain properties: (1) hydrophobic: norleucine, Met, Ala, Val, Leu, Ile, Phe, Trp; (2) neutral hydrophilic: Cys, Ser, Thr, Asn, Gln, Ala, Tyr, His, Pro, Gly; (3) acidic: Asp, Glu; (4) basic: His, Lys, Arg; (5) residues that influence chain orientation: Gly, Pro; (6) aromatic: Trp, Tyr, Phe, Pro, His, or hydroxyproline. Non-conservative substitutions will entail exchanging a member of one of these classes for another class.

In some embodiments, conservative substitutions for use in the variants described herein are as follows: Ala into Gly or into Ser; Arg into Lys; Asn into Gln or into His; Asp into Glu or into Asn; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr or into Phe; Tyr into Phe or into Trp; and/or Phe into Val, into Tyr, into Ile or into Leu.

In general, conservative substitutions encompass residue exchanges with those of similar physicochemical properties (e.g., substitution of a hydrophobic amino acid residue for another hydrophobic amino acid residue).

In some embodiments, W includes one or more naturally occurring amino acids found, e.g., in polypeptides and/or proteins produced by living organisms, such as Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H).

In some embodiments, W includes one or more independently selected modifications that occur in so-called modified peptides. Such modifications include, but are not limited to: (i) the incorporation of lactam-bridge; (ii) head-to-tail cyclization; (iii) one or more alternative or non-naturally occurring (D- or L-) amino acids, such as synthetic non-native amino acids, substituted amino acids, and D-amino acids; (iv) peptide bond replacements; (v) targeting groups; and the like. In some embodiments, the peptide includes one modification in either W or R⁵. In other embodiments, the peptide includes more than one independently selected modification (e.g., 2 independently selected modifications, 3 independently selected modifications, 4 independently selected modifications, 5 independently selected modifications, 6 independently selected modifications, 7 independently selected modifications, 8 independently selected modifications, 9 independently selected modifications, or 10 independently selected modifications) that occur in W and/or R⁵ (e.g., in W only; or in R⁵ only; or in both W and R⁵).

Non-limiting examples of alternative or non-naturally occurring amino acids include D-amino acids, beta-amino acids, homocysteine, phosphoserine, phosphothreonine, phosphotyrosine, hydroxyproline, gamma-carboxyglutamate, hippuric acid, octahydroindole-2-carboxylic acid, statine, 1,2,3,4-tetrahydroisoquinoline-3-carboxylic acid, penicillamine (3-mercapto-D-valine), ornithine, citruline, alpha-methyl-alanine, para-benzoylphenylalanine, para-ammo phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-Icyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof (each which can independently be D- or L-amino acids).

The peptidyl structure W may comprise one or more non-natural peptide bonds. Non-limiting examples of peptide bond replacements include urea, thiourea, carbamate, sulfonyl urea, trifluoroethylamine, ortho-(aminoalkyl)-phenylacetic acid, paras (aminoalkyl)-phenylacetic acid, meta-(aminoalkyl)-phenylacetic acid, thioamide, tetrazole, boronic ester, olefinic group, and derivatives thereof. Unless otherwise indicated, the peptide bonds herein are naturally occurring peptide bonds.

In some embodiments, W includes only naturally occurring amino acids. In other embodiments, W includes only alternative or non-naturally occurring amino acids. In still other embodiments, W includes one or more naturally occurring amino acids and one or more alternative or non-naturally occurring amino acids. In some of the foregoing embodiments, W includes only L-amino acids; or W includes both D- and L-amino acids; or W includes only D-amino acids. While not wishing to be bound by theory, it is believed that the incorporation of D-amino acids can confer enhanced in vivo or intracellular stability to the compounds described herein.

### B. R⁵ Moiety

In some embodiments, R⁵ is a C-terminal amino acid amide that is optionally substituted with 1 or 2 modifying groups (e.g., 1 or 2 groups selected from an acyl group and a PEG group). In other embodiments, R⁵ is a C-terminal amino acid that is optionally substituted with 1 or 2 modifying groups (e.g., 1 or 2 groups selected from an acyl group and a PEG group).

In some embodiments, R⁵ is a C-terminal lysyl amide residue that is optionally substituted with 1 or 2 modifying groups (e.g., 1 or 2 groups selected from an acyl group and a PEG group). In some embodiments, R⁵ is a C-terminal L-lysyl amide residue that is optionally substituted with 1 or 2 modifying groups (e.g., 1 or 2 groups selected from an acyl group and a PEG group).

In some embodiments, R⁵ has the formula (IV) or (XXI):

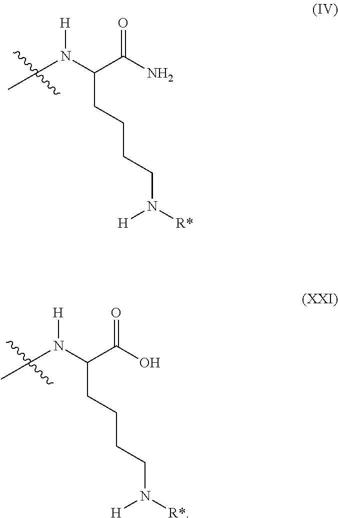

wherein R* is H or a modifying group (e.g., an acyl group or a PEG group). In some embodiments, formula (II) or (III) represents an L-amino acid. In other embodiments, formula (II) or (III) represents a D-amino acid.

In some embodiments, R* is H.

In some embodiments, the modifying group (i.e., R*) is an acyl group. In further embodiments, R* is a C₂₋₃₀ (e.g., C₂₋₂₀, C₂₋₁₀, C₂₋₆) acyl group that is optionally substituted with 1 or 2 independently selected R^{f}. Each occurrence of R^{f} may be selected from the group consisting of —C(═O)(OH); —C(═O)(C₂₋₂₀ alkyl); —C(═O)O(C₂₋₂₀ alkyl); —P(═O)(OH)₂; and —S(O)₁₋₂(C₁₋₆ alkyl); oxo; F; C₁₋₁₀ alkoxy; C₁₋₁₀ haloalkoxy; and —N(R^{g})(R^{h}). In some embodiments, each occurrence of R^{f} is independently selected from the group consisting of-C(═O)(OH) and —N(R^{g})(R^{h}). Each occurrence of Re and Rh is independently selected from the group consisting of H; C₁₋₄ alkyl; —C(═O)(C₂₋₂₀ alkyl); —C(═O)O(C₂₋₂₀ alkyl); and —S(O)₁₋₂(C₁₋₆ alkyl).

In some embodiments, the modifying group (i.e., R*) has the formula (V):

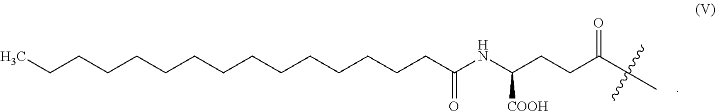 [SMILES: *C(=O)CC[C@H](NC(=O)CCCCCCCCCCCCCCC)C(=O)O]

### C. Exemplary Sequence Aa

In some embodiments, the W—R⁵ structure herein has the following formula (XXIII):

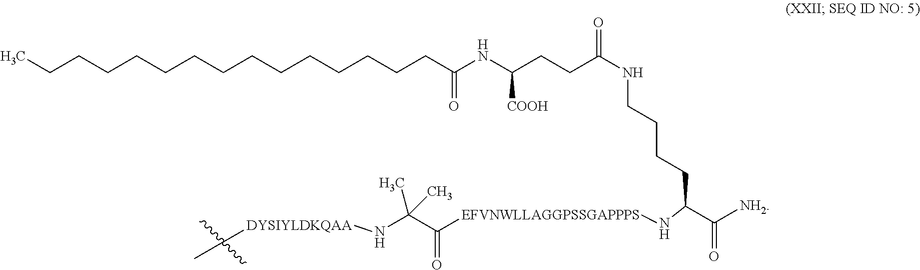

In such embodiments, the nitrogen atom directly connected to Sequence Aa in formula (I) is the amino group of the N-terminal glutamate (E) amino acid of W.

In some embodiments, the W—R⁵ structure may have any one of the following formulae (XXIII-XXXI):

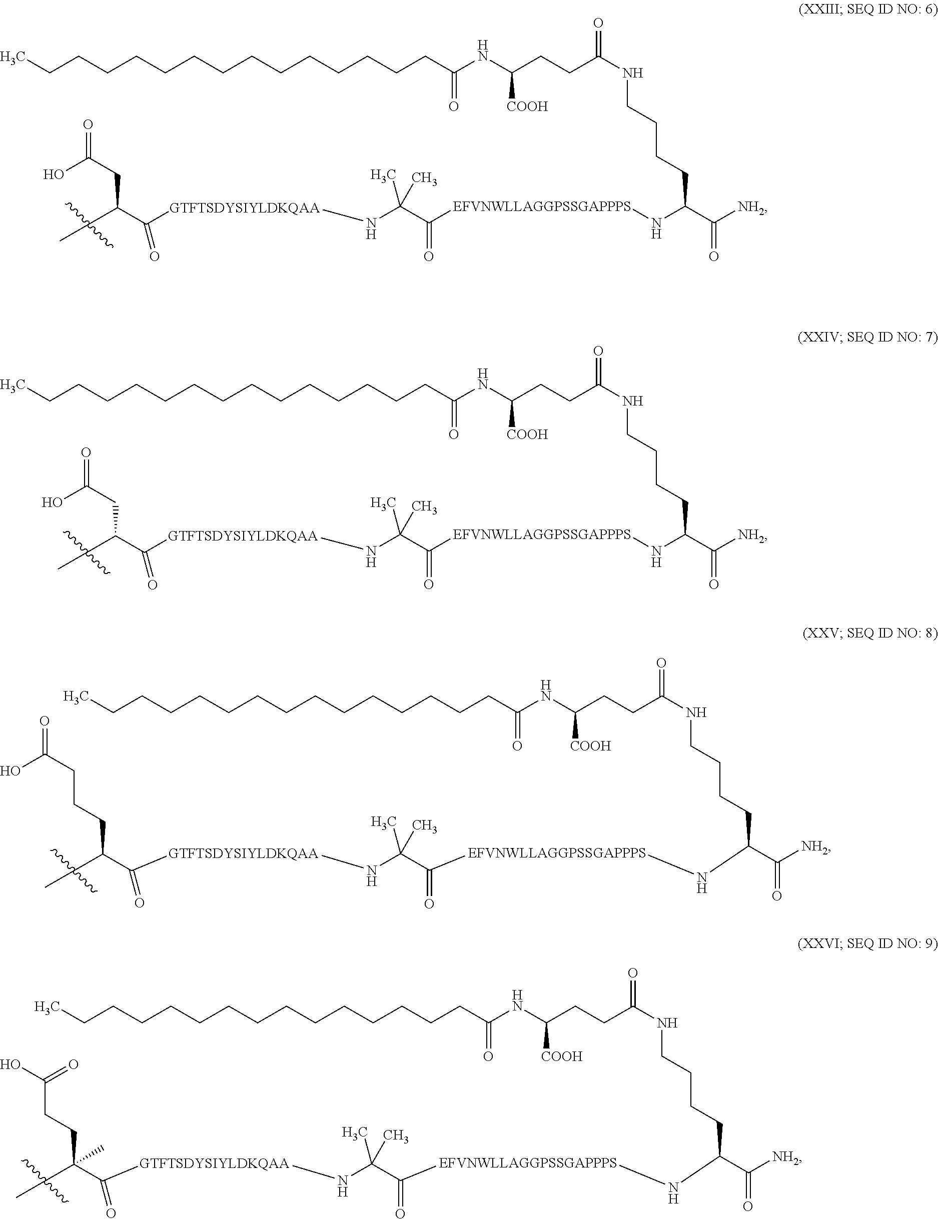

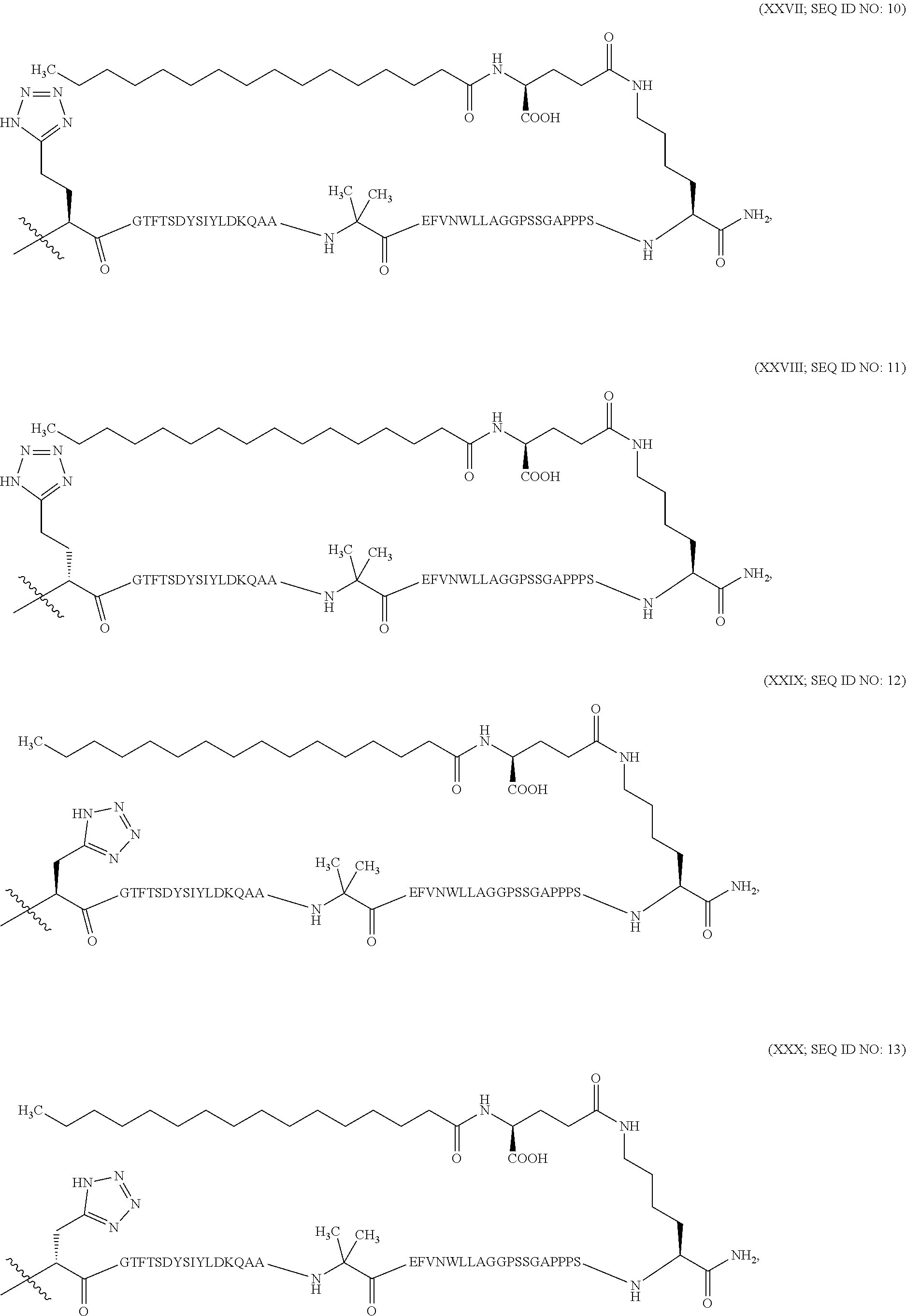

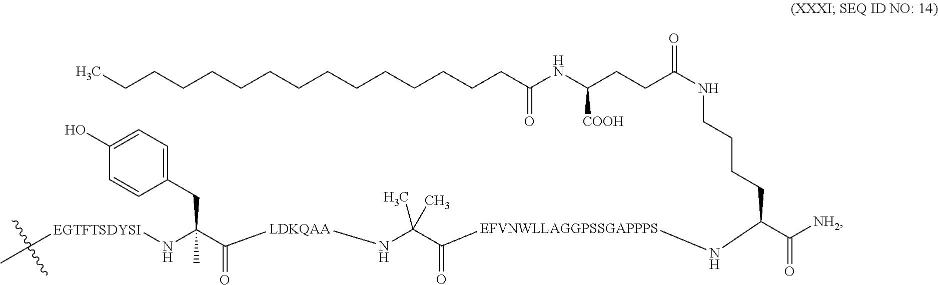

wherein the nitrogen atom directly connected to Sequence Aa in formula (I) is the amino group of the N-terminal glutamate (E) amino acid of W, and

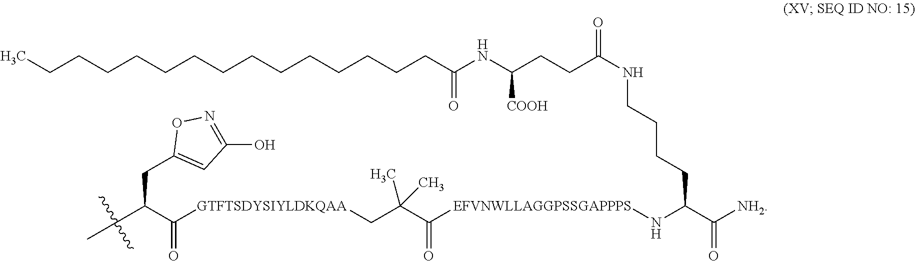

As used in SEQ ID NOs 6-13 and 15, [symbol] indicates the point of attachment of the peptide to the chemical structure in Formula (I).

To illustrate the conjugation between carboxylic acid (III) and W—R⁵, a nonlimiting exemplary dual GLP-1R/GIPR agonist has the following structural formula (VI):

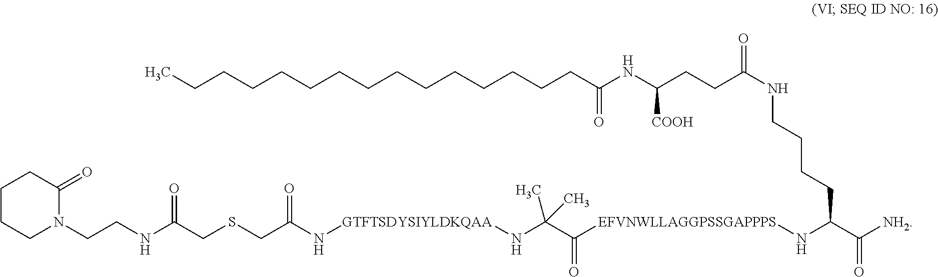

The dual GLP-1R/GIPR agonist of formula (VI) is a particular embodiment of an N-terminal conjugated peptidyl compound of formula (I) and is hereinafter designated as CT-868.

Unless otherwise indicated, the one-letter abbreviations used to describe amino acid residues in SEQ ID NOs: 4-15 are amino acid residues in their native configurations connected by native peptidyl bonds.

### II. Synthesis of Peptidyl Moiety

In some embodiments, the present method comprises the steps of using standard solid-phase synthetic procedures to produce a resin-bound peptide having the structure (VII):

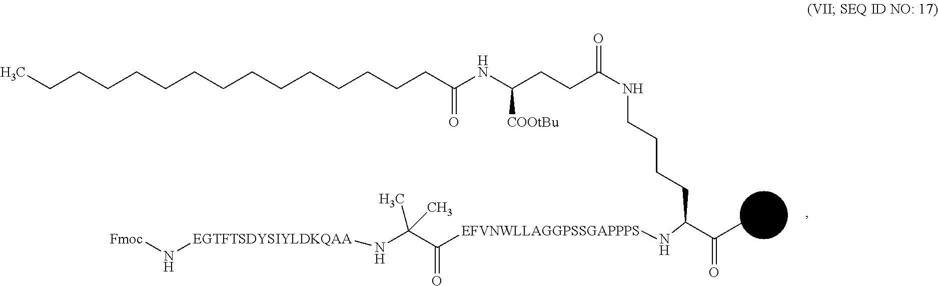

referred to in some embodiments as (II):

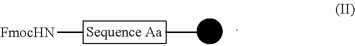

Throughout this application (e.g., in formulas (II) and (VII) above) the resin is represented using a solid black circle:

[symbol]

A resin-bound peptide comprises all non-Fmoc labile protecting groups that were used in the synthesis of that peptide. In particular, an “S” residue in the resin-bound peptide depicted above should be understood as still comprising a hydroxy protecting group (e.g., tBu or Y (Me,Me) pro); a “W” residue in the resin-bound peptide depicted above should be understood as still comprising a nitrogen protecting group (e.g., Boc); an “N” residue in the resin-bound peptide depicted above should be understood as still comprising a nitrogen protecting group (e.g., Trt); an “E” residue in the resin-bound peptide depicted above should be understood as still comprising a carboxylic acid protecting group (e.g., OtBu); a “Q” residue in the resin-bound peptide depicted above should be understood as comprising a nitrogen protecting group e.g., Trt); a “K” residue in the resin-bound peptide depicted above should be understood as still comprising an amine protecting group (e.g., Boc); a “Y” residue in the resin-bound peptide depicted above should be understood as still comprising a hydroxy protecting group (e.g., tBu); a “D” residue in the resin-bound peptide depicted above should be understood as still comprising a carboxylic acid protecting group (e.g., OMpe); and a “T” residue in the resin-bound peptide depicted above should be understood as till comprising a hydroxy protecting group (e.g., tBu). Such protecting groups are acid labile and are removed once the peptide is cleaned from the resin.

Solid-phase synthetic methods are well known in the art, and include an iterative cycle of fluorenylmethyloxycarbonyl (Fmoc) deprotection, amino acid coupling, washes, and optionally capping (see, e.g., W. C. Chan and P. D. White, Fmoc Solid Phase Peptide Synthesis: A Practical Approach, Oxford University Press Inc., 1999). The solid-phase peptide synthesis may be carried out using a solid support (e.g., a resin). The solid support typically consists of a polymeric resin, most commonly low cross-linked polystyrene beads, which is functionalized with reactive groups to enable covalent binding between the carboxyl group of the first amino acid of the nascent peptide chain and the resin through a linker. The most common polymeric solid support used is a resin composed of a 1-2% divinylbenzene-cross-linked polystyrene. Furthermore, a resin is composed of the polymeric solid support linked permanently to a linker (bifunctional spacer, or handle) that facilitates temporary anchoring of the first amino acid to the polymeric solid support. Depending on the type of linker, the C-terminus of the first amino acid is anchored to the solid support as an amide, ester, thioester, O-substituted oxime, or hydrazide. For instance, the Rink amide resin comprises benzhydrylamine and the Sieber amide resin comprises xanthenylamine.

The resin may be, for example, Sieber amide resin, Ramage amide resin (also known as tricyclic amide resin), or Rink amide methylbenzhydryl amine (MBHA) resin. After cleavage from each of these resins, the peptide comprises a C-terminal amide group.

In particular embodiments, the resin is a Ramage amide AM resin.

### Washing and Fmoc Deprotection

Resin-bound Fmoc-protected amines may be deprotected through use of 20% piperidine in DMF for 15-35 minutes.

### Amino Acid Coupling

The solid-phase synthetic methods may comprise use of N,N′-Diisopropylcarbodiimide (DIC) and Ethyl cyano (hydroxyimino) acetate (e.g., OxymaPure™) in a polar aprotic solvent (e.g., DMF). In some embodiments, the Fmoc-protected amino acid unit is preactivated with DIC and ethyl cyano (hydroxyimino) acetate (e.g., OxymaPure™) in a polar non-protic solvent (e.g., DMF) in, a non-reactive vessel (e.g., a high-density polyethylene (HDPE) bucket). In some embodiments, the preactivation comprises stirring for 10 to 30, e.g. 20, minutes.

Each preactivation may comprise the use of 1.5 molar equivalents ethyl cyano (hydroxyimino) acetate (Oxymapure™) for each amino acid reagent and 1.3 molar equivalents DIC for each amino acid reagent. In a particular embodiment, 1.4 to 1.7 molar equivalents of ethyl cyano (hydroxyimino) acetate (OxymaPure™) for each equivalent amino acid reagent and 1.3 to 1.6 molar equivalents DIC for each equivalent amino acid reagent are used as coupling agent combination.

Alternatively, amino acid units may also be activated by use of DIC with 1-hydroxy-benzotriazole (HOBt) or 1-hydroxy-7-aza-benzotriazole (HOAt).

In some embodiments, once the optionally preactivated amino acid is added to the resin, the mixture is stirred for 10 to 30, e.g. 20, minutes. A second portion of DIC (e.g., 0.7 molar eq of DIC for the coupled amino acid unit) may be added after 20 minutes of stirring. Each coupling step may be carried out for 1 to 24 hours.

After addition of the optionally preactivated amino acid (e.g., one hour after addition of the optionally preactivated amino acid), the reaction may be monitored using a colorimetric test (e.g., the ninhydrin test, the Kaiser test, the Bromophenol blue test, the Chloranil test or the TNBS test (2,4,6-trinitrobenzenesulfonic acid test). In some embodiments, the reaction is not deemed complete until the colorimetric test(s) are negative.

In some embodiments, the same coupling step may be carried out two times, which can also be referred to herein as recoupling. In some embodiments, recoupling is used only for certain steps (e.g., for the first and 19th couplings described in Example 1, Table 3). A reduced amount (e.g., half) of the amino acid reagent, DIC, and ethyl cyano (hydroxyimino) acetate (e.g., OxymaPure™) may be used for the recoupling. Reaction completion may be monitored using colorimetric tests as discussed above.

In some embodiments couplings may be performed as follows:
1. Dissolve Fmoc-protected amino acid and 55.4 g ethyl cyano (hydroxyimino) acetate (OxymaPure™) in DMF in a high density poly ethylene (HDPE) bucket equipped with a magnetic stir bar and placed on a magnetic stir plate.
2. Record the temperature of the amino acid solution before DIC addition, after DIC addition, and after pre-activation is complete. Add 53 ml of DIC (1st Addition) using an addition funnel to the amino acid solution. After addition, stir for 20-30 minutes to pre-activate.
3. Add the activated amino acid solution to the reactor. If necessary, add enough additional DMF to make the resin bed fluid. 20 minutes after adding the amino acid solution to the reactor, add 29 ml of DIC (2nd Addition). The coupling time for this reaction is 1-24 hours.
4. After 1 hour of coupling, monitor the reaction periodically using the Ninhydrin and TNBS colorimetric tests until negative results are achieved or until the 24-hour maximum coupling time has been reached. If the results are negative, stop the reaction, record the coupling stop time, and then proceed with Recoupling (where appropriate). After 24 hours, drain the reactor, record the coupling stop time, and perform a final set of colorimetric tests.
5. Fill the reactor to a level 1.5 to 2.0 times the resin bed height with DMF, stir for a minimum of 3 minutes, and then drain the reactor.

### Re-Coupling: Steps 6-9 are Performed for Cycles 1 and 19 Only

6. Dissolve Fmoc-protected amino acid and 27.7 OxymaPure™ in DMF in an HDPE bucket equipped with a magnetic stir bar and placed on a magnetic stir plate.
7. Record the temperature of the amino acid solution before DIC addition, after DIC addition, and after pre-activation is complete. Add 27 ml of DIC (1st Addition) using an addition funnel to the amino acid solution. After addition, stir for 20-30 minutes to pre-activate.
8. Add the activated amino acid solution to the reactor. If necessary, add enough additional DMF to make the resin bed fluid. 20 minutes after adding the amino acid solution to the reactor, add 14 ml of DIC (2nd Addition). The coupling time for this reaction is 1-24 hours.
9. After 1 hour of coupling, monitor the reaction periodically using the Ninhydrin and TNBS colorimetric tests until negative results are achieved or until the 24-hour maximum coupling time has been reached. If the results are negative, stop the reaction, record the coupling stop time, and proceed with Capping. After 24 hours, drain the reactor, record the coupling stop time, and perform a final set of colorimetric tests. If the results are strongly positive, proceed with Recoupling (request an additional set of pages from QA). If the results are slightly positive or negative, proceed with Capping.

In some embodiments, the present methods comprise use of dipeptide building blocks. In some embodiments, the dipeptide building block is an Fmoc-Pro-Pro-OH building block. This building block may be used to install, e.g., the prolines at positions 35 and 36 in the above structure in a single coupling step (see, e.g., Cycle 3 of Example 1 and Example 1, Table 3).

In some embodiments, the dipeptide building block is an Fmoc-Ala-Pro-OH building block. This building block may be used to install, e.g., the alanine at position 33 and the proline at position 34 in the above structure in a single coupling step (see, e.g., Cycle 4 of Example 3, Table 18).

In some embodiments, the dipeptide building block is an Fmoc-Gly-Pro-OH building block. This building block may be used to install, e.g., the glycine at position 28 and the proline at position 29 in the above structure in a single coupling step (see, e.g., Cycle 8 of Example 3, Table 18).

In some embodiments, the dipeptide building block is an Fmoc-Ala-Gly-OH building block. This building block may be used to install, e.g., the alanine at position 26 and the glycine at position 27 in the above structure in a single coupling step (see, e.g., Cycle 9 of Example 3, Table 18).

In some embodiments, the present methods may comprise use of an Fmoc-Ser(tBu)-Gly-OH building block. This building block may be used to install, e.g., the glycine at position 32 and the serine at position 31 in the above structure in a single coupling step (see, e.g., Cycle 6 of Example 1 and Table 3).

In some embodiments, the present methods may comprise the use of an Fmoc-Gly-Gly-OH building block. This building block may be used to install, e.g., the glycines at positions 27 and 28 in the above structure in a single coupling step (see, e.g., Cycle 9 of Example 1 and Table 3).

In some embodiments, the present methods comprise us of an Fmoc-Asp(OMpe)-OH building block. This building block may be used to install, e.g., the aspartates at positions 7 and 13 in the above structure (see, e.g., Cycles 23 and 29 of Example 1 and Table 3).

In some embodiments, the present methods comprise use of pseudoproline building blocks. In some embodiments, the present methods comprise use of an Fmoc-Thr(tBu)-Ser (W (Me,Me) pro)-OH building block. This building block may be used to install, e.g., the threonine at position 5 and the serine at position 6 in the above structure in a single coupling step (see, e.g., Cycle 30 of Example 1 and Table 3).

In some embodiments, the present methods comprise use of an Fmoc-Gly-Thr (W (Me,Me) pro)-OH building block. This building block may be used to install, e.g., the threonine at position 3 and the glycine at position 2 in the above structure in a single coupling step (see, e.g., Cycle 31 of Example 3 and Table 18.

In some embodiments, the method comprises use of Fmoc-Lys (palmitoyl-y-Glu-OtBu)-OH. This building block may be used to install, e.g., the modified lysine at position 38 in the above structure in a single coupling step (see, e.g., Cycle 1 of Example 1 and Table 3, Cycle 1 of Example 3 and Table 18).

### Washes

In some embodiments, washes are carried out after each coupling step. These washes may comprise use of DMF or IPA. Each wash may be carried out for a suitable time, e.g. for a few (e.g., three) minutes).

### Capping

In some embodiments, capping of unreacted amines with acetic anhydride in the presence of a non-nucleophilic base (e.g., DIPEA or pyridine), optionally in an organic solvent such as DMF, is carried out. The capping step may be carried out following one or more coupling step(s). In some embodiments, the capping is carried out using a capping solution of acetic acid anhydride and pyridine in DMF (ratio: Jan. 1, 1950, v/v/v, 10 mL/g initial resin).

In some embodiments, capping is carried out as follows:

### Capping: Steps 10-18 are Performed for Cycles 1 and 19 Only

1. Wash the resin twice. For each wash, fill the reactor to a level 1.5 to 2.0 times the resin bed height with DMF, stir for a minimum of 3 minutes, and then drain the reactor.
2. Prepare the 20% DIPEA in DMF solution by adding 23 ml of DIPEA and 92 ml of DMF to a glass beaker or flask. Stir the mixture for a minimum of 1-3 minutes.
3. Prepare the 20% Ac₂O in DMF solution by adding 123 ml of Ac₂O and 492 ml of DMF to a glass beaker or flask. Stir the mixture for a minimum of 1-3 minutes.
4. Add enough DMF to the reactor to make the resin bed fluid, and begin stirring the resin. Add the prepared 20% DIPEA in DMF solution to the reactor.
5. Add the prepared 20% Ac₂O in DMF solution to the reactor
6. Stir for 15-30 minutes (time starts when addition of the 20% Ac₂O in DMF solution is complete), and then drain the reactor.
7. Wash the resin 5 times. For each wash, fill the reactor to a level 1.5 to 2.0 times the resin bed height with the listed solvent, stir for a minimum of 3 minutes, and then drain the reactor. Washes 1 and 3-5: DMF; wash 2: IPA.
8. Perform Ninhydrin and TNBS colorimetric testing. The results must be negative.
9. Perform washes for a minimum of three minutes each. Washes 1 and 3: DMF; wash 2: IPA.

### Process Monitoring

In-process monitoring may be carried out using methods known in the art. In particular, Fmoc deprotection may be monitored by the ninhydrin test. Amino acid coupling may be monitored by the TNBS colorimetric test. For cycles following the addition of Fmoc-Pro-OH, the chloranil colorimetric test may be used.

### III. Coupling of N-Terminal Carboxylic Acid and Cleavage from Resin

The dual GLP-1R/GIPR agonist herein may be synthesized by conjugating the N-terminus of Sequence Aa to a compound of formula (III) via an amide bond:

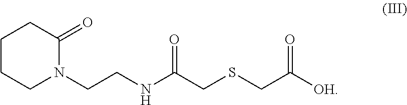 [SMILES: O=C(O)CSCC(=O)NCCN1CCCCC1=O]

This compound may be referred to as 2-((2-(2-oxopiperidin-1-yl)ethylcarbamoyl)methylthio) acetic acid (formula III).

Cycle 35 described in Example 1, which immediately follows cycle 34 in Example 1, Table 3, or Cycle 33 of Example 3, may be carried out in accordance with the embodiments below.

In some embodiments, the present method comprises one or more steps shown in the following synthetic scheme:

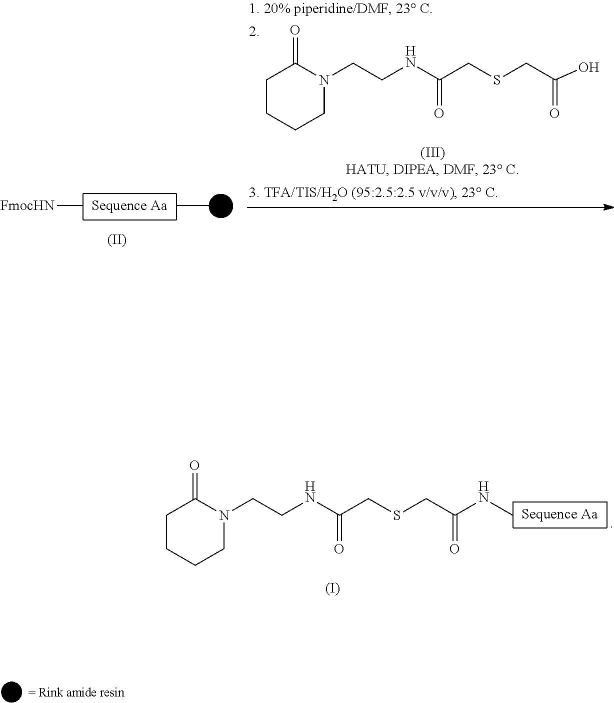

In some embodiments, the present method comprises the steps of using standard solid-phase synthetic procedures to produce a resin-bound peptide having the structure (VII):

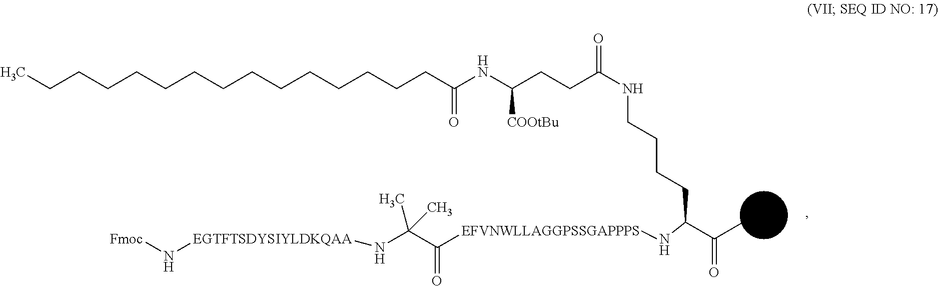

referred to in some embodiments as (II):

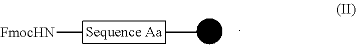

In some embodiments, the present method comprises the steps of deprotecting the resin-bound peptide of formula (XXII) or (XXIII) using 10 to 30%, e.g. 20%, piperidine in DMF. In some embodiments, the piperidine-deprotected resin-bound peptide is reacted with intermediate (III) under amide bond-forming coupling conditions. The amide bond-forming conditions may involve treating the intermediate (III) with a coupling reagent (e.g., hexafluorophosphate azabenzotriazole tetramethyl uronium (HATU) or 2-(1H-Benzotriazole-1-yl)-1,1,3,3-tetramethylaminium tetrafluoroborate (TBTU)) optionally in the presence of a weak base (e.g., diisopropyl ethyl amine) in a polar aprotic solvent (e.g., dimethyl formamide). In some embodiments, the reaction is carried out for 1 to 3 hours. In some embodiments, 2-(2-piperidon-1-yl)-ethylcarbamoylmethylthioacetic acid and TBTU are used, and the molar ratio of 2-(2-piperidon-1-yl)-ethylcarbamoylmethylthioacetic acid to TBTU is 1:0.82. In some embodiments, 2-(2-piperidon-1-yl)-ethylcarbamoylmethylthioacetic acid and TBTU are used in a ratio of 246.9 g 2-(2-piperidon-1-yl)-ethylcarbamoylmethylthioacetic acid to 253.0 g TBTU. In some embodiments, 2-(2-piperidon-1-yl)-ethylcarbamoylmethylthioacetic acid and TBTU are used in a ratio of 76.0 g 2-(2-piperidon-1-yl)-ethylcarbamoylmethylthioacetic acid to 73.1 g TBTU.

In a particular embodiment 2-(2-piperidon-1-yl)-ethylcarbamoylmethylthioacetic acid is coupled to a resin-bound peptide of formula (XXXIII):

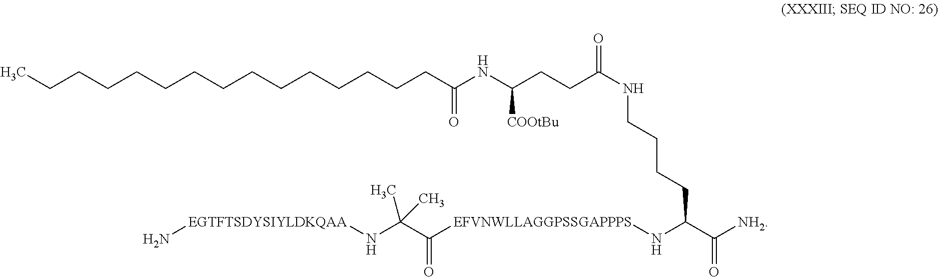

under amide bond-forming conditions comprising the combination of ethyl cyano (hydroxyimino) acetate (e.g., OxymaPure™) and N,N′-diisopropylcarbodiimide (DIC). In particular, a ratio of 1.4 to 1.7 equivalents ethyl cyano (hydroxyimino) acetate per one equivalent of peptide building block and 1.8 to 2.2 equivalents of DIC per one equivalent of peptide building block are used.

In some embodiments, after the intermediate (III) has been coupled to the resin-bound deprotected peptide via an amide bond, the conjugated peptide is cleaved from the resin using a strong acid (e.g., trifluoroacetic acid (TFA)) in the presence of a reducing agent or scavenger (e.g., triisopropyl silane or water). A suitable cleavage cocktail may comprise TFA, tri-isopropyl-silane (TIS), and H₂O (e.g., 90:5:5 v/v/volumes TFA/TIS/H₂O). Alternatively, cleavage mixtures may further comprise thiol-based scavengers, such as ethan-1,2-dithiol (EDT), 1,4-dithiothreitol (DTT), 1,4-dithioerythritol (DTE) or 2,2′-(ethylenedioxy) diethanethiol (DODT. An exemplary suitable cleavage cocktail with a thiol-based scavenger comprises TFA, TIS, EDT and water, for example TFA, EDT, TIS, and H₂O in a ratio of 90:5:2.5:2.5 v/v/v/v. In some embodiments, ammonium iodide and ascorbic acid are added following cleavage as reducing agents. In some embodiments, the scavenger is triisopropyl silane or water.

The crude product is isolated from the mixture by precipitation. In some embodiments, the precipitation is carried out with isopropyl ether (IPE).

The carboxylic acid of formula (III) (2-((2-(2-oxopiperidin-1-yl)ethylcarbamoyl)methylthio) acetic acid) may be produced from commercially available starting materials in greater than 5% (e.g., greater than 5%, 6%, 7%, 8%, 9%, 10% 11%, 12%, 13%, 14%, or 15%) yield.

### IV. Purification and Salt Exchange

### Analytical Methods

In some embodiments, an analytical method is used to assess the purity of the peptide. Such methods may use a hydrophobic column, such as a C8 or C18 column. In some embodiments, the column is a C18 column, such as a Water ACQUITY Peptide BEH C18 column, optionally with a particle size of 1.7 μm, a pore size of 300 Å, and a column temperature of 40° C. In some embodiments, the column has a height of 150 mm and/or a diameter of 2.1 mm. In such embodiments, a flow rate of 0.525 mL/min may be used.

In some embodiments, the analytical method utilizes a gradient of water and acetonitrile. The water may comprise a buffer such as NH₄HCO₃, optionally at a concentration of 10 mM. The buffer may be 10 mM NH₄HCO₃ at a pH of 8. In some embodiments, the analytical method comprises use of a first mobile phase that is a buffer of 10 mM NH₄HCO₃ in LC-MS grade water and a second mobile phase that is 90% HPLC grade CH₃CN in HPLC grade H₂O. In such embodiments, the analytical method may comprise an elution gradient of 35-75% B over 20 minutes.

### Purification Methods

The purifying step may comprise preparative liquid chromatography, tangential flow filtration, ion exchange, lyophilization, or a combination thereof.

Preparative liquid chromatography may be achieved in a one-dimensional purification strategy by reversed phase preparative HPLC. In some embodiments, the peptide is dissolved and subjected to decarboxylation and isoacyl rearrangement as described in Table 20A of Example 3. In a particular embodiment, the purification is carried out using NH₄OAc as a modifier in aqueous, acetonitrile (MeCN)-containing eluents. The peptide may be eluted with a gradient of increasing MeCN concentration. Suitably pure fractions may be combined, while less pure fractions may be recycled in further purification runs. An exemplary preparative liquid chromatography procedure is described in Table 20B of Example 3.

Concentration and buffer exchange of the fraction pool from the previous preparative HPLC may be performed in portions via ultrafiltration in a common tangential flow filtration unit. Tangential flow filtration may be carried out in two steps (Concentration I and Final Concentration) with an intermediate diafiltration with five volumes of purified water. In some embodiments, the concentrated product solution is filtrated into a retentate bag for storage until the following step. Exemplary parameters are described in Table 21 of Example 3.

The purified and concentrated material obtained from the previous ultrafiltration step may be homogenized. In some embodiments, ion exchange from ammonium to sodium is achieved by adding aqueous 1.0 M Na₂CO₃, followed by a filtration using a cellulose acetate membrane with a pore size of 0.2 μm. The final product may be isolated by lyophilization. Exemplary details of this procedure are described in Table 22 of Example 3.

A particular embodiment of the purification step is described in Example 3, which is applicable on a large synthetic scale.

In a further embodiment the purification can be performed as follows.

The peptides may be purified via high-performance liquid chromatography (HPLC).

The HPLC methods may comprise us of a Silicagel Basic C8 resin, optionally with a pore size of 20 nm or a particle size of 10 μm. The purification column may have a bed height of 25.5 cm. In some embodiments, the column has a bed height of 25.5 cm and a diameter of 5 cm, and a flow rate of 80 mL/min is used.

In some embodiments, the purification comprises use of a triethylammonium phosphate (TEAP) buffer. The TEAP buffer may be at a pH of 5.4 or 6.5. In some embodiments, the TEAP buffer has a pH of 5.4, and may be prepared at any volume by mixing triethylamine (TEA), H₃PO₄, and water to arrive at a ratio of 850 mL TEA:420 mL H₃PO₄: 100 L total volume.

Peptides may be purified by use of a gradient. The gradient may comprise use of a first mobile phase comprising TEAP (at pH 5.4 or 6.5) in purified water and a second mobile phase comprising TEAP in 80% CH₃CN. In some embodiments, the purification comprises a gradient of 60% of the first mobile phase/40% of the second mobile phase to 20% of the first mobile phase/80% of the second mobile phase over 80 minutes.

In some embodiments, peptides are purified through the use of consecutive chromatographic steps. For example, the purifying may comprise the use of hydrocarbon bonded silica as a stationary phase and a first mobile phase comprising TEAP and acetonitrile in a first step, followed by a second step with a second mobile phase comprising trifluoro acetic acid (TFA) and acetonitrile. The peptides may thus be purified through use of buffers comprising trifluoro acetic acid (TFA). The TFA may be at a concentration of 0.1%. Peptides may be purified by use of a gradient. The gradient may comprise use of a first mobile phase comprising 0.1% TFA in purified water and a second mobile phase comprising 0.1% TFA in 20% water/80% CH₃CN. In some embodiments, the purification comprises a gradient of 60% of the first mobile phase/40% of the second mobile phase to 20% of the first mobile phase/80% of the second mobile phase over 80 minutes.

### Salt Exchange

In some embodiments, the peptides are isolated as TFA or HCl salts. In order to exchange the TFA or HCl salt for a different salt (e.g., NH₄), a salt exchange protocol using a purification column may be used. Peptides may be purified by use of a gradient. The gradient may comprise use of a first mobile phase comprising 0.1 M NH₄HCO₃ pH 8 or pH 9 in purified water and a second mobile phase comprising 0.1 M NH₄HCO₃ pH 8 or pH 9 in 20% water and 80% CH₃CN. In some embodiments, the purification comprises a gradient of 60% of the first mobile phase/40% of the second mobile phase to 0% of the first mobile phase/100% of the second mobile phase over 45 minutes.

### V. Compositions Comprising CT-868

The present disclosure also relates to a composition comprising CT-868 in 0.1 M NH₄HCO₃ at pH 9-10. CT-868 may be soluble in a buffer at a concentration of up to about 20 g/L. In some embodiments, CT-868 is present in the composition at a concentration of 15 g/L or 20 g/L. In some embodiments, the composition comprises a 0.1 M NH₄HCO₃ buffer at pH 10 and CT-868 at a concentration of 15 g/L. The concentration of CT-868 in solution refers to the concentration of the free peptide in solution, not to the concentration of a salt form (e.g., the ammonium salt form) of the peptide in solution. The molecular weight of the free peptide (average mass) is 4610.2, and the monoisotopic mass (exact mass) is 4607.310.

The composition may be obtained by dissolving lyophilized CT-868 ammonium salt in 0.1 M NH₄HCO₃ pH 10.

In some embodiments, CT-868 is stable in the composition at room temperature or elevated temperature (e.g., 35° C.). CT-868 may be stable for 1, 2, 3, 6, 7, or more days in the composition.

### VI. Synthesis of Intermediate 2-((2-oxo-2-((2-(2-oxopiperidin-1-yl)ethyl)amino)ethyl)thio)acetic acid (III)

The synthesis of this building block may be carried out by the process illustrated in the following scheme (see also PCT International Publication WO 2024/264014, Examples 6 and 7).

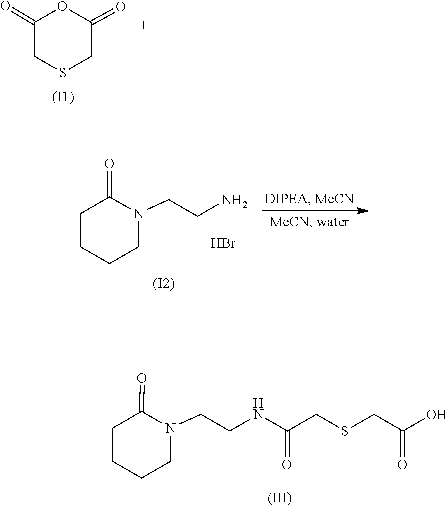

In some embodiments, the process comprises reacting 2-(2-Piperidon-1-yl)-ethylamine hydrobromide (I2) and thiodiglycolic anhydride (I1) in acetonitrile (MeCN) in the presence of DIPEA. The reaction may be conducted at temperatures ranging from 15-25° C. The resulting compound (III) may be obtained in high yields and purity by filtering it from the reaction mixture, washing the filter cake with MeCN, and subsequently drying.

The 2-(2-Piperidon-1-yl)-ethylamine hydrobromide intermediate of Formula (I2)

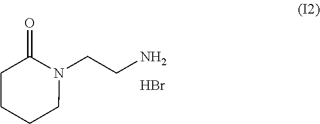

may be prepared according to the following scheme (see also PCT International Publication WO 2024/264014, Example 3).

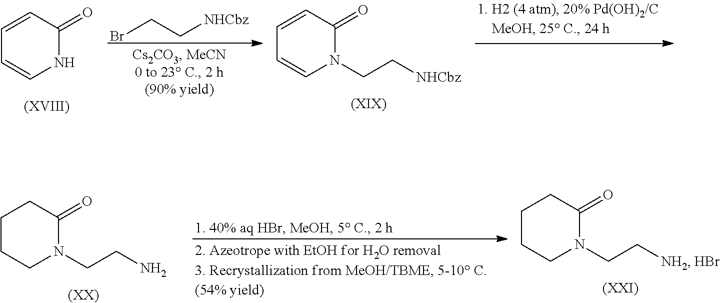

In some embodiments, the intermediate 2-(2-Piperidon-1-yl)-ethylamine hydrobromide of formula (I2)

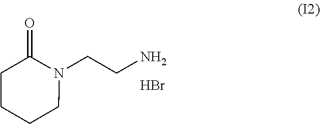

is prepared by reacting 2-piperidone (I3) with bromoacetonitrile (I4) in the presence of a strong base, followed by hydrogenating of the cyano group, protecting the amino group with a Boc group, and transferring the amine (I6) into the hydrobromide intermediate (I2) by adding HBr.

The 2-(2-Piperidon-1-yl)-ethylamine hydrobromide intermediate of formula (I2)

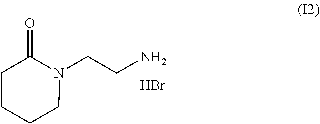

may be prepared following the general scheme.

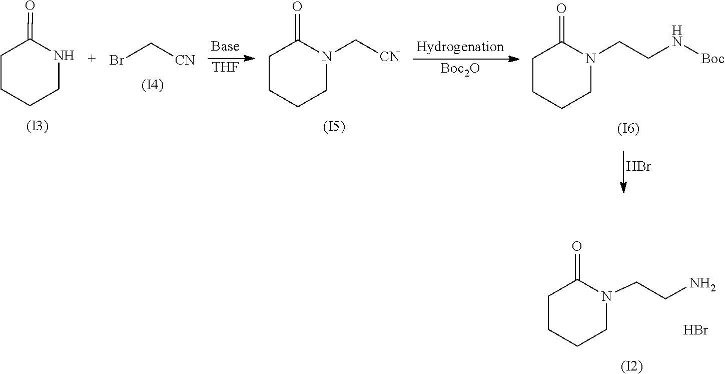

The first step may comprise reacting 2-piperidone (I3) with bromoacetonitrile (I4). This reaction may be conducted in the presence of a strong base, which may be selected from alkali alcoholates such as lithium, sodium, or potassium tert-butylate, or alkali hydrides such as sodium hydride. A suitable organic solvent, such as tetrahydrofuran, may be employed. The reaction temperature may range from −20° C. to 40° C. (e.g., a range of 10° C. to 30° C.). A quaternary ammonium salt, for instance, tetrabutylammonium bromide (TBAB), may be added to serve as a phase transfer catalyst. Following the reaction, the reaction mixture can be quenched by adding water. Extracting intermediate (I5) from the reaction mixture may be performed using a suitable organic solvent, such as toluene. While intermediate (I5) may be isolated by removing the solvent, in a particular embodiment, it is directly transferred to the subsequent hydrogenation reaction, typically after a solvent swap to, for example, methanol or tetrahydrofuran.

The hydrogenation reaction may be performed using classical hydrogenation catalysts, which may be selected from Raney Ni, Pd/C, or PtO₂. In some embodiments, this reaction applies a hydrogen pressure of 2 to 10 bar at a temperature range of 0° C. to 40° C. Specifically, the reaction employing Raney Ni with a hydrogen pressure of 7 to 9 bar at 20° C. to 30° C. may be used. A suitable reaction medium for this step is a saturated solution of ammonia in an appropriate organic solvent, such as tetrahydrofuran or methanol. The resulting 2-(2-Piperidon-1-yl)-ethylamine intermediate can, after filtering off the catalyst and a potential solvent swap, be directly transferred to the subsequent bocylation step. This bocylation, involving Boc-anhydride (Boc₂O), may proceed at an ambient temperature of 20° C. to 30° C., leading to the formation of the Boc-protected 2-(2-Piperidon-1-yl)-ethylamine intermediate (I6). Intermediate (I6) may then be isolated by removing the solvent, potentially followed by crystallizing, using, for example, ethyl acetate.

The formation of intermediate (I2) may be achieved by adding HBr in an organic solvent, such as ethyl acetate, to a solution of intermediate (I6), in some embodiments also in ethyl acetate as the organic solvent. The reaction temperature may be between 10° C. and 40° C. (e.g., a range of 20° C. to 30° C.). The resulting 2-(2-Piperidon-1-yl)-ethylamine hydrobromide of formula (I2) may then be filtered off, washed, and dried.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In some embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Solid-Phase Synthesis of Peptide

The net molecular weight of the peptide (MWₙₑₜ) is 4610.2 g/mol. The synthesis described herein was carried out on a molar scale of 0.13 mol. The theoretical yield (net peptide) is 599.3 g (Molar scale×MWₙₑₜ, i.e. 0.13 mol×4610.2 g/mol). The molecular weight of the protected peptide (MWprot) is 6120.7 g/mol. The molecular weight of the functional group (MWFG; 2-((2-(2-oxopiperidin-1-yl)ethylcarbamoyl)methylthio) acetic acid (III)) is 239.0 g/mol.

### Preparation of the Resin

213.1 g (0.61 mmol/g) of Tricyclic Amide Linker Resin was added to the reactor. The resin was stirred in approximately 10 ml of DMF per 1 g of resin (i.e., 2131 mL DMF). Without draining the reactor, stirring was stopped and the resin was allowed to swell in DMF for a minimum of two hours. The reactor was then drained.

### General Synthetic Procedure

Washing steps were designed to remove remaining deprotection solution, coupling reagents, and additives between process steps. Before each wash, the reactor was completely drained (i.e., until the flow into the waste accumulation vessel has stopped). Enough solvent was added to the reactor so that the fluid reached 1.5 to 2.0 times the resin bed height, and the mixture was stirred for a minimum of three minutes (starting after solvent addition is complete and stirring is started). After a minimum of 3 minutes, the stirrer was stopped and the reactor was drained completely.

Fmoc deprotection was performed by stirring the resin in enough Fmoc deprotection solution so that the fluid reaches 1.5 to 2.0 times the resin bed height. Two deprotection treatments were performed. The first Deprotection stirring time was 15±1 minutes. The second deprotection stirring time was 35±1 minutes. Time starts after solvent addition is complete and stirring is started. The Fmoc Deprotection Solution is 20% Piperidine in DMF.

For coupling/recoupling, a single-use HDPE bucket was used. Recoupling uses the same method as coupling, with 50% of the amino acid derivative and coupling reagents.

For all couplings except for Cycle 35 (i.e., addition of 2-((2-(2-oxopiperidin-1-yl)ethylcarbamoyl)methylthio) acetic acid (III)), a DIC OxymaPure™ procedure was used. The amino acid and OxymaPure™ were placed in an HDPE bucket and dissolved together in DMF. DIC was then added (1st Addition) using an addition funnel and, once addition was complete, the mixture was stirred for 20-30 minutes to pre-activate the amino acid before addition to the reactor.

Once pre-activation was complete, the amino acid solution was added to the reactor. The reaction mixture was then stirred. If necessary, a minimum volume of DMF was added to the reactor in order to ensure that the resin bed was fluid. 20 minutes after adding the amino acid solution to the reactor, DIC was added (2nd Addition). The reaction time for the coupling was 1 to 24 hours. Recoupling was performed for Cycle 1 and Cycle 19.

For Cycle 35, which immediately followed cycle 34, a DIPEA/TBTU procedure was used. The carboxylic acid and TBTU were placed in an HDPE bucket and dissolved together in the required volume of DMF. DIPEA was then added using an addition funnel and, once addition was complete, the mixture was stirred for three minutes to pre-activate the amino acid before addition to the reactor. Once pre-activation was complete, the amino acid solution was added to the reactor. The reaction mixture was then stirred. If necessary, a minimum volume of DMF was added to the reactor in order to ensure that the resin bed was fluid. The reaction time for coupling was 1 to 3 hours.

In-process monitoring was carried out following Fmoc deprotection, amino acid coupling, and acetylation by performing the Ninhydrin and TNBS colorimetric tests. For cycles following the addition of Fmoc-Pro-OH, the chloranil colorimetric test was used instead.

For Fmoc deprotection, the required test was performed after the completion of the washes following Fmoc deprotection. A drained sample was removed from the reactor and placed in a disposable syringe or filter. The sample was washed eight times alternating between DMF and IPA. A small amount of this sample was used for performing the tests. In addition, residual Fmoc testing was performed for Cycle 19.

For testing amino acid coupling and re-coupling, once the minimum coupling time was reached, a small liquid sample was removed from the reactor and placed in a disposable syringe or filter. The sample was washed eight times alternating between DMF and IPA and the required colorimetric tests were performed. The results of these tests were expected to be negative. If any test result was positive, the coupling was allowed to continue up to the maximum coupling time, with re-sampling and testing periodically to monitor the reaction.

For in-process monitoring following capping, the required test was performed after the completion of acetylation. A drained sample was removed from the reactor, and a small amount of this sample was used for performing the test. The sample was placed in a disposable syringe or filter. The sample was washed eight times alternating between DMF and IPA, and the required colorimetric tests were performed. If the result of a test was slightly positive or inconclusive, the result was confirmed by performing a side-by-side micro-capping and repeating the test as described above. The results of these tests must be negative.

Capping was mandatory for Cycle 1 and Cycle 19.

### Synthetic Procedure

Washing and Fmoc deprotection were performed as follows:
1. Perform DMF wash for a minimum of three minutes.
2. Treat resin with 20% piperidine in DMF for 15+1 minutes.
3. Treat resin with 20% piperidine in DMF for 35+1 minutes.
4. Perform each wash for a minimum of three minutes each. Washes 1, 3, and 4: DMF; wash 2: IPA.
5. Perform Ninhydrin and TNBS testing.
6. Perform two DMF washes, each for a minimum of three minutes.

Each coupling other than cycle 35—which immediately follows cycle 34—was performed as follows. Details regarding reagent and solvent amounts, temperatures, and reaction/stirring times can be found in Tables 3 and 4.

10. Dissolve Fmoc-protected amino acid and 55.4 g OxymaPure™ in DMF in an HDPE bucket equipped with a magnetic stir bar and placed on a magnetic stir plate.
11. Record the temperature of the amino acid solution before DIC addition, after DIC addition, and after pre-activation is complete. Add 53 ml of DIC (1st Addition) using an addition funnel to the amino acid solution. After addition, stir for 20-30 minutes to pre-activate.
12. Add the activated amino acid solution to the reactor. If necessary, add enough additional DMF to make the resin bed fluid. 20 minutes after adding the amino acid solution to the reactor, add 29 ml of DIC (2^{nd} Addition). The coupling time for this reaction is 1-24 hours.
13. After 1 hour of coupling, monitor the reaction periodically using the Ninhydrin and TNBS colorimetric tests until negative results are achieved or until the 24-hour maximum coupling time has been reached. If the results are negative, stop the reaction, record the coupling stop time, and then proceed with Recoupling (where appropriate). After 24 hours, drain the reactor, record the coupling stop time, and perform a final set of colorimetric tests.
14. Fill the reactor to a level 1.5 to 2.0 times the resin bed height with DMF, stir for a minimum of 3 minutes, and then drain the reactor.

### Re-Coupling: Steps 6-9 are Performed for Cycles 1 and 19 Only

15. Dissolve Fmoc-protected amino acid and 27.7 OxymaPure™ in DMF in an HDPE bucket equipped with a magnetic stir bar and placed on a magnetic stir plate.
16. Record the temperature of the amino acid solution before DIC addition, after DIC addition, and after pre-activation is complete. Add 27 ml of DIC (1 st Addition) using an addition funnel to the amino acid solution. After addition, stir for 20-30 minutes to pre-activate.
17. Add the activated amino acid solution to the reactor. If necessary, add enough additional DMF to make the resin bed fluid. 20 minutes after adding the amino acid solution to the reactor, add 14 ml of DIC (2^{nd} Addition). The coupling time for this reaction is 1-24 hours.
18. After 1 hour of coupling, monitor the reaction periodically using the Ninhydrin and TNBS colorimetric tests until negative results are achieved or until the 24-hour maximum coupling time has been reached. If the results are negative, stop the reaction, record the coupling stop time, and proceed with Capping. After 24 hours, drain the reactor, record the coupling stop time, and perform a final set of colorimetric tests. If the results are strongly positive, proceed with Recoupling (request an additional set of pages from QA). If the results are slightly positive or negative, proceed with Capping.

### Capping: Steps 10-18 are Performed for Cycles 1 and 19 Only

19. Wash the resin twice. For each wash, fill the reactor to a level 1.5 to 2.0 times the resin bed height with DMF, stir for a minimum of 3 minutes, and then drain the reactor.
20. Prepare the 20% DIPEA in DMF solution by adding 23 ml of DIPEA and 92 ml of DMF to a glass beaker or flask. Stir the mixture for a minimum of 1-3 minutes.
21. Prepare the 20% Ac₂O in DMF solution by adding 123 ml of Ac₂O and 492 ml of DMF to a glass beaker or flask. Stir the mixture for a minimum of 1-3 minutes.
22. Add enough DMF to the reactor to make the resin bed fluid, and begin stirring the resin. Add the prepared 20% DIPEA in DMF solution to the reactor.
23. Add the prepared 20% Ac₂O in DMF solution to the reactor
24. Stir for 15-30 minutes (time starts when addition of the 20% Ac₂O in DMF solution is complete), and then drain the reactor.
25. Wash the resin 5 times. For each wash, fill the reactor to a level 1.5 to 2.0 times the resin bed height with the listed solvent, stir for a minimum of 3 minutes, and then drain the reactor. Washes 1 and 3-5: DMF; wash 2: IPA.
26. Perform Ninhydrin and TNBS colorimetric testing. The results must be negative.
27. Perform washes for a minimum of three minutes each. Washes 1 and 3: DMF; wash 2: IPA.

TABLE 3
Details of Solid-Phase Synthesis Coupling Steps

| | | | | Step 2 | | | | | |
|---|---|---|---|---|---|---|---|---|---|
| | | | | Temp | Temp | | | | |
| | | Step 1 | | before | after | | Temp | | Step 4 |
| | | | DMF | DIC | DIC | | after pre- | Step 3 | Total |
| | | Amount | used | addition | addition | Stirring | activation | Additional | coupling |
| Cycle | Coupled Unit | used | (mL) | (° C.) | (° C.) | time | (° C.) | DMF | time |
| 1 | Fmoc- | 205.9 g | 1000 | 21.6 | 22.0 | 20 | 26.0 | 200 | 15:43 |
| | Lys(Palmitoyl-Glu- | | | | | minutes | | | |
| | OtBu)-OH | | | | | | | | |
| 2 | Fmoc-Ser(tBu)-OH | 99.7 g | 500 | 23.1 | 26.0 | 25 | 33.3 | 200 | 17:17 |
| | | | | | | minutes | | | |
| 3 | Fmoc-Pro-Pro-OH | 113.0 g | 500 | 22.2 | 22.7 | 25 | 31.2 | 200 | 1:18 |
| | | | | | | minutes | | | |
| 4 | Fmoc-Pro-OH | 87.7 | 500 | 22.3 | 23.1 | 25 | 31.2 | 200 | 1:34 |
| 5 | Fmoc-Ala-OH H₂O | 85.6 | 600 | 22.6 | 23.1 | 20 | 32.1 | 200 | 2:23 |
| 6 | Fmoc-Ser(tBu)-Gly- | 114.5 | 800 | 22.5 | 23.2 | 25 | 31.1 | 200 | 2:40 |
| | OH | | | | | | | | |
| 7 | Fmoc-Ser(tBu)-OH | 99.7 | 500 | 22.1 | 22.7 | 25 | 32.7 | 200 | 1:30 |
| 8 | Fmoc-Pro-OH | 87.7 | 500 | 23.1 | 23.7 | 25 | 33.7 | 200 | 2:20 |
| 9 | Fmoc-Gly-Gly-OH | 92.1 | 500 | 21.0 | 21.8 | 25 | 38.7 | 200 | 1:30 |
| 10 | Fmoc-Ala-OH H₂O | 85.6 | 500 | 22.2 | 23.1 | 25 | 33.0 | 300 | 1:54 |
| 11 | Fmoc-Leu-OH | 91.9 | 800 | 24.9 | 25.2 | 25 | 31.0 | 200 | 2:50 |
| 12 | Fmoc-Leu-OH | 91.9 | 500 | 23.0 | 23.7 | 25 | 30.9 | 300 | 1:30 |
| 13 | Fmoc-Trp(Boc)-OH | 136.9 | 600 | 27.2 | 27.9 | 25 | 34.3 | 300 | 2:04 |
| 14 | Fmoc-Asn(Trt)-OH | 155.1 | 800 | 27.6 | 28.2 | 25 | 33.8 | 300 | 1:47 |
| 15 | Fmoc-Val-OH | 88.2 | 800 | 25.0 | 26.2 | 25 | 31.2 | 300 | 2:45 |
| 16 | Fmoc-Phe-OH | 100.7 | 800 | 29.7 | 30.1 | 22 | 36.1 | 200 | 1:45 |
| 17 | Fmoc-Glu(OtBu)- | 115.3 | 800 | 26.3 | 26.6 | 20 | 33.6 | 200 | 1:40 |
| | OH H₂O | | | | | | | | |
| 18 | Fmoc-Aib-OH | 84.6 | 800 | 26.0 | 27.2 | 25 | 32.3 | 300 | 3:18 |
| 19 | Fmoc-Ala-OH H₂O | 85.6 | 800 | 26.9 | 27.4 | 20 | 33.9 | 100 | 2:46 |
| 20 | Fmoc-Ala-OH H₂O | 85.6 | 800 | 24.9 | 25.8 | 25 | 36.6 | 300 | 3:36 |
| 21 | Fmoc-Gln(Trt)-OH | 158.8 | 700 | 27.8 | 28.4 | 25 | 35.3 | 300 | 2:56 |
| 22 | Fmoc-Lys(Boc)-OH | 121.8 | 800 | 28.1 | 28.5 | 20 | 33.5 | 200 | 2:15 |
| 23 | Fmoc-Asp(OMpe)- | 114.3 | 800 | 28.1 | 28.7 | 20 | 35.2 | 200 | 2:15 |
| | OH | | | | | | | | |
| 24 | Fmoc-Leu-OH | 91.9 | 600 | 22.7 | 23.9 | 25 | 32.0 | 300 | 1:30 |
| 25 | Fmoc-Tyr(tBu)-OH | 119.5 | 600 | 23.3 | 24.4 | 25 | 31.7 | 300 | 1:30 |
| 26 | Fmoc-Ile-OH | 91.9 | 600 | 24.1 | 24.9 | 25 | 32.3 | 200 | 2:55 |
| 27 | Fmoc-Ser(tBu)-OH | 99.7 | 600 | 21.0 | 21.9 | 25 | 30.8 | 300 | 3:00 |
| 28 | Fmoc-Tyr(tBu)-OH | 119.5 | 800 | 24.4 | 24.8 | 20 | 31.3 | 200 | 4:52 |
| 29 | Fmoc-Asp(OMpe)- | 114.3 | 600 | 21.2 | 22.1 | 25 | 28.8 | 300 | 2:26 |
| | OH | | | | | | | | |
| 30 | Fmoc-Thr(tBu)- | 136.4 | 800 | 28.0 | 28.5 | 20 | 33.8 | 200 | 3:20 |
| | Ser(Ψ(Me,Me)pro)- | | | | | | | | |
| | OH | | | | | | | | |
| 31 | Fmoc-Phe-OH | 100.7 | 600 | 21.9 | 22.7 | 25 | 30.1 | 300 | 2:21 |
| 32 | Fmoc-Thr(tBu)-OH | 103.3 | 800 | 23.0 | 23.5 | 20 | 30.2 | 200 | 5:07 |
| 33 | Fmoc-Gly-OH | 77.3 | 600 | 19.0 | 19.9 | 25 | 26.9 | 300 | 3:04 |
| 34 | Fmoc-Glu(OtBu)- | 115.3 | 600 | 18.2 | 19.2 | 25 | 27.9 | 300 | 2:46 |
| | OH H₂O | | | | | | | | |

TABLE 4
Details of Solid-Phase Synthesis Re-Coupling Steps

| | | | | Step 7 | | | | | | |
|---|---|---|---|---|---|---|---|---|---|---|
| | | | | Temp | Temp | | | | | Step |
| | | | | before | after | | Temp | | Step 9 | 15 |
| | | Step 6 | | DIC | DIC | Total | after pre- | Step 8 | Total | Total |
| | Coupled | Amount | | addition | addition | stirring | activation | Additional | coupling | stirring |
| Cycle | Unit | used | DMF | (° C.) | (° C.) | time | (° C.) | DMF | time | time |
| 1 | Fmoc- | 103.0 | 500 | 20.2 | 20.5 | 25 | 24.4 | 200 mL | 2:26 | 20 |
| | Lys(Palmitoyl- | | | | | mins | | | | mins |
| | Glu-OtBu)- | | | | | | | | | |
| | OH | | | | | | | | | |
| 19 | Fmoc-Ala-OH | 42.8 | 350 | 25.6 | 26.2 | 20 | 30.6 | 300 | 1:41 | 20 |
| | H₂O | | | | | | | | | |

The coupling procedure for cycle 35, which immediately follows cycle 34 in Table 3, was as follows: 76.0 g of 2-(2-piperidon-1-yl)-ethylcarbamoylmethylthioacetic acid and 73.1 g of TBTU were dissolved in 760 ml of DMF in an HDPE bucket equipped with a magnetic stir bar and placed on a magnetic stir plate. The temperatures of the TBTU solution before DIPEA addition (15.2° C.), after DIPEA addition (20.1° C.), and after pre-activation is complete (32.1° C.) were recorded. 136 ml of DIPEA was then added to the TBTU solution using an addition funnel. After addition, the mixture was stirred for 3 minutes to pre-activate. The activated solution was then added to the reactor. 300 mL DMF was added to make the resin bed fluid.

After one hour of coupling, the reaction was monitored periodically using the Ninhydrin and TNBS colorimetric tests until negative results were achieved or until the 3-hour maximum coupling time has been reached. The total coupling time was 1:25.

For the final washing, the reactor was filled to a level 1.5 to 2.0 times the resin bed height with IPA, stirred for a minimum of 3 minutes, and then drained. This was repeated eight times. The weight gain of the resin was 715.30 g (928.4 g weight of peptidyl-resin-213.1 g weight of starting resin), indicating a 93.6% yield of peptidyl-resin.

### Example 2: Purification and Salt Exchange

This Example describes a series of experiments designed to evaluate an improved purification strategy for CT-868 peptide and conversion to ammonium salt form. Additionally, experiments were performed to evaluate the conversion of hydrochloride (HCl) salt form of CT-868 to ammonium (NH₄⁺) salt form and stability of the new salt form. Prior to these experiments, CT-868 peptide was produced as a hydrochloride salt. Due to the nature of the peptide (net negatively charged, acidic), it has been proposed that varying the pH of mobile phases and changing the salt form to ammonium would be beneficial. The hydrophobicity of the peptide poses challenges for solubility, which can also be overcome by varying the pH and modifiers of the mobile phases. Select equipment used in these experiments are provided below.
ModColumn, 5 cm column diameter, 25.5 cm column length
Waters ACQUITY UHPLC Instrument
Waters ACQUITY Peptide BEH C8 Column
Waters ACQUITY Peptide BEH C18 Column
GORE Lyoguard Cup Feeze-Drying Trays
LSI Tray Lyophilizer (28 L)

First, an improved purification strategy for CT-868 peptide and the conversion of hydrochloride salt to ammonium salt was evaluated. Analytical UHPLC System 1 (Waters ACQUITY Peptide BEH C8 column) and analytical UHPLC System 2 (Waters ACQUITY Peptide BEH C18 column) were used to determine purity of individual fractions and subsequent main pools for Stage 4 primary purification, Stage 5 secondary purification, and Stage 6 salt exchange purification. In addition, 10 mM NH₄HCO₃ solution at pH 8, pH 9, and pH 10 was analyzed by analytical UHPLC System 2 (C18). A comparison of C8 versus C18 column results for Stage 4 Run 4-1 as the representative stage/run and as well as the overlay chromatogram of the analytical UHPLC of 10 mM NH₄HCO₃ solution at pH 8, pH 9, and pH 10 are presented in Table 5 below.

TABLE 5
In-Process Control Analytical UHPLC Method Comparison

| | System 1 - C8 Column | | System 2 - C18 Column | |
|---|---|---|---|---|
| | UHPLC Conditions | | UHPLC Conditions | |
| Instrument | Waters ACQUITY UHPLC | | Waters ACQUITY UHPLC | |
| Column | Waters ACQUITY | | Waters ACQUITY | |
| | Peptide BEH C8 | | Peptide BEH C18 | |
| Column Size | 2.1 mm × 150 mm | | 2.1 mm × 150 mm | |
| Particle Size | 1.7 | μm | 1.7 | μm |
| Pore Size | 300 | Å | 300 | Å |
| Mobile | 10 mM LC-MS Grade | | 10 mM LC-MS Grade | |
| Phase A | NH₄HCO₃ | | NH₄HCO₃ | |
| Mobile | 90% HPLC Grade CH₃CN | | 90% HPLC Grade CH₃CN | |
| Phase B | in HPLC Grade H₂O | | in HPLC Grade H₂O | |
| H2O Gradient | 35 →75% Mobile Phase | | 35 →75% Mobile Phase | |
| | B in 20 minutes | | B in 20 minutes | |
| Flow rate | 0.525 | mL/minute | 0.525 | mL/minute |
| Absorbance | 220 | nm | 220 | nm |
| Column | 40° | C. | 40° | C. |
| Temperature | | | | |
| Autosampler | 8° | C. | 8° | C. |
| temperature | | | | |

Three sublots of lyophilizate solution were prepared for three Stage 4 primary purifications (TEAP pH 5.4, TEAP pH 6.5, and Ammonium Phosphate mobile phase systems) using lyophilized peptide (Stage 4, TEAP pH 2.3). For each sublot, i.e., 4-1, 4-2, and 4-3, 3.0 g of lyophilized 4-6 main pool (Stage 4, TEAP pH 2.3) from CT-868 were dissolved in 300 mL of 0.1 M NH₄HCO₃ pH 8. Each solution was stirred with a stir bar in a beaker on a stir plate for 30 minutes. Prior to column loading, the lyophilizate solutions were filtered using a 0.45 μm disposable filter.

### Stage 4 Primary Purification

### TEAP pH 5.4 Mobile Phase System

The Stage 4 Primary Purification (TEAP pH 5.4 Mobile Phase System) Preparative HPLC Equipment and Mobile Phases are summarized below:
Column type: ModColumn, 5 cm diameter, 25.5 cm length Pumping skid: Azura UVD 2.1 S
Packing material: Silicagel Basic C8 Manufacturer: YMC
Pore size: 20 nm Particle size: 10 μm
Mobile Phase A (MPA): TEAP pH 5.4 in purified water Mobile Phase B (MPB): TEAP pH 5.4 in 80% CH₃CN Mobile Phase C (MPC): TEAP pH 5.4 in 30% CH₃CN Mobile Phase X (MPX): 80% CH₃CN
Flow rate: 80 mL/min Detector wavelength: 230 nm

Column equilibration was performed with 1.0 L of MPC and 300 mL of the solution was applied to the column, followed by 1.0 L of MPC. The lyophilized peptide was eluted from the column with a gradient of 40→80% MPB in 80 minutes. After the lyophilized peptide had eluted completely, the column was washed with 1.0 L of MPX. Individual fractions with a volume of 75-150 mL were collected and analyzed for purity by analytical UHPLC System 1 and System 2. Pooling of the selected main pool fractions into a main pool was performed according to the acceptance criteria for purity ≥95%, and pooling of the sidecut fractions into a sidecut pool was performed according to the acceptance criteria for purity ≥70%. Main pool 4-1 was stored in a refrigerator at ≤8° C. until further processing. Front and rear sidecut pools 4-1 were discarded.

### TEAP pH 6.5 Mobile Phase System

The conditions for the Stage 4 Primary Purification (TEAP pH 6.5 Mobile Phase System) were as follows:
Mobile Phase A (MPA): TEAP pH 6.5 in purified water
Mobile Phase B (MPB): TEAP pH 6.5 in 80% CH₃CN
Mobile Phase C (MPC): TEAP pH 6.5 in 30% CH₃CN
Mobile Phase X (MPX): 80% CH₃CN.
Flow rate: 80 mL/min
Detector wavelength: 230 nm

Column equilibration was performed with 1.0 L of MPC, and 300 mL of the lyophilizate solution was applied to the column followed by 1.0 L of MPC. The lyophilized peptide was eluted from the column with a gradient of 40-80% MPB in 80 minutes. After the lyophilized peptide had eluted completely, the column was washed with 1.0 L of MPX. Individual fractions of 125 mL volume were collected and analyzed for purity by analytical UHPLC System 1 and System 2. Pooling of the selected main pool fractions into a main pool was performed according to the acceptance criteria for purity ≥95%, and pooling of the sidecut fractions into a sidecut pool was performed according to the acceptance criteria for purity ≥70%. Main pool 4-2 was stored in a refrigerator at ≤8° C. until further processing. Front and rear sidecut pools 4-2 were discarded.

### Ammonium Phosphate Mobile Phase System

The conditions for the Stage 4 Primary Purification (Ammonium Phosphate Mobile Phase System) were as follows:
Mobile Phase A (MPA): Ammonium Phosphate in purified water
Mobile Phase B (MPB): Ammonium Phosphate in 50% CH₃CN.
Mobile Phase C (MPC): Ammonium Phosphate in 30% CH₃CN Mobile Phase X (MPX): 80% CH₃CN
Flow rate: 80 mL/min
Detector wavelength: 230 nm

Column equilibration was performed with 1.0 L of MPC, and 300 mL of the lyophilizate solution was applied to the column followed by 1.0 L of MPC. A gradient of 40+80% MPB in 80 minutes was applied to the column but no product (lyophilized peptide) eluted. The product did elute during the column wash with 1.0 L of MPX.

### Results

Results are summarized in Tables 6-8 below.

TABLE 6
Stage 4 Primary Purification UHPLC System 2 (C18) Analysis Results

| | | | Main Pool | Front | Rear | |
|---|---|---|---|---|---|---|
| | | Main Pool | Purity (%) | Sidecut | Sidecut | |
| Stage 4 Mobile | Run | Fraction #/ | by System | Fraction #/ | Fraction #/ | Fractions |
| Phase System | # | Volume | 2 (C18) | Volume | Volume | Discarded |
| TEAP pH 5.4 | 1-Apr | 2-4/ | 96.8 | 1/ | 5/ | None |
| | | 0.25 L | | 0.15 L | 0.075 L | |
| TEAP pH 6.5 | 2-Apr | 3-7/ | 99.6 | 1, 2/ | 8/ | None |
| | | 0.625 L | | 0.25 L | 0.15 L | |
| Ammonium | 3-Apr | None/ | None/ | None/ | None/ | None |
| Phosphate¹ | | N/A | N/A | N/A | N/A | |

¹High pressure stopped at 75% MPB during gradient run. Product never came out throughout the gradient run. 100 mL fractions were collected when the column was washed with MPX. MPB:0.1M Ammonium Phosphate buffer did not dissolve in 80% CH₃CN, therefore 50% CH₃CN was used instead.

TABLE 7
Run 4-1; TEAP pH 5.4; Fractions and Main Pool
Purity by System 1 (C8) and System 2 (C18)

| Fractions | C8 Purity (%) | C18 Purity (%) | Fraction volume (mL) |
|---|---|---|---|
| 1 | 84.5 | 81.2 | 150 |
| 2 | 96.9 | 97.0 | 100 |
| 3 | 95.4 | 95.5 | 75 |
| 4 | 94.9 | 95.1 | 75 |
| 5 | 83.8 | 88.3 | 75 |
| MP | 97.1 | 96.8 | |

Main pool were fractions 2-4.
Main pool: greater than or equal to 95%.
Sidecuts greater than or equal to 70%.
Buffers used:
A: TEAP, pH 5.4
B: TEAP, pH 5.4 in 80% CH₃CN
C: TEAP, pH 5.4 in 30% CH₃CN
X: 80% CH₃CN
Gradient: 40-80% B in 80 minutes

TABLE 8
Run 4-2 (TEAP pH 6.5) Fractions and Main Pool
Purity by System 1 (C8) and System 2 (C18)

| Fractions | C8 Purity (%) | C18 Purity (%) | Fraction volume (mL) |
|---|---|---|---|
| 1 | 81.1 | 81.9 | 125 |
| 2 | 94.0 | 93.6 | 125 |
| 3 | 99.0 | 99.5 | 125 |
| 4 | 98.2 | 99.3 | 125 |
| 5 | 99.1 | 99.5 | 125 |
| 6 | 98.9 | 99.0 | 125 |
| 7 | 95.7 | 97.0 | 125 |
| 8 | 76.2 | 75.4 | 150 |
| MP | 97.6 | 99.6 | |

Main pool were fractions 3-7
Main pool: greater than or equal to 95%.
Sidecuts greater than or equal to 70%.
Buffers used:
A: TEAP, pH 6.5
B: TEAP, pH 6.5 in 80% CH₃CN
C: TEAP, pH 6.5 in 30% CH₃CN
X: 80% CH₃CN
Gradient: 40-80% B in 80 minutes

The purification using 0.1M Ammonium phosphate was not completed due to pressurization issues.

### Stage 5 Secondary Purification (0.1% TFA Mobile Phase System)

The conditions for the Stage 5 Secondary Purification (0.1% TFA Mobile Phase System) were as follows:
Mobile Phase A (MPA): 0.1% TFA in purified water.
Mobile Phase B (MPB): 0.1% TFA in 80% CH₃CN
Mobile Phase C (MPC): 0.1% in 30% CH₃CN
Mobile Phase X (MPX): 80% CH₃CN
Flow rate: 80 mL/min Detector wavelength: 230 nm

Column equilibration was performed with 1.0 L of MPC. Main pools 4-1 and 4-2 (875 mL total) from Stage 4 primary purification were diluted 1:1 (v/v) with purified water and applied to the column, followed by 1.0 L of MPC. The product was eluted from the column with a gradient of 40→80% MPB in 80 minutes. After the product had eluted completely, the column was washed with 1.0 L of MPX. Individual fractions of 75 mL volume were collected and analyzed for purity by analytical UHPLC System 1 and System 2. Pooling of the selected main pool fractions into a main pool was performed according to the acceptance criteria for purity ≥96.0%, and pooling of the sidecut fractions into a sidecut pool was performed according to the acceptance criteria for purity ≥70%. Main pool 5-1 was stored in a refrigerator at ≤8° C. until further processing. Front and rear sidecut pools 5-1 were discarded.

Results are shown in Tables 9 and 10 below.

TABLE 9
Stage 5 Secondary Purification UHPLC System 2 (C18) Analysis Results

| | | | | Front | Rear | |
|---|---|---|---|---|---|---|
| | | Main Pool | Main Pool | Sidecut | Sidecut | |
| Stage 5 Mobile | Run | Fraction #/ | Purity (%)by | Fraction #/ | Fraction #/ | Fractions |
| Phase System | # | Volume | System 2 (C18) | Volume | Volume | Discarded |
| 0.1% TFA | 5-1 | 3-5/ | 98.4 | 2/ | 6-8/ | 1 |
| | | 0.225 L | | 0.10 L | 0.225 L | |

TABLE 10
Run 5-1 (0.1% TFA) fractions and main pool
purity by System 1 (C8) and System 2 (C18)

| Fractions | C8 Purity (%) | C18 Purity (%) | Fraction volume (mL) |
|---|---|---|---|
| 1 | 24.6 | 29.1 | 150 |
| 2 | 69.8 | 89.9 | 100 |
| 3 | 92.0 | 98.3 | 75 |
| 4 | 93.6 | 99.0 | 75 |
| 5 | 91.2 | 97.5 | 75 |
| 6 | 89.3 | 93.7 | 75 |
| 7 | 91.6 | 91.6 | 75 |
| 8 | 81.8 | 92.0 | 75 |
| MP | 96.9 | 98.4 | |

Main pool: greater than or equal to 96.0%; Sidecuts greater than or equal to 70%.
Main pool: fractions 3-5 of C18
Buffers used:
A: 0.1% TFA
B: 0.1% TFA in 80% CH₃CN
C: 0.1% TFA in 30% CH₃CN
X: 80% CH₃CN
Gradient: 40-80% B in 80 minutes

### Stage 6 Salt Exchange Purification

0.1 M NH₄HCO₃ pH 8 Mobile Phase System

The conditions for the Stage 6 Salt Exchange (0.1 M NH₄HCO₃ pH 8 Mobile Phase System) were as follows:
Mobile Phase A (MPA): 0.1 M NH₄HCO₃ pH 8 in purified water
Mobile Phase B (MPB): 0.1 M NH₄HCO₃ pH 8 in 80% CH₃CN
Mobile Phase C (MPC): 0.1% TFA
Mobile Phase X (MPX): 80% CH₃CN Flow rate: 80 mL/min
Detector wavelength: 230 nm

Column equilibration was performed with 1.0 L of MPC. One half of main pool 5-1 (112.5 mL) from Stage 5 secondary purification was diluted 1:1 (v/v) with purified water and applied to the column, followed by two consecutive applications of 1.0 L of MPC. The product was eluted from the column with a gradient of 40-100% MPB in 45 minutes. After the product had eluted completely, the column was washed with 1.0 L of MPX. Individual fractions of 50 mL volume were collected and analyzed for purity by analytical UHPLC System 1 and System 2. Pooling of the selected main pool fractions into a main pool was performed according to the acceptance criteria for purity ≥98.0%, and pooling of the sidecut fractions into a sidecut pool was performed according to the acceptance criteria for purity ≥70%. Refer to Table 3 in Section 5.2.3 of this report for the main pool purity results by System 2. Main pool 6-1 was stored in a refrigerator at ≤80° C. until further processing. Front sidecut pool 6-1 was discarded. Subsequently, main pool 6-1 was lyophilized on a 28 L LSI tray lyophilizer, and lyophilized peptide was stored in a freezer at-10 to −25° C.

### 0.1 M NH HCO₃ pH 9 Mobile Phase System

The conditions for Stage 6 Salt Exchange (0.1 M NH₄HCO₃ PH 9 Mobile Phase System) were as follows:
Mobile Phase A (MPA): 0.1 M NH₄HCO₃ pH 9 in purified water
Mobile Phase B (MPB): 0.1 M NH₄HCO₃ pH 9 in 80% CH₃CN
Mobile Phase C (MPC): 0.1% TFA
Mobile Phase X (MPX): 80% CH₃CN Flow rate: 80 mL/min
Detector wavelength: 230 nm

Column equilibration was performed with 1.0 L of MPC. One half of main pool 5-1 (112.5 mL) from Stage 5 secondary purification was diluted 1:1 (v/v) with purified water and applied to the column, followed by two consecutive applications of 1.0 L of MPC. The product was eluted from the column with a gradient of 40→100% MPB in 45 minutes. After the product had eluted completely, the column was washed with 1.0 L of MPX. Individual fractions of 50 mL volume were collected and analyzed for purity by analytical UHPLC System 1 and System 2. Pooling of the selected main pool fractions into a main pool was performed according to the acceptance criteria for purity ≥98.0%, and pooling of the sidecut fractions into a sidecut pool was performed according to the acceptance criteria for purity ≥70%. Main pool 6-2 was stored in a refrigerator at ≤8° C. until further processing. Rear sidecut pool 6-2 was discarded. Subsequently, main pool 6-2 was lyophilized on a 28 L LSI tray lyophilizer, and lyophilized peptide was stored in a freezer at-10 to −25° C.

0.1 M NH₄HCO₃ pH 8 Mobile Phase System and Conversion of Hydrochloride Salt Form of CT-868 Lyophilizate to Ammonium Salt Form

The conditions for Stage 6 Salt Exchange (0.1 M NH₄HCO₃ pH 8 Mobile Phase System and Conversion of Hydrochloride Salt Form of CT-868 Lyophilizate to Ammonium Salt Form) were as follows:
Mobile Phase A (MPA): 0.1 M NH₄HCO₃ pH 8 in purified water Mobile Phase B (MPB): 0.1 M NH₄HCO₃ pH 8 in 80% CH₃CN
Mobile Phase C (MPC): 0.1% TFA Mobile Phase X (MPX): 80% CH₃CN Flow rate: 80 mL/min
Detector wavelength: 230 nm

Column equilibration was performed with 1.0 L of MPC. Lyophilized main pool (6-2, Stage 6) from CT-868 hydrochloride salt form Lot #1000071371 was dissolved in 300 mL of 0.1 M NH₄HCO₃ pH 8 at a concentration of 10 g/L and applied to the column, followed by two consecutive applications of 1.0 L of MPC. The product was eluted from the column with a gradient of 40+100% MPB in 45 minutes. After the product had eluted completely, the column was washed with 1.0 L of MPX. Individual fractions of 50 mL volume were collected and analyzed for purity by analytical UHPLC System 1 and System 2. Pooling of the selected main pool fractions into a main pool was performed according to the acceptance criteria for purity ≥98.0%, and pooling of the sidecut fractions into a sidecut pool was performed according to the acceptance criteria for purity ≥70%. Main pool 6-3 was stored in a refrigerator at ≤8° C. until further processing. Front and rear sidecut pools 6-3 were discarded.

The results are presented in Tables 11-14 below.

TABLE 11
Stage 6 Salt Exchange Purification UHPLC System 2 (C18) Analysis Results

| | | | Main Pool | Front | Rear | |
|---|---|---|---|---|---|---|
| | | Main Pool | Purity (%) | Sidecut | Sidecut | |
| Stage 6 Mobile Phase | Run | Fraction #/ | by System 2 | Fraction #/ | Fraction #/ | Fractions |
| System | # | Volume | (C18) | Volume | Volume | Discarded |
| 0.1M NH₄HCO₃ pH 8 | 6-1 | 4, 5/ | 99.4 | 1-3/ | None/ | None |
| | | 0.10 L | | 0.15 L | N/A | |
| 0.1M NH₄HCO₃ pH 9 | 6-2 | 1, 2/ | 99.6 | None/ | 3-5/ | None |
| | | 0.10 L | | N/A | 0.15 L | |
| 0.1M NH₄HCO₃ pH 8 and | 6-3 | 3, 5-8/ | 98.2 | 2.4/ | 9, 10/ | None |
| Hydrochloride Salt Form of | | 0.25 L | | 0.10 L | 0.10 L | |
| CT-868 Lyophilizate | | | | | | |

TABLE 12
Run 6-1 (0.1M NH₄HCO₃ pH 8) Fractions and Main
Pool Purity by System 1 (C8) and System 2 (C18)

| Fractions | C8 Purity (%) | C18 Purity (%) | Fraction volume (mL) |
|---|---|---|---|
| 1 | 96.7 | 98.5 | 50 |
| 2 | 97.5 | 97.6 | 50 |
| 3 | 94.4 | 96.1 | 50 |
| 4 | 96.9 | 98.9 | 50 |
| 5 | 97.3 | 98.5 | 50 |
| MP | 98.0 | 99.4 | 50 |

01 M NH₄HCO₃ pH 8
Came from pH=8.0
Main pool fractions were defined as ≥98.0% purity; sidecuts were defined as ≥70% purity
Buffers used:
A: 0.1 M NH₄HCO₃ (pH 8)
B: 0.1 M NH₄HCO₃ in 80% CH₃CN
C: 0.1% TFA
X: 80% CH₃CN
Gradient: 40-100% B in 45 minutes
Main pool: fractions 4 and 5 in C18

TABLE 13
Run 6-2 (0.1M NH₄HCO₃ pH 9) Fractions and Main
Pool Purity by System 1 (C8) and System 2 (C18)

| Fractions | C8 Purity (%) | C18 Purity (%) | Fraction volume (mL) |
|---|---|---|---|
| 1 | 98.1 | 99.9 | 50 |
| 2 | 95.9 | 99.1 | 50 |
| 3 | 90.1 | 95.1 | 50 |
| 4 | 66.4 | 72.2 | 50 |
| 5 | 97.0 | 99.6 | 50 |
| MP | 97.0 | 99.6 | |

Came from pH = 9.0; Main pool: C8 Fraction 1; C18 Fractions 1 and 2

TABLE 14
Run 6-3 (0.1 M NH4HCO3 pH 8 and Hydrochloride Salt
Form of CT-868 Lyophilizate) Fractions and Main
Pool Purity by System 1 (C8) and System 2 (C18)

| Fractions | C8 Purity (%) | C18 Purity (%) | Fraction volume (mL) |
|---|---|---|---|
| 1 | 41.9 | 7.0 | 50 |
| 2 | 89.7 | 91.7 | 50 |
| 3 | 97.1 | 99.2 | 50 |
| 4 | 94.9 | 96.1 | 50 |
| 5 | 96.6 | 98.9 | 50 |
| 6 | 95.6 | 98.4 | 50 |
| 7 | 96.0 | 98.5 | 50 |
| 8 | 96.6 | 98.5 | 50 |
| 9 | 84.9 | 91.2 | 50 |
| 10 | 71.5 | 74.2 | 50 |
| MP | 95.7 | 98.2 | |

HClto NH₄ salt
Main pool: C18 fractions 3 and 5-8

Following Stage 6 salt exchange purification, runs 6-1, 6-2, and 6-3 were lyophilized on a tray lyophilizer. The in-process tray lyophilization results and subsequent analytical UHPLC analysis are presented in Table 15 below.

Subsequently, main pool 6-3 was lyophilized on a 28 L LSI tray lyophilizer, and lyophilized peptide was stored in a freezer at-10 to −25° C.

TABLE 15
In-Process Tray Lyophilization - UHPLC System 2 (C18) Analysis Results

| | | Net Weight of | Purity (%) of In- | LSI |
|---|---|---|---|---|
| | | In- Process | Process Lyophilizate | (RRT/ |
| Stage 6 Mobile Phase System | Run | Lyophilizate (g) | by System 2 (C18) | %) |
| 0.1M NH₄HCO₃ pH 8 | 6-1 | 0.03 g¹ | 99.5 | 1.031/ |
| | | | | 0.2 |
| 0.1M NH₄HCO₃ pH 9 | 6-1 | 0.08 g¹ | 99.6 | 1.039/ |
| | | | | 0.1 |
| 0.1M NH₄HCO₃ pH 8 and | 6-3 | 0.15 g | 98.1 | 1.123/ |
| Hydrochloride Salt Form of | | | | 0.5 |
| CT-868 Lyophilizate | | | | |

¹The net weight of the in-process lyophilizate does not reflect the actual net weight.
Samples were removed during various stages of analysis and for the LC-MS study.
removed during various stages of analysis and for the LC-MS study.

### Solubility of CT-868 Peptide Ammonium Salt Form

Following in-process lyophilization, one sample of the lyophilized CT-868 ammonium salt form was dissolved in 0.1 M NH₄HCO₃ solution pH 9 at a concentration of 20 g/L, and one sample was dissolved in 0.1 M NH₄HCO₃ solution pH 10 at a concentration of 20 g/L. Dissolution of the samples was visually observed. The reconstituted CT-868 was lyophilized using a tray lyophilizer and LC-MS analysis was performed to determine peak purity.

Per visual observation of the dissolved samples, both buffers produced comparable dissolution results. The peptide dissolved at a concentration of 20 g/L but the recommendation is to apply a concentration of 15 g/L to decrease the possibility of precipitation.

### Stability of CT-868 Peptide Ammonium Salt Form

The CT-868 lyophilizate in the ammonium salt form from Stage 6 salt exchange purification was evaluated for purity at room temperature and drying at 35° C. over seven days. The lyophilizate samples were dissolved in 10 mM NH₄HCO₃ solution pH 8 at a concentration of 1 mg/mL, and analyzed by System 2 (C18).

The lyophilized run 6-3 main pool from Stage 6 salt exchange was examined for purity by System 2 (C18) at room temperature and drying at 35° C. over seven days (Table 16).

TABLE 16
CT-868 Ammonium Salt Form Stability at Room Temperature
and Drying at 35° C. Over Seven Days

| | t₀ | Day 1 | Day 2 | Day 3 | Day 6 | Day 7 |
|---|---|---|---|---|---|---|
| Purity at Room | 98.10% | 98.10% | 98.20% | 97.90% | 97.80% | 97.30% |
| Temperature (%) | | | | | | |
| Purity at 35° C. (%) | 98.10% | 98.10% | 98.10% | 98.10% | 98.10% | 97.60% |

The purity at the room temperature ranged between 98.2% (Day 2) to 97.3% (Day 7) for an overall range difference of 0.9%. The highest purity was observed after two days of storage at room temperature and the lowest purity was observed after 7 days of storage at room temperature. The starting (to) and last analysis (Day 7) are 0.8% apart in purity.

The purity for storage at 35° C. ranged between 98.1% (to) to 97.6% (Day 7) for an overall range difference of 0.5%. A consistent trend was observed for purity of 98.1% from the start of the analysis (to) through Day 6. The purity decreased from 98.1% to 97.6% on Day 7. The stability results show a slight purity decline for both room temperature and 35° C. conditions over seven days; however, the purity decline at room temperature decreases more gradually over time than at 35° C. In addition, fluctuations in the analytical method were also observed, therefore, the lower purities reported for Day 7 could also partially be related to the reproducibility of the method.

### Peak Purity of CT-868 Peptide Ammonium Salt Form by LC-MS

### Method

The LC-MS method information is presented below.
Test substance: CT-868
Formula: C₂₁₄H₃₂₃O₆₄N₄₇S₁
Molecular mass: mₘₒₙₒ=4607.310 u (monoisotopic)·
Instrument: Vanquish Horizon Binary UHPLC with 350 uL mixer.
Mass spectrometer: Thermo Q-Exactive Focus Hybrid Orbitrap MS
Eluent A: 10 mM ammonium bicarbonate
Eluent B: 90/10 (v/v) CH₃CN/H₂O
Diluent: 10 mM ammonium bicarbonate
Test substance solution: Approx. 2 mg/mL in diluent
Column: Waters Acquity UPLC BEHC18, 300 Å, 1.7 μm, 2.1×150 mm P/N 186003687
Detection: λ=220 nm
Evaluated range [m/z]: 200-2270
Injection volume: 3
Volume of eluent mixer: 350 L thermo static mixer
Threshold for Peak Purity: 0.5%

The lyophilized CT-868 ammonium salt form Runs 6-1 and 6-2 were reconstituted using the reconstitution solution of 0.1 M NH₄HCO₃ pH 9 and pH 10 at a concentration of 20 g/L. The solutions were lyophilized in a tray lyophilizer. LC-MS analysis was performed to determine peak purity. The results are presented below.

### Test Chromatograms

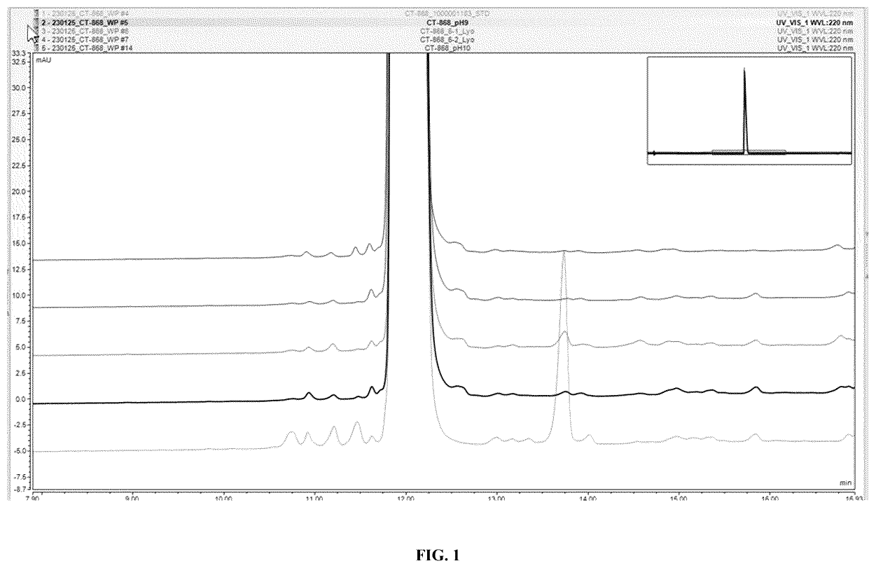

The overlay of samples is shown in FIG. 1. Samples were Standard, pH 9, pH 10, Lyophilizate 6-1, and Lyophilizate 6-2.

### Alignment of UV and MS Chromatogram

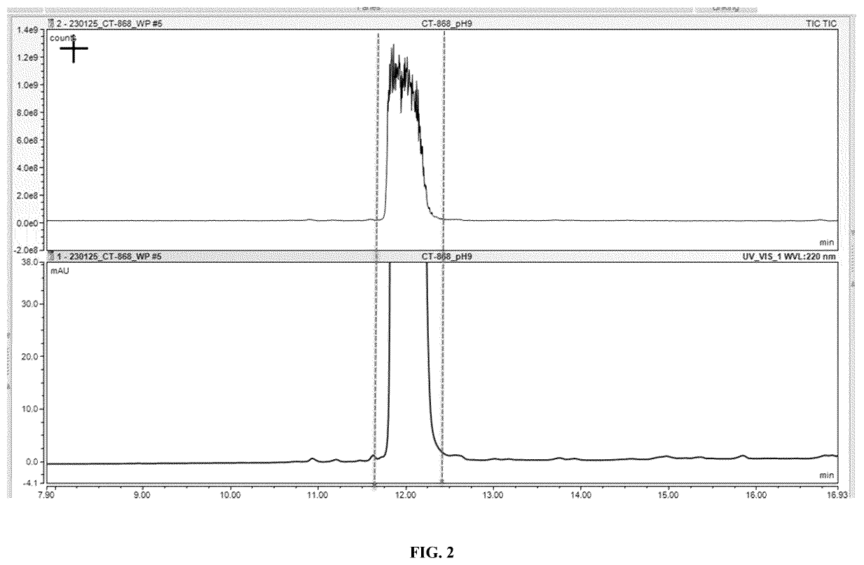

MS spectra and UV chromatograms of the samples were aligned based on the main peak and/or impurities in order to ensure the correct time range for the MS data. FIG. 2 illustrates this alignment.

### Signals in Main Peak Range

All observed m/z values in the range from 200 to 2270 with relative abundance (rel. a.)≥0.5% relative to the most abundant signal were evaluated. Next, for m/z values observed at or above reporting threshold, the extracted ion currents (EIC) were compared with the most abundant m/z signal presented for CT-868. Signals that were identified as product related, such as signals originating from different charge states or various salt adducts, were not investigated. For EICs of m/z values showing incongruent trends with the EIC of the product ion signal, the relative mass differences were determined. For impurity m/z signals that interfere with the m/z signal of a product-related artifact, the relative abundance given below represents the combined signal intensity of impurity and its corresponding artifact.

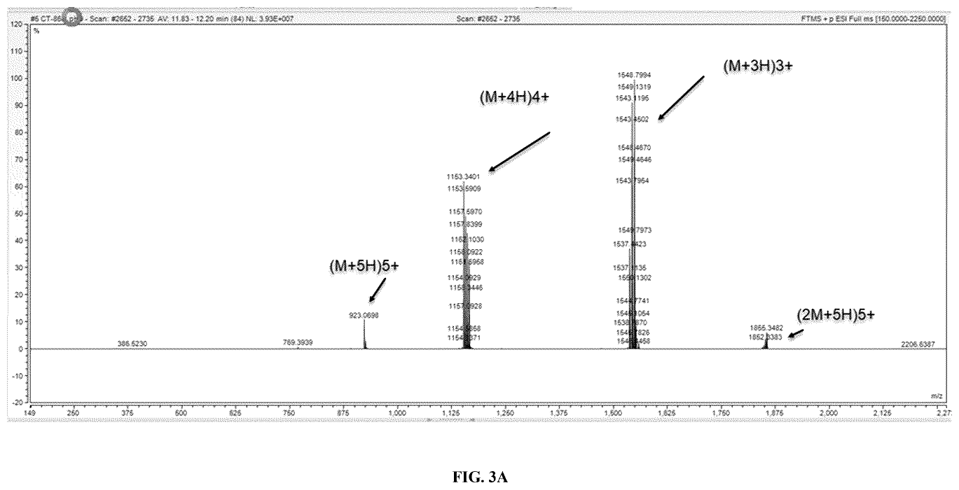

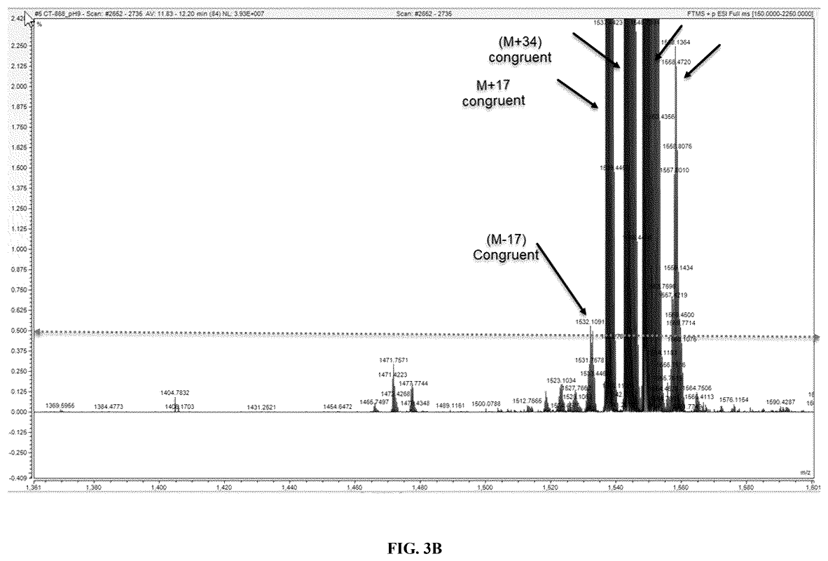

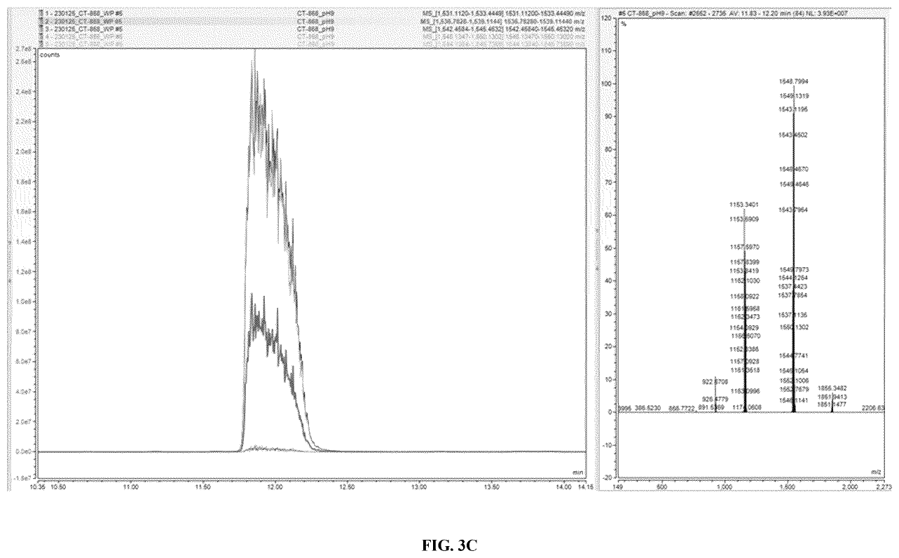

FIG. 3A shows Full scale mass spectra for CT-868 Lyophilizate_pH 9 sample. The Charge state of the target peak is shown. FIG. 3B shows the expanded scale mass spectra for freshly prepared CT-868_pH 9. The congruent mass signals are not reported below. FIG. 3C shows the MS spectrum of the main peak according to UV chromatogram of the CT-868 pH 9 sample.

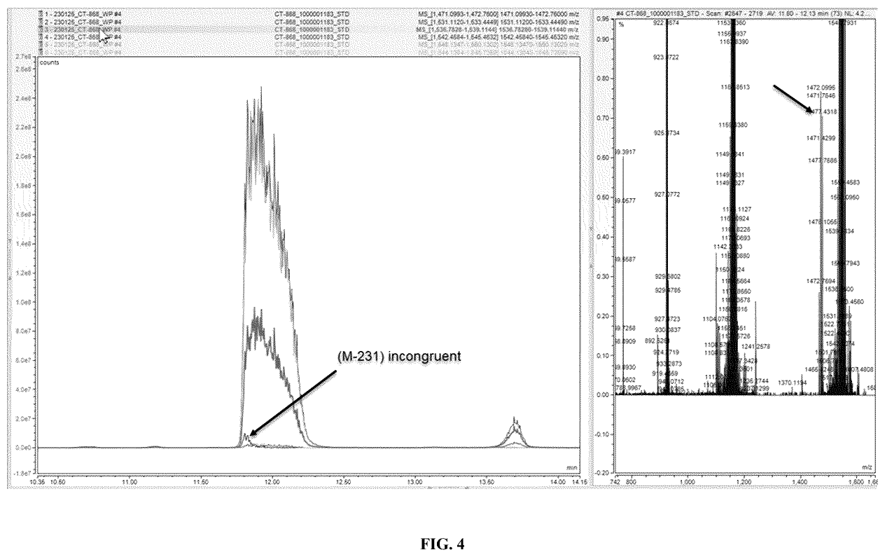

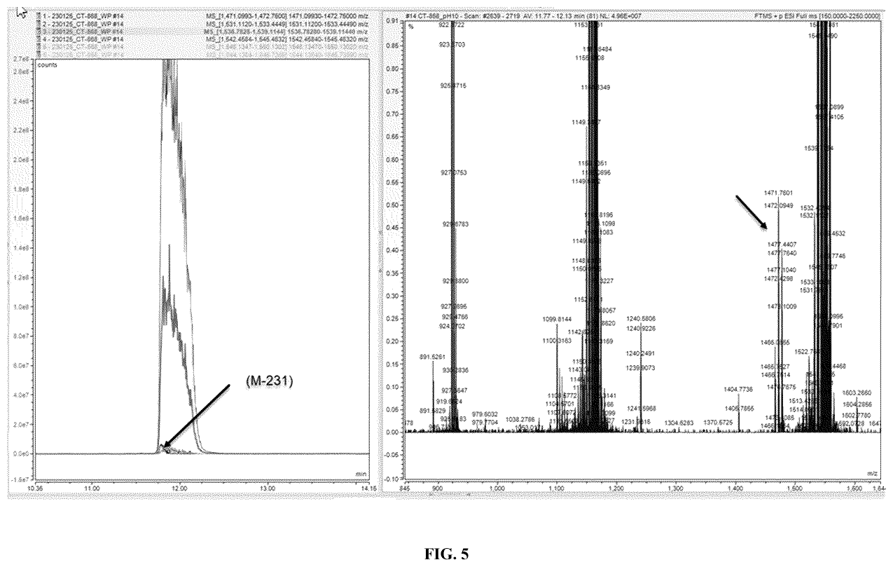

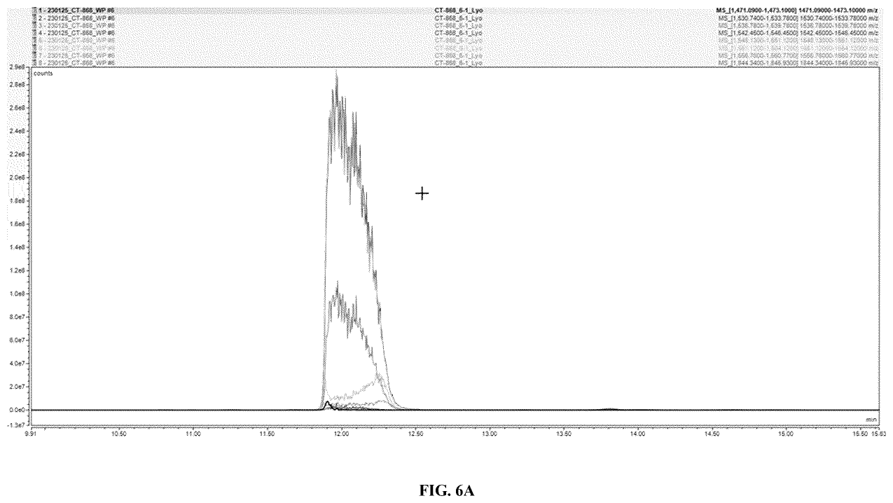

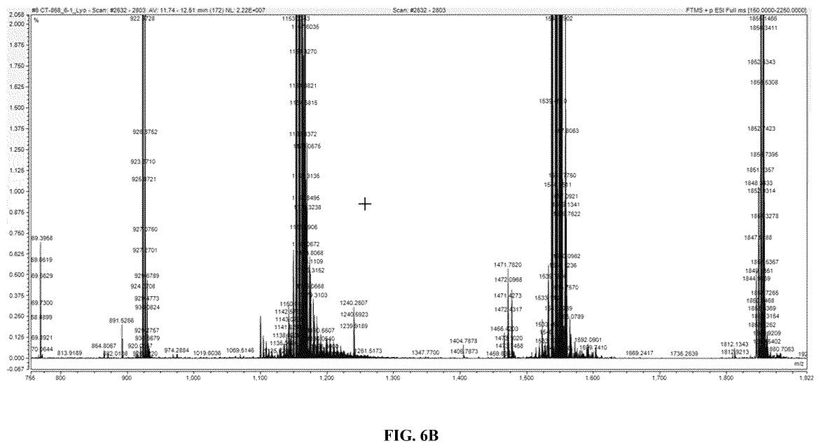

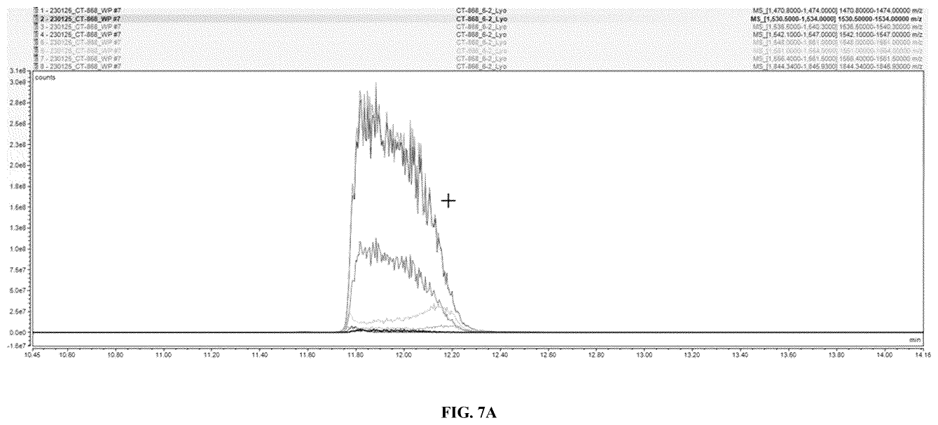

FIG. 4 shows MS spectrum of the main peak according to UV chromatogram of the CT-868 Standard. The impurity (M-231) Mass 4410.2307 is incongruent. FIG. 5 shows MS spectrum of the main peak according to UV chromatogram of CT-868_pH 10 sample. The impurity (M-231) Mass 4410.2307 is incongruent. FIGS. 6A and 6B show, respectively, the chromatogram and MS spectrum of the main peak according to UV chromatogram of CT-868_6-1_Lyo sample. FIGS. 7A and 7B show, respectively, the chromatogram and MS spectrum of the main peak according to UV chromatogram of CT-868_6-2_Lyo sample.

### Identified Impurities ≥0.5% (Monoisotopic Mass) Eluting in the Main Peak Range

One impurity ≥0.5% (monoisotopic mass) eluting in the main peak range was identified. Its characteristics are as follows:
Monoisotopic calculated: 4410.2307
Evaluated Ion: (M+2H+NH₄)3⁺
Δm [u] (M-231)
% rel. a. 0.5
Remarks: unknown identity (incongruent)
   Determination of Anions, Cations, and Acetate Content by Ion Chromatography (IC) at pH 9 and pH 10, and Residual Solvents by Head Space-Gas Chromatography (HS-GC) with Flame Ionization Detection (FID) at pH 9 and pH 10

Quantitative determination of TFA, chloride, phosphate, ammonium, TEA, and acetate at pH 9 and pH 10 was performed. The results are shown in Table 17 below.

TABLE 17
Results for Determination of Anions, Cations, and
Acetate Content by IC at pH 9 and pH 10, and Residual
Solvents by HS-GC with FID at pH 9 and pH 10

| Test Component | Level_pH 9 | Level_pH 10 |
|---|---|---|
| TFA | 0.18% | 0.17% |
| Chloride | 0.10% | 0.16% |
| Phosphate | 0.06% | 0.06% |
| Ammonium | 0.31% | 0.32% |
| TEA | 9.43 ppm | 9.26 ppm |
| Acetate | 0% | 0% |

### DISCUSSION

### In-Process Control Analytical UHPLC Method Comparison Results

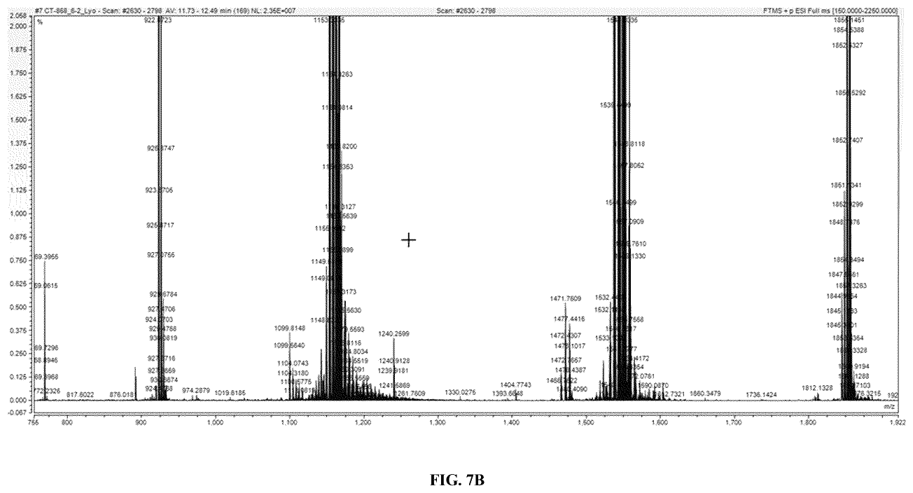

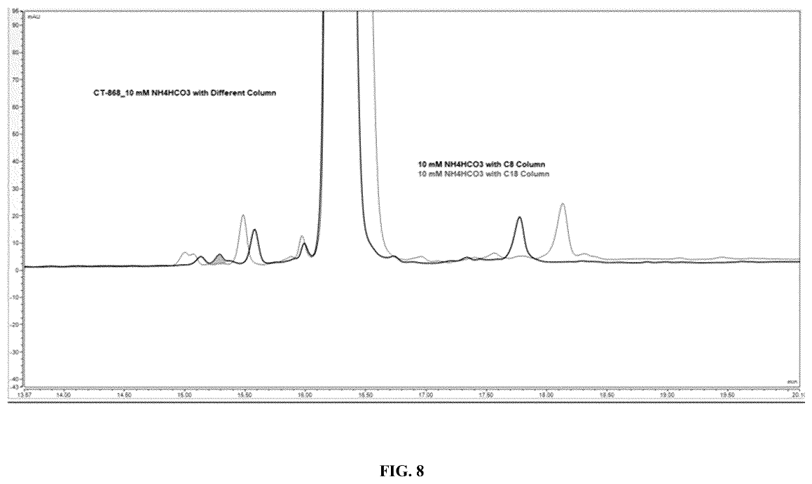

Overlay chromatogram of a representative Stage 4 primary purification run (Run 4-1) analyzed by analytical UHPLC System 1 (C8) and System 2 (C18) is presented in FIG. 8. The C18 column is recommended for analytical UHPLC analysis of CT-868. The observation is that C18 has better performance of the two columns due to sharper column resolution of the impurity peaks. The separation of baselines is not clearly defined, resulting in low selectivity.

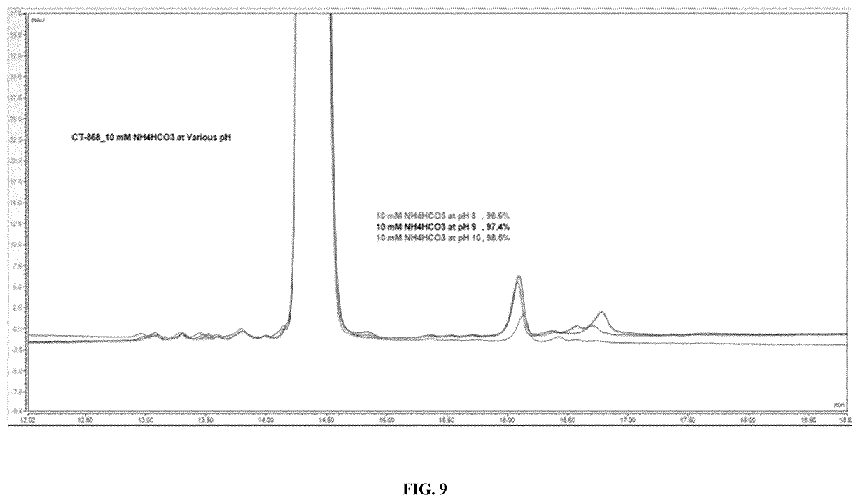

Solutions of standard sample and 10 mM NH₄HCO₃ at pH 8, pH 9, and pH 10 were analyzed by analytical UHPLC System 2 (C18). FIG. 9 shows the overlay chromatogram of the three UHPLC analysis. It was observed that 10 mM NH₄HCO₃ pH 8 had purity of 96.6%, 10 mM NH₄HCO₃ pH 9 had purity of 97.4%, and 10 mM NH₄HCO₃ pH 10 had purity of 98.5%. The observation is that although 10 mM NH₄HCO₃ pH 8 provides the lowest purity of CT-868 peptide, it resulted in a sharper resolution of the impurity peaks. As such, 10 mM NH₄HCO₃ at pH 8 is recommended as the analytical UHPLC mobile phase A.

### Evaluation of Purification Strategy by Preparative HPLC

Stage 4 primary purification with ammonium phosphate mobile phase system produced a failed run. High pressure stopped at 75% MPB during gradient run (scheduled gradient run: 40→80% MPB in 80 minutes). Product never came out throughout the gradient run. Fractions were collected when the column was washed with MPX.

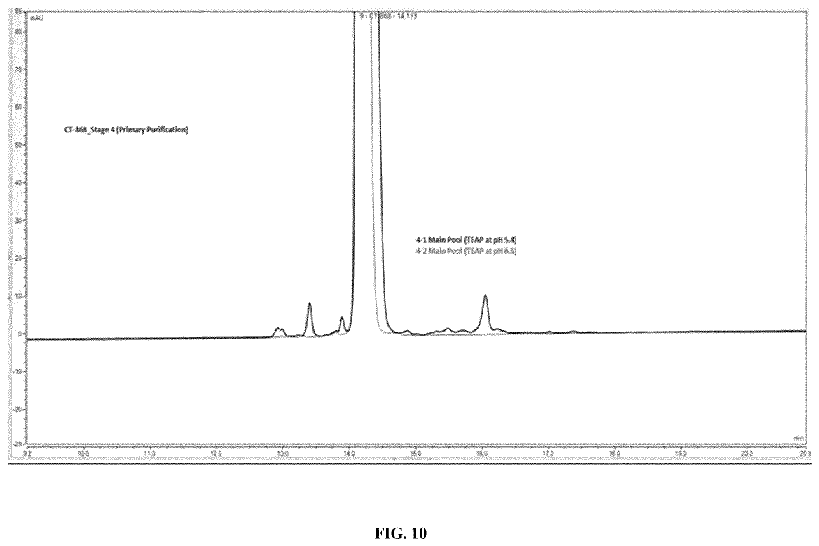

An overlay of Stage 4 primary purification Run 4-1 (TEAP pH 5.4 mobile phase system) and Run 4-2 (TEAP pH 6.5 mobile phase system) analyzed by analytical UHPLC System 2 (C18) is presented in FIG. 10. The observation is that TEAP pH 5.4 mobile phase system produces better resolution of impurity peaks in the overlay chromatogram. Although TEAP pH 6.5 mobile phase system yields more main pools with higher purity, due to the better resolution of impurity peaks TEAP pH 5.4 mobile phase system is recommended for Stage 4 primary purification of CT-868 peptide.

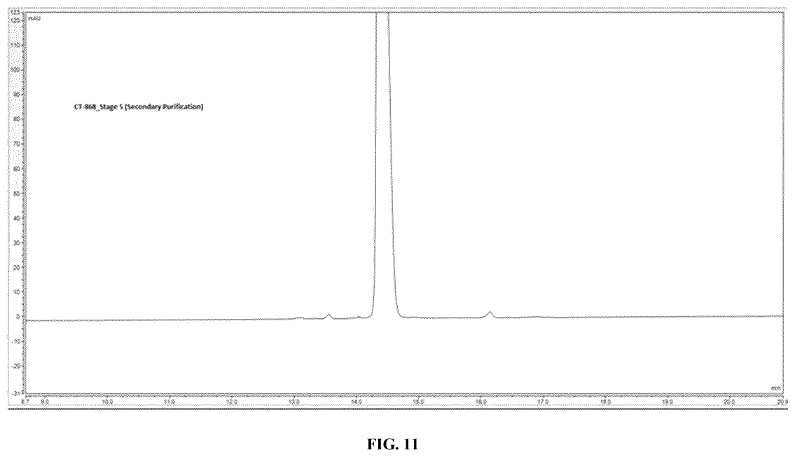

For the secondary purification (stage 5), run 5-1 in 0.1% TFA mobile phase system yielded three main pool fractions with the main pool purity of 98.4%. FIG. 11 presents analytical UHPLC analysis of Run 5-1 by System 2 (C18).

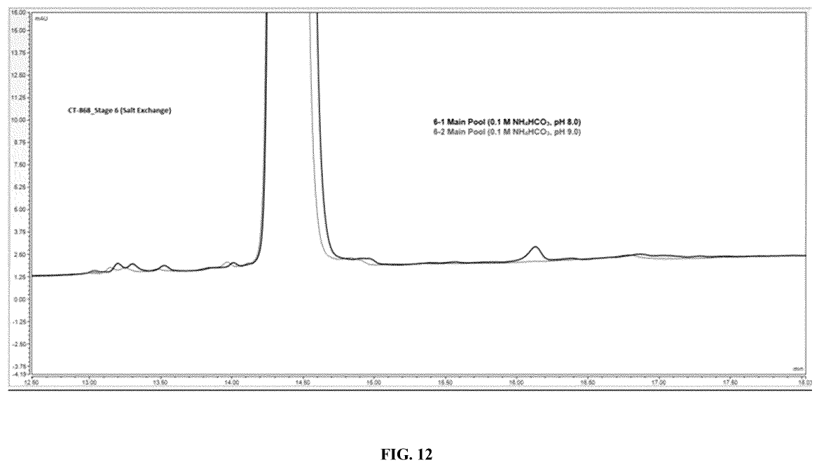

For salt exchange purification (stage 6), Run 6-1 in 0.1 M NH₄HCO₃ pH 8 mobile phase system yielded two main pool fractions with the main pool purity of 99.4%. Run 6-2 in 0.1 M NH₄HCO₃ pH 9 mobile phase system yielded two main pool fractions with the main pool purity of 99.6%. The main pool results from Run 6-1 and Run 6-2 are comparable. Overlay of Stage 6 purification Run 6-1 (0.1 M NH₄HCO₃ pH 8 mobile phase system) and Run 6-2 (0.1 M NH₄HCO₃ pH 9 mobile phase system) analyzed by analytical UHPLC System 2 (C₁₈) is presented in FIG. 12. The observation is that 0.1 M NH₄HCO₃ pH 8 mobile phase system produces better separation of impurity peaks at the beginning of gradient. Based on the above-mentioned results, 0.1 M NH₄HCO₃ pH 8 mobile phase system is recommended for Stage 6 salt exchange purification of CT-868 peptide.

### Evaluation of Solubility of CT-868 Peptide Ammonium Salt Form

Visual observation of the lyophilized CT-868 ammonium salt form dissolved at 20 mg/mL (weight of free peptide (i.e., without ammonium counterions)/volume) in 0.1 M NH₄HCO₃ solution pH 9 and in 0.1 M NH₄HCO₃ solution pH 10 noted comparable dissolution results. The peptide dissolved at a concentration of 20 g/L but the recommendation is to apply a concentration of 15 g/L to decrease the possibility of precipitation.

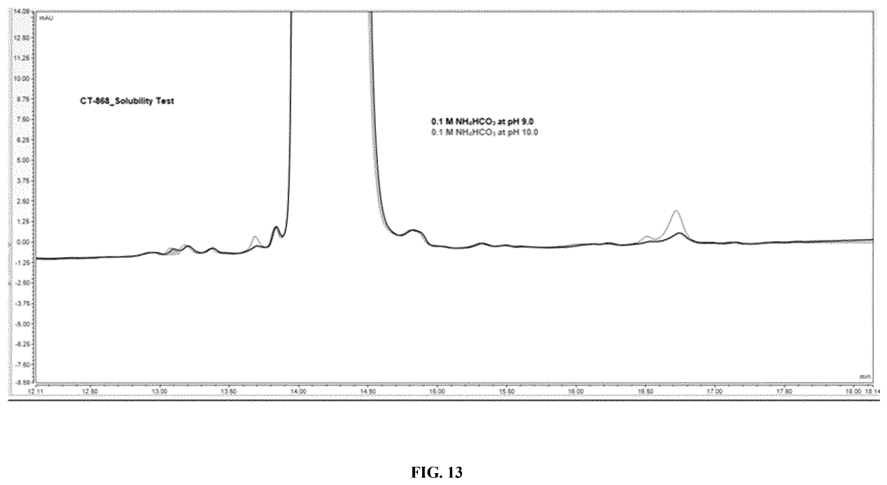

Overlay of analytical UHPLC analysis by System 2 (C18) of the dissolved CT-868 peptide in 0.1 M NH₄HCO₃ solution at pH 9 and pH 10 is presented in FIG. 13. The solution at pH 10 exhibited higher impurity profiles. Small scale results may not be similar for larger scale projects. Based on the above-mentioned results, 0.1 M NH₄HCO₃ pH 10 is recommended for dissolution of lyophilized CT-868 ammonium salt form prior to reconstitution.

### Evaluation of Stability of CT-868 Peptide Ammonium Salt Form

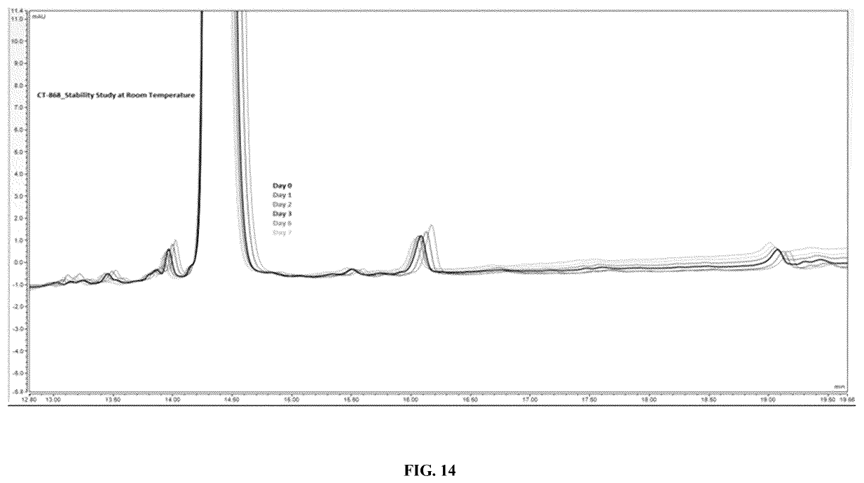

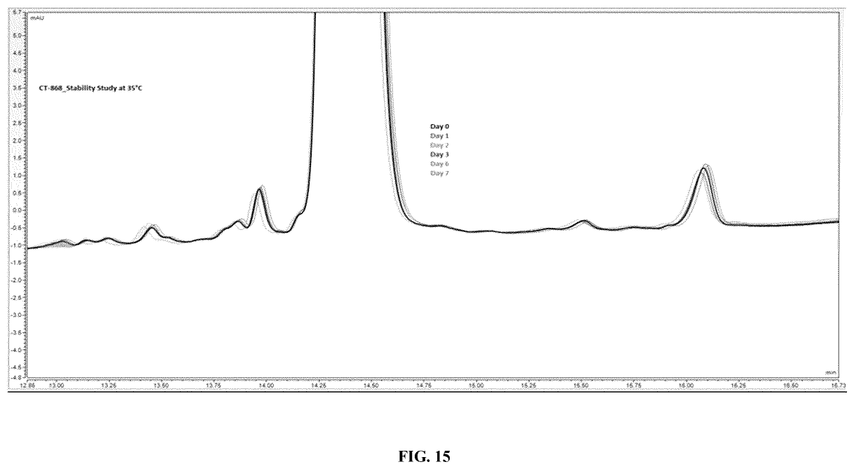

Overlay of analytical UHPLC analysis by System 2 (C18) of the in-process lyophilized CT-868 ammonium salt from Run 6-3 samples stored at room temperature over seven days is presented in FIG. 14. An overlay of analytical UHPLC analysis by System 2 (C18) of lyophilized CT-868 ammonium salt the Run 6-3 samples dried at 35° C. over seven days is presented in FIG. 15. Compared to product stored at 35° C., purity of product stored at room temperature decreased gradually over a seven-day period.

### CONCLUSION

The discussed experiments designed to evaluate selected parameters of the purification and salt exchange process of CT-868, and the conversion of CT-868 hydrochloride salt to CT-868 ammonium salt were successfully carried out.

With regard to the in-process control analytical UHPLC method, the C18 column is recommended for analytical UHPLC analysis of CT-868, and 10 mM NH₄HCO₃ at pH 8 is recommended as the analytical UHPLC mobile phase A.

With regard to the Preparative HPLC Purification Strategy, TEAP pH 5.4 mobile phase system is recommended for Stage 4 primary purification, 0.1% TFA mobile phase system is recommended for Stage 5 secondary purification, and 0.1 M NH₄HCO₃ pH 8 mobile phase system is recommended for Stage 6 salt exchange purification. 0.1 M NH₄HCO₃ pH 10 is recommended for dissolution of lyophilized CT-868 ammonium salt prior to reconstitution. The peptide dissolved at a reconstitution solution concentration of 20 g/L but the recommendation is to apply a concentration of 15 g/L to decrease the possibility of precipitation. An additional solution hold time study is required to ensure the stability of the product solution over the typical manufacturing time (<24 h) at the comparably high pH.

The stability results show a slight purity decline for both room temperature and 35° C. conditions over seven days; however, the purity decline at room temperature decreases more gradually over time than at 35° C. In addition, fluctuations in the analytical method were also observed, therefore, the lower purities reported for Day 7 could also partially be related to the reproducibility of the method.

### Example 3: Solid Phase Synthesis (SPPS) of CT-868

CT-868 is an N-terminal conjugated peptidyl compound with the structural formula (VI):

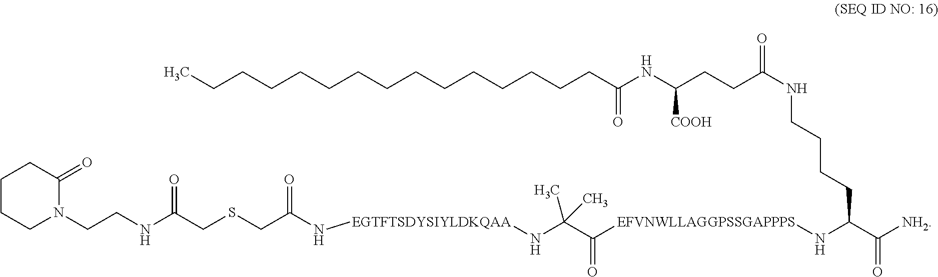

CT-868 was synthesized using solid phase peptide synthesis as described below.

### Step 1: Fmoc-Deprotection Cycle

The first step involved deprotection of the Fmoc-protecting group on the amino group of the Ramage amide AM resin (0.75-0.89 mmol/g resin). After the first coupling cycle, this step was performed to deprotect the previously coupled Fmoc-protected AA or dipeptide.

The experimental procedure for Fmoc deprotection was as follows: the resin was added to the reactor and swelled 2×15 min with DMF (10 mL/g initial resin) and then treated with 20% piperidine in DMF for 30 min as Fmoc-deprotection time. The resin was then washed once with DMF, once with isopropanol (IPA) and at least two times with DMF (10 mL/g initial resin [original resin dry weight before beginning of synthesis]) until the piperidine content of the washing solution was ≤2000 mg/kg (pH test).

In later repetitions of this step, the swelling was omitted. Table 18 shows a second Fmoc-deprotection of additional time at cycles 15-17.

TABLE 18
SPPS Conditions

| | | | Minimum | Fmoc- | |
|---|---|---|---|---|---|
| | Position | | coupling | deprotection | |
| | in | | duration | time | |
| Cycle | Sequence | Building Block | (hours:minutes) | (minutes) | Capping |
| 0 | N/A | Fmoc-Ramage AM-resin | n/a | 30 | No |
| 1 | 38 | Fmoc-Lys(γ-Glu- | 1:30 | 30 | No |
| | | palmitoyl)-OH | | | |
| 2 | 37 | Fmoc-Ser(tBu)-OH | 1:30 | 30 | No |
| 3 | 35-36 | Fmoc-Pro-Pro-OH | 1:30 | 30 | No |
| 4 | 33-34 | Fmoc-Ala-Pro-OH | 1:30 | 30 | No |
| 5 | 32 | Fmoc-Gly-OH | 1:30 | 30 | No |
| 6 | 31 | Fmoc-Ser(tBu)-OH | 1:30 | 30 | No |
| 7 | 30 | Fmoc-Ser(tBu)-OH | 1:30 | 30 | No |
| 8 | 28-29 | Fmoc-Gly-Pro-OH | 1:30 | 30 | No |
| 9 | 26-27 | Fmoc-Ala-Gly-OH | 1:30 | 30 | No |
| 10 | 25 | Fmoc-Leu-OH | 1:30 | 30 | No |
| 11 | 24 | Fmoc-Leu-OH | 1:30 | 30 | No |
| 12 | 23 | Fmoc-Trp(Boc)-OH | 1:30 | 30 | No |
| 13 | 22 | Fmoc-Asn(Trt)-OH | 1:30 | 30 | No |
| 14 | 21 | Fmoc-Val-OH | 6:00 | 30 | Yes |
| 15 | 20 | Fmoc-Phe-OH | 1:30 | 30 + 30 | No |
| 16 | 19 | Fmoc-Glu(OtBu)-OH × H₂O | 3:00 | 30 + 30 | Yes |
| 17 | 18 | Fmoc-Aib-OH | 1:30 | 30 + 30 | No |
| 18 | 17 | Fmoc-Ala-OH × H₂O | 1:30 | Np* | No |
| 18 | 17 | Fmoc-Ala-OH × H₂O | 1:30 | 30 | No |
| 19 | 16 | Fmoc-Ala-OH × H₂O | 1:30 | 30 | No |
| 20 | 15 | Fmoc-Gln(Trt)-OH | 1:30 | 30 | No |
| 21 | 14 | Fmoc-Lys(Boc)-OH | 1:30 | 30 | No |
| 22 | 13 | Fmoc-Asp(OMpe)-OH | 1:30 | 30 | No |
| 23 | 12 | Fmoc-Leu-OH | 3:00 | 30 | Yes |
| 24 | 11 | Fmoc-Tyr(tBu)-OH | 3:00 | 30 | Yes |
| 25 | 10 | Fmoc-Ile-OH | 3:00 | 30 | Yes |
| 26 | 9 | Fmoc-Ser(tBu)-OH | 3:00 | 30 | Yes |
| 27 | 8 | Fmoc-Tyr(tBu)-OH | 1:30 | 30 | No |
| 28 | 7 | Fmoc-Asp(OMpe)-OH | 3:00 | 30 | Yes |
| 29 | 5-6 | Fmoc-Thr(tBu)-Ser(ΨMe, Mepro)-OH | 3:00 | 30 | No |
| 30 | 4 | Fmoc-Phe-OH | 3:00 | 30 | No |
| 31 | 2-3 | Fmoc-Gly-Thr(ΨMe, Mepro)-OH | 3:00 | 30 | Yes |
| 32 | 1 | Fmoc-Glu(OtBu)-OH × H₂O | 1:30 | 30 | No |
| 33 | N-terminal | 2-(2-Piperidon-1-yl)-ethylcarbamoylmethyl- | 4:00 | n/a | No |
| | modification | thioacetic acid | | | |

*two times addition of activated Fmoc-Ala-OH

### Step 2: Coupling Cycle

The second step involved a solid phase peptide synthesis (SPPS) wherein the carbonyl group/C-terminus of the amino acid (AA) at position 38, i.e., lysine (Lys (y-Glu-palmitoyl)), with its N-terminus protected by an Fmoc group, was covalently bound to the deprotected amino group of the linking group of the Ramage amide AM resin. This step involved treating the resin and Fmoc-Lys (palmitoyl-Glu-OtBu)-OH with N,N-diisopropylcarbodiimide (DIC) and Oxyma Pure (CAS-No. 3849-21-6) in DMF for at least 1.5 hours at ambient temperature (AT), yielding Fmoc-Lys (palmitoyl-Glu-OtBu)-resin.

The experimental procedure for solid phase peptide synthesis was as follows: Fmoc-Lys (palmitoyl-Glu-OtBu)-OH (1.5 eq) and Oxyma Pure (2.3 eq) were dissolved in DMF (10 mL/g initial resin) to form a clear solution. Prior to coupling of the amino acid, DIC (2.9 eq) was added, and the solution was stirred for 15 minutes as pre-activation time before adding the preactivated amino acid solution to the resin and stirring for at least 1.5 hours at ambient temperature. After the coupling was completed, the amino acid solution was removed by filtration.

After certain coupling steps (as described in Table 18), a capping solution of acetic acid anhydride and pyridine in DMF (ratio: Jan. 1, 1950, v/v/v, 10 mL/g initial resin) was added to the resin and stirred for 10 minutes at ambient temperature. The capping solution was then removed by filtration and the resin was washed with DMF.

In later repetitions of this step, the minimum coupling times were varied between 1.5-6 hours as shown in Table 18. A capping step was performed for the coupling cycles noted in Table 18. The other parameters were adapted to 2.0 eq AA building block, 3.9 eq DIC, and 3.1 eq Oxyma Pure. The final coupling cycle 33 was again performed with 1.5 eq AA building block, 2.9 eq DIC, and 2.3 eq Oxyma Pure.

### Final Washing and Drying Step

Following the final coupling step of 2-(2-Piperidon-1-yl)-ethylcarbamoylmethylthioacetic acid, the resin was washed alternatingly with DMF and IPA three times and then three further times with IPA. The resin was then removed from the reactor and dried under reduced pressure at ambient temperature.

Cleavage from Resin and Acidic Deprotection

Cleavage of the resin-bound protected peptide precursor of CT-868 from the resin and simultaneous deprotection of the sidechain protecting groups of the amino acids was accomplished by treatment of the peptide resin with trifluoroacetic acid (TFA) in the presence of suitable scavengers. After filtering off and washing the resin, the product was precipitated in a suitable antisolvent. The obtained CT-868 (TFA salt, crude) was washed with antisolvent and dried under reduced pressure. The details of this procedure are described in Table 19.

TABLE 19
Conditions for Cleavage from Resin and Acidic Deprotection

| Step | Protocol Description |
|---|---|
| Cocktail | A cleavage cocktail (8 L/kg resin) was made consisting of TFA, ethane-1,2- |
| | dithiol (EDT), triisopropylsilane (TIS) and water (90.0:5.0:2.5:2.5, v/v/v/v) |
| | and held at ≤ −10° C. in a cleavage reactor. |
| Cleavage | The peptide resin was added to the cleavage cocktail and the solution was then |
| | stirred for 2 hours, maintaining a temperature of 21 ± 3° C. |
| Post-Cleavage | NH₄I and Vitamin C was added, and the solution was stirred for a further 10 |
| | min. |
| Filtration | The product solution was filtrated into a precipitation reactor and cooled |
| | to −15 ± 5° C. |
| Precipitation | Diisopropyl ether (IPE, 12 L/kg resin) was cooled to −20° C. and slowly added |
| | (75 min) to the cleavage solution in the precipitation reactor, maintaining an |
| | internal temperature of -15 + 5° C. |
| | A second portion of diisopropyl ether (IPE, 18 L/kg resin) was cooled to −20° |
| | C. and slowly added (69 min) to the cleavage solution in the precipitation |
| | reactor, maintaining an internal temperature of −15 ± 5° C. |
| Ageing | After addition of the IPE as antisolvent, the suspension was stirred at −2° C. for |
| | 30 min. |
| Isolation | The product was isolated by filtration and washed with IPE three times before |
| | drying under reduced pressure at ambient temperature for 72 h. |

### Purification by Preparative HPLC

Purification of the crude material of CT-868 (TFA salt) was achieved using a one-dimensional purification strategy by reversed phase preparative HPLC. The peptide was dissolved and subjected to decarboxylation and isoacyl rearrangement as described in Tables 20A and 20B. The purification was carried out using NH₄OAc as a modifier in aqueous, acetonitrile (MeCN)-containing eluents. The product was eluted with a gradient of increasing MeCN concentration. Suitably pure fractions were combined and concentrated in the subsequent ultrafiltration step, while less pure fractions could be recycled in further purification runs. The details of this procedure are described in Table 21.

TABLE 20A
Pre-Treatment of Feed Solution

| Step | Protocol Description |
|---|---|
| Product | The crude product is dissolved in 50% MeCN (v/v) in water at a |
| dissolution | concentration of 40 g/L. |
| pH adjustment | The pH of the solution is adjusted to 4.0 ± 0.5 using ammonium |
| | hydroxide solution (~25% NH₃ in water) |
| Decarboxylation | The solution is stirred at 40° C. for 30 ± 15 min |
| pH adjustment | The pH of the solution is adjusted to 7.0 ± 0.5 using ammonium |
| | hydroxide solution (~25% NH₃ in water) |

TABLE 20B
Purification Conditions

| Step | Protocol Description | |
|---|---|---|
| Stationary Phase | Reversed phase silicagel with C18 modification (particle size | |
| | 8 μm, pore size 100 Å) | |
| Full Load | 0.584-0.799 g/cm² g (gross) on column with 30 cm bed | |
| | height | |
| Eluents | A: 0.05M NH₄OAc, 3% MeCN | |
| | B: 0.05M NH₄OAc, 80% MeCN | |
| Gradient | 0.0 min | 0% B |
| | 1.0 min | 29% B |
| | 142 min | 100% B |
| Flow | 941 cm/h | |
| Detection | 280 nm | |
| Fractions | 30-60 s fractions Analyzed by U-HPLC | |

TABLE 21
Ultrafiltration Conditions

| Step | Protocol Description |
|---|---|
| Cassette | 3x Cellulose-based TFF membrane, cutoff: 2 |
| | kDa, 0.6 m² |
| Feed pressure | 2.50 bar |
| Retentate pressure | 2.40 bar |
| TMP | 2.45 bar |

### Tangential Flow Filtration

Concentration of the fraction pool from preparative HPLC was performed in portions via ultrafiltration. This was carried out in two steps (Concentration I and Final Concentration) with an intermediate diafiltration with five volumes of purified water. The concentrated product solution was filtrated into a retentate bag for storage until the final reconstruction step. The relevant parameters are described in Table 21.

### Final Reconstitution

The purified and concentrated material obtained from the tangential flow filtration step was homogenized. Ion exchange from ammonium to sodium was achieved by addition of aqueous 1.0 M Na₂CO₃. The solution was then microfiltered using a cellulose acetate membrane with a pore size of 0.2 μm, and the final product was isolated by lyophilization (Table 22).

TABLE 22
Lyophilization Conditions

| | Shelf | | | | |
|---|---|---|---|---|---|
| | Temperature | Hold/ | Rate | Time | Pressure |
| Cycle Phase | (° C.) | Rate | (° C./min) | (min) | (mbar) |
| Load | Ambient | Hold | | | Ambient |
| Freeze | Ambient | Rate | n.d.¹⁾ | n.d.¹⁾ | Ambient |
| Freeze | −50 | Hold | | 240 | Ambient |
| Freeze | −50 | Hold | | n.d.²⁾ | 1.0 |
| Primary | 0 | Rate | 0.21 | 240 | 1.0 |
| drying | | | | | |
| Primary | 22 | Rate | 0.06 | 360 | 1.0 |
| drying | | | | | |
| Secondary | 22 | Hold | | n.d.³⁾ | 1.0 |
| drying | | | | | |
| Unload | Ambient | Hold | | | Ambient |

¹⁾The lyophilization shelves are cooled until a temperature set point of -50° C. is reached.
²⁾Vacuum is applied until a vacuum set point of 1.0 mbar is reached.
³⁾The secondary drying conditions are applied until the product temperature remains constant (±0.2° C.) over a period of at least 2 h. Product temperature is monitored by temperature probes inserted into the product solution.

### Yield and Purity

The purity of the isolated product after lyophilization was determined by analytical HPLC. Analytical HPLC was performed on the Thermo Scientific™ Vanquish™ UHPLC system or Dionex Ultimate 3000 RS UHPLC system using the AQUITY Premier CSH C18 (1.7 μm, 2.1×150 mm) column, with a flow rate of 0.37 mL/min, column temperature of 50° C., and UV detection at 220 nm. Buffer A was 0.1% TFA in ACN/H₂O (30:70), v/v/volumes and buffer B was 0.1% TFA in ACN/H₂O (80:20), v/v/v. The gradient used for the analytical method is shown in Table 23.

TABLE 23
Gradient for HPLC Analysis

| Time (min) | % B |
|---|---|
| 0.0 | 30 |
| 1.0 | 30 |
| 26.0 | 80 |
| 26.1 | 100 |
| 28.0 | 100 |
| 28.1 | 30 |
| 35.0 | 30 |

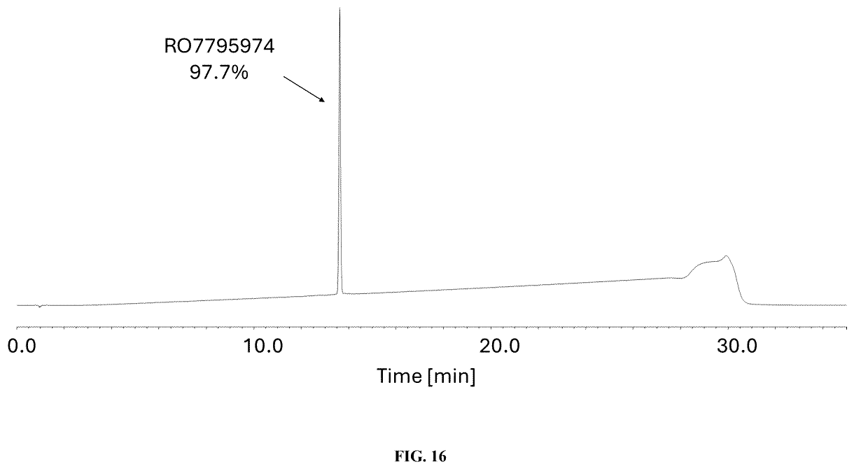

The overall yield of this manufacturing campaign, comprising the SPPS peptide formation, cleavage from the resin, and purification, was 45%. After lyophilization, the isolated CT-868 showed a purity of 97.7%, as shown by HPLC analysis (FIG. 16).

### Example 4: Synthesis of 2-((2-(2-oxopiperidin-1-yl)ethylcarbamoyl)methylthio)acetic acid (III)

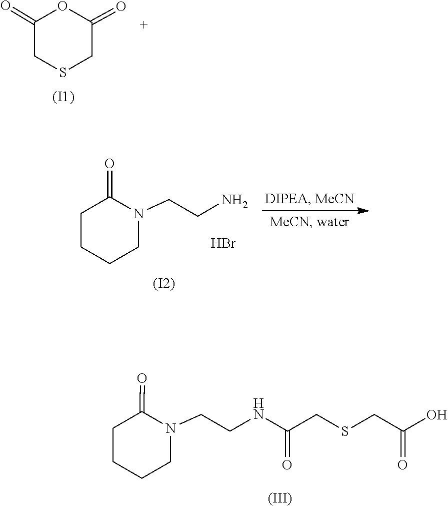

### Synthesis of 2-(2-Piperidon-1-yl)-ethylamine hydrobromide (I2)

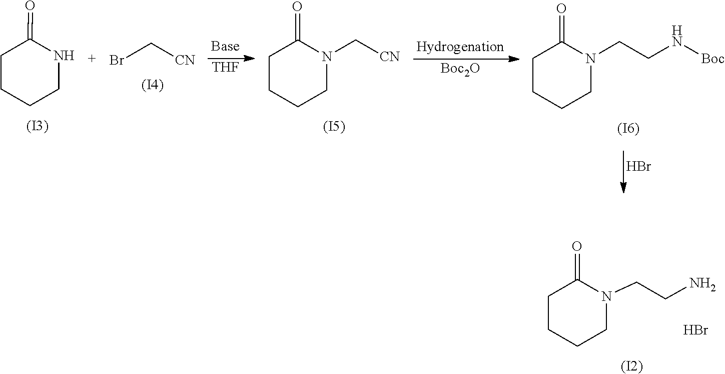

### Synthesis of 2-(2-Piperidon-1-yl)-acetonitrile of formula (I5)

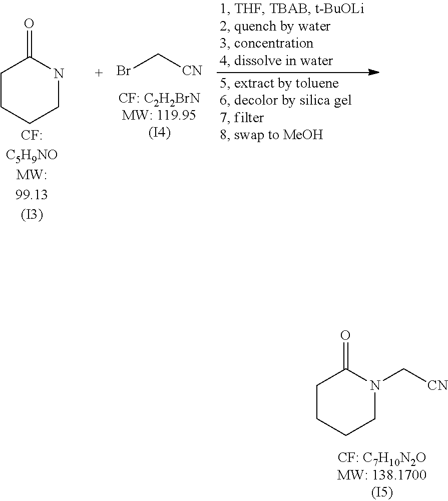

Initially, 2-piperidone (I3) (1.0 eq., 1.0 wt.) and THF (10.7 wt., 12 volumes) were charged into the reactor. The temperature was adjusted to between 1° and 25° C. Subsequently, in step 1, t-BuOLi (0.4 eq.) in THF solution was charged into the reactor, and the mixture was stirred for 30-60 minutes. TBAB (tetrabutylammonium bromide) (0.01 eq.) was then charged into the reactor. Bromo acetonitrile (I4) (0.4 eq.) in two volumes THF was then charged into the reactor over a period of 2-4 hours. The reaction mixture was agitated for an additional 30-60 minutes.

Following this, a second portion of t-BuOLi (0.4 eq.) in THE solution was charged into the reactor and stirred for 30-60 minutes. Bromo acetonitrile (0.4 eq.) in two volumes THF was then charged into the reactor over 2-4 hours. The mixture was agitated for 30-60 minutes.

Subsequently, a third portion of t-BuOLi (0.4 eq.) in THE solution was charged into the reactor and stirred for 30-60 minutes. Bromo acetonitrile (0.4 eq.) in two volumes THF was charged into the reactor over 2-4 hours. The mixture was agitated for 30-60 minutes. Water (0.22 wt.) was charged to the reactor at 10-25° C. The mixture was agitated for 20-60 minutes. The reaction mixture was then concentrated to 4-5 volumes below 45° C. Toluene (4.36 wt.) was charged to the reactor, and the mixture was again concentrated to 4-5 volumes below 45° C. The mixture was subsequently cooled to 20-30° C., and water (15.0 wt.) was charged into the reactor.

Toluene (17.44 wt.) was charged into the reactor. The mixture was agitated for 20-30 minutes, allowed to settle, and the two phases were separated. The product was then extracted by sequentially charging toluene to the reactor, with agitation and phase separation after each addition. The organic phases were then combined. The combined organic phase was washed with saturated NaCl aqueous solution (5.0 wt.), agitated for 20-30 minutes, allowed to settle, and the two phases were separated.

Silica gel (200-300 mesh) (0.5 wt.) was charged to the reactor and stirred for 1-3 hours. The mixture was then filtered, and the filter cake was rinsed with toluene (4.36 wt.), followed by a second rinse with toluene (4.36 wt.). The organic phases were combined.

The combined organic phase was concentrated to 0.8-1.2 volumes below 50° C. MeOH (3.95 wt.) was charged to the reactor, and the mixture was again concentrated to 0.8-1.2 volumes below 50° C. This was followed by charging MeOH (3.95 wt.) and concentrating to 0.8-1.2 volumes below 50° C. Finally, MeOH (0.79 wt.) was charged to the reactor, and the mixture was stirred for 10-30 minutes. The product was then discharged, weighed, and sampled for analysis, reporting a yield of 60.5% and a purity (HPLC, 94.2% a/a) for 2-(2-Piperidon-1-yl)-acetonitrile (I5).

The 2-(2-Piperidon-1-yl)-acetonitrile (I5) in MeOH solution was concentrated to 0.8-1.2 volumes below 60° C. THF (2.0 wt.) was charged to the reactor, and the mixture was concentrated to 0.8-1.2 volumes below 60° C. THF (2.0 wt.) was then charged, and the mixture was concentrated to 0.8-1.2 volumes below 60° C. Finally, THF (2.0 wt.) was charged into the reactor. An in-process control was performed, with the residual MeOH being ≤0.10% (GC, % w/w). The product was discharged, and the reactor was rinsed with THF (0.2 wt.) to obtain the 2-(2-Piperidon-1-yl)-acetonitrile (I5) THF solution.

### Synthesis of Boc-protected 2-(2-Piperidon-1-yl)-ethylamine (I6)

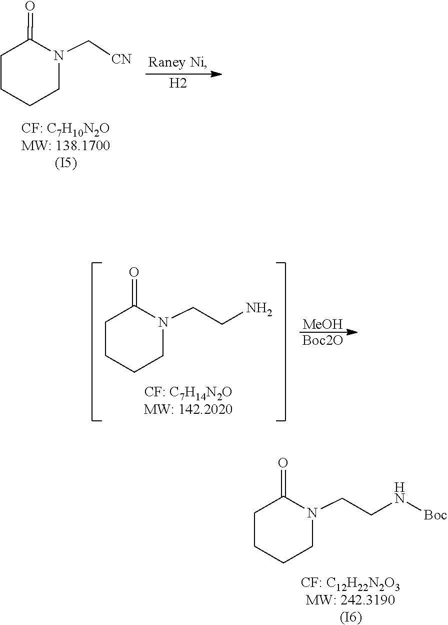

### Reaction 1: Reductive Amination

The autoclave reactor was purged with nitrogen three times to ensure an inert atmosphere. 2-(2-Piperidon-1-yl)-acetonitrile (I5) (1.0 eq., 1.0 wt.) in THE solution was charged into the reactor. Ammonia in THF (approximately 0.91 M, 27.0 wt.) was then charged into the reactor at a temperature of 0-5° C. Subsequently, Raney Ni (2.7 wt., with a KF of ≤0.2%) was charged into the autoclave at 0-5° C. Prior to charging, Raney Ni was rinsed with THF (40.8 wt.) until its water content (KF) was ≤0.2%. Ammonia was bubbled into the reactor at 2 bar pressure at 0-5° C. for 2-3 hours. In-process control confirmed the concentration of ammonia (≥1.5% w/w). Hydrogen was then fed to the reactor, and the pressure was maintained at 7-9 bar. The temperature was adjusted to 20-30° C. The reaction mixture was agitated for 12-24 hours.

The reaction mixture was filtered through a celite pad (0.1 wt.) at 20-30° C. The filter cake was then washed with THF (1.6 wt.) at 20-30° C.

The organic phase was concentrated under vacuum. The concentrated organic phase was transferred with MeOH (1.0 wt.). MeOH (4.0 wt.) was added into the reactor, and the organic phase was concentrated to 1.5 volumes under vacuum. This step was repeated twice more with MeOH (4.0 wt.) and MeOH (4.0 wt.), each time concentrating the organic phase to 1.5 volumes under vacuum. Finally, MeOH (6.4 wt.) was added into the reactor. The resulting solution was discharged, weighed, and sampled for analysis to report the purity of the intermediate 2-(2-Piperidon-1-yl)-ethylamine (HPLC, 93.9% a/a).

### Reaction 2: Boc-Protection

2-(2-Piperidon-1-yl)-ethylamine in MeOH solution (1.0 eq.) was charged into the reactor. Boc₂O (1.58 wt., dissolved in 1.6 wt. MeOH) was charged to the reactor at 20-30° C. The reaction mixture was agitated at 20-30° C. for 1-4 hours.

The reaction solution was concentrated to 1-1.5 volumes under vacuum at a temperature below 45° C. Water 1 (5.0 wt.) was charged to the reactor at 20-25° C. EtOAc (6.3 wt.) was then charged, and the mixture was agitated for 10-30 minutes.

The solution was filtered through a celite pad (0.2 wt.). The filter cake was then washed with EtOAc (2.7 wt.) at 20-25° C.

The filtrate was then separated. EtOAc (6.3 wt.) was charged to the water layer, agitated for 10-30 minutes, and the two layers were separated. This extraction step was repeated once more with EtOAc (6.3 wt.). The organic layers were then combined. Activated carbon (0.2 wt./wt.) was charged to the combined organic layers, and the mixture was stirred at 20-25° C. for 1-2 hours.

The solution was filtered through celite (0.2 wt.). The filter cake was washed with EtOAc (1.8 wt.) at 20-25° C.

The filtrate was concentrated to 4-5 volumes under vacuum at a temperature below 45° C. The product was then discharged, and a sample was taken for analysis. The Boc-protected 2-(2-Piperidon-1-yl)-ethylamine (I6) was obtained in an overall yield of 90% and a purity of 94.1% (HPLC).

### Synthesis of 2-(2-Piperidon-1-yl)-ethylamine hydrobromide (I2)

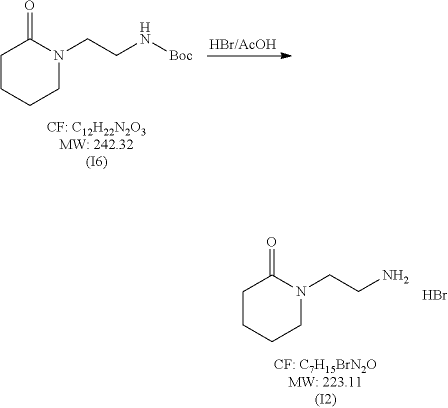 [SMILES: CC(C)(C)OC(=O)NCCN1CCCCC1=O ; NCCN1CCCCC1=O ; Br]

Boc-protected 2-(2-Piperidon-1-yl)-ethylamine (I6) (1.0 eq., 1.0 w/w) in EtOAc solution and EtOAc (13.5 wt.) were charged into the reactor at 20-30° C. The temperature was maintained at 20-30° C., and 33% HBr in HOAc (3.26 w/w) was charged dropwise to the reactor. The reaction mixture was agitated for 4-6 hours at 20-30° C.

The reaction mixture was filtered, and the filter cake was rinsed with EtOAc (3.6 wt.).

The wet cake was charged into the reactor at 20-30° C. MeOH (2.37 w/w) was charged into the reactor at 20-30° C. EtOAc (5.4 wt.) was then charged dropwise to the reactor at 20-30° C. The temperature was maintained at 20-30° C., and the reaction mixture was agitated for 6-18 hours.

The mixture was filtered, and the filter cake was rinsed twice with EtOAc (3.6 wt.).

The wet cake was dried at 35-45° C. for 12-18 hours, which afforded 2-(2-Piperidon-1-yl)-ethylamine hydrobromide (I2) in a yield of 90% with a purity of 99.9%.

### Synthesis of 2-((2-(2-oxopiperidin-1-yl)ethylcarbamoyl)methylthio) acetic acid (III))

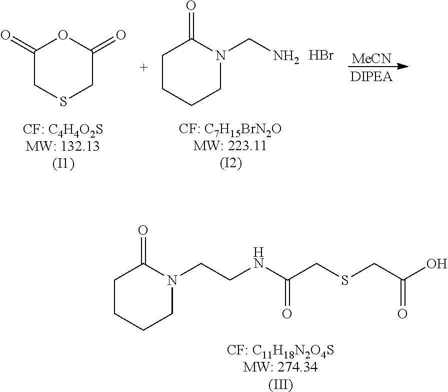

### Reaction Procedure

2-(2-Piperidon-1-yl)-ethylamine hydrobromide (I2)(1.0 eq., 1.0 wt.) was charged into the reactor at 10-20° C. Acetonitrile (MeCN) 1 (31.6 wt., 45 volumes) was subsequently charged into the reactor at 10-20° C. DIPEA (1.56 eq., 0.9 wt.) was introduced into the reactor at 10-20° C., and the associated pipe was rinsed with MeCN 2 (0.5 volume, 0.40 wt.). Thiodiglycolic anhydride (I1) (1.03 eq., 0.61 wt.) in MeCN (3.95 wt.) was charged into the reactor at 10-20° C., and its pipe was rinsed with MeCN (0.5 volume, 0.40 wt.). The reactor contents were agitated at 15-25° C. for 1-2 hours.

The organic phase was concentrated to 6-8 volumes below 45° C. under vacuum. The reaction mixture was agitated at 15-25° C. for 4-6 hours.

The mixture was centrifuged, and the filter cake was rinsed with MeCN (1.58 wt.).

The wet cake was charged into the reactor at 20-30° C. MeCN (15.8 wt.) was charged into the reactor at 20-30° C. Water (2.0 wt.) was then introduced into the reactor at 20-30° C. The temperature was adjusted to 20-30° C., and the reaction mixture was agitated for 1-2 hours.

The organic phase was concentrated to 4-6 volumes below 45° C. under vacuum. MeCN (15.8 wt.) was charged into the reactor, and the mixture was again concentrated to 4-6 volumes below 45° C. under vacuum. This step was repeated by charging MeCN (15.8 wt.) and concentrating to 4-6 volumes below 45° C. under vacuum. Finally, MeCN (11.8 wt.) was charged into the reactor. The reaction mixture was agitated for 30-60 minutes.

The mixture was then centrifuged, and the filter cake was rinsed with MeCN (1.58 wt.).

The wet cake was charged into the reactor. MeCN (8.7 wt.) was charged into the reactor, followed by water (0.22 wt.). The mixture was agitated for 20-22 hours at 17-22° C.

The mixture was centrifuged, and the filter cake was rinsed with MeCN (1.58 wt.).

The wet cake was dried at 35-45° C. for 16-20 hours. 2-((2-(2-oxopiperidin-1-yl)ethylcarbamoyl)methylthio) acetic acid (III)) was obtained in a yield of 79% and a purity of 99.8%.

1. A method of synthesizing an N-terminal conjugated peptidyl compound of formula (I): 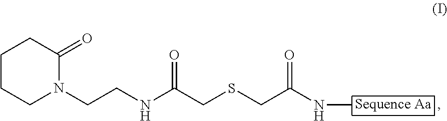 [SMILES: CNC(=O)CSCC(=O)NCCN1CCCCC1=O]
wherein Sequence Aa is a peptide, the method comprising the steps of
(i) treating a resin-bound peptide of formula (II): 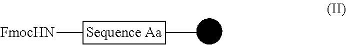
with 10 to 30% piperidine in DMF, and
(ii) reacting the product of step (i) with 2-((2-oxo-2-((2-(2-oxopiperidin-1-yl)ethyl)amino)ethyl)thio)acetic acid (III): 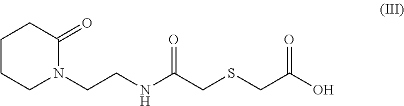 [SMILES: O=C(O)CSCC(=O)NCCN1CCCCC1=O]
under amide bond-forming conditions, wherein the amide bond-forming conditions comprise use of 2-(1H-Benzotriazole-1-yl)-1,1,3,3-tetramethylaminium tetrafluoroborate (TBTU).

2. A method of synthesizing an N-terminal conjugated peptidyl compound of formula (I): 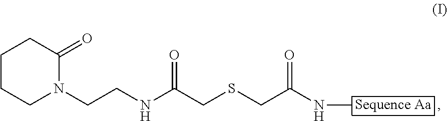 [SMILES: CNC(=O)CSCC(=O)NCCN1CCCCC1=O]
wherein Sequence Aa is a peptide, the method comprising the steps of
(i) treating a resin-bound peptide of formula (II): 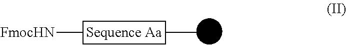
with 10 to 30% piperidine in DMF, and
(ii) reacting the product of step (i) with 2-((2-oxo-2-((2-(2-oxopiperidin-1-yl)ethyl)amino)ethyl)thio)acetic acid (III): 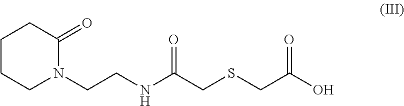 [SMILES: O=C(O)CSCC(=O)NCCN1CCCCC1=O]
under amide bond-forming conditions, wherein the amide bond-forming conditions comprise use of the combination of ethyl cyano (hydroxyimino) acetate and N,N′-diisopropylcarbodiimide (DIC).

3. The method of claim 1 or 2, wherein step (i) comprises treating the resin-bound peptide of formula (II) with 20% piperidine in DMF.

4. The method of any one of claims 1-3, wherein Sequence Aa comprises the formula W—R⁵, wherein W is a peptide sequence and R⁵ is conjugated to the C-terminus of W, wherein
R⁵ is a C-terminal amino acid amide or a C-terminal amino acid that is optionally substituted with 1 or 2 modifying groups selected from an acyl group and a PEG group
and wherein W comprises the following sequence:
EGT(Xaa4)(Xaa5)SD(Xaa8)S(Xaa10)(Xaa11)(Xaa12)(Xaa13)(Xaa14)(Xaa15)(Xaa16)(Xaa17)(Xaa18)(Xaa19)(Xaa20)(Xaa21)(Xaa22)WL(Xaa25)(Xaa26)(Xaa27)GPSSGAPPP(Xaa37)(SEQ ID NO:1); wherein:
Xaa4 is F;
Xaa5 is T or I;
Xaa8 is Y, V, L, or K*;
Xaa10 is I or S;
Xaa11 is Y, Y*, Q, A, or (Aib);
Xaa12 is L, M, or L*;
Xaa13 is D or E;
Xaa14 is K, G, R, or E;
Xaa15 is Q or I;
Xaa16 is A, H, or R;
Xaa17 is A, Q, or V;
Xaa18 is A, (Aib), K*, K, or Q;
Xaa19 is A, D, E, (Aib), or L;
Xaa20 is F or A;
Xaa21 is V or I;
Xaa22 is N, A, Q, K*, or E;
Xaa25 is I, L or V;
Xaa26 is A, K, or I;
Xaa27 is Q-R, G-R-G-K* (SEQ ID NO: 24), Q, or G; and
Xaa37 is S or absent.

5. The method of any one of claims 1-3, wherein W comprises the following sequence:
(SEQ ID NO: 2)
EGTFTSDYSIYLDKQAA(Aib)EFVNWLLAGGPSSGAPPPS.

6. The method of any one of claims 1-5, wherein R⁵ is a C-terminal lysyl amide residue that is optionally substituted with 1 or 2 modifying groups selected from an acyl group and a PEG group.

7. The method of claim 6, wherein R⁵ comprises formula (IV): 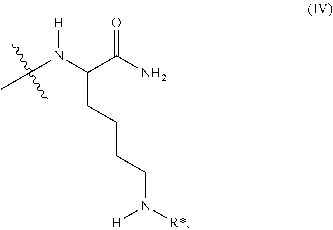
and wherein R* comprises the structure (V):

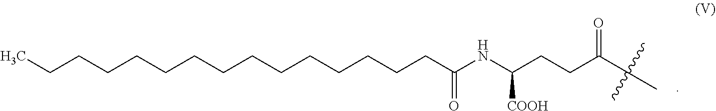 [SMILES: *C(=O)CC[C@H](NC(=O)CCCCCCCCCCCCCCC)C(=O)O]

8. The method of any one of claims 4-7, wherein W—R⁵ comprises the structure (XXII):

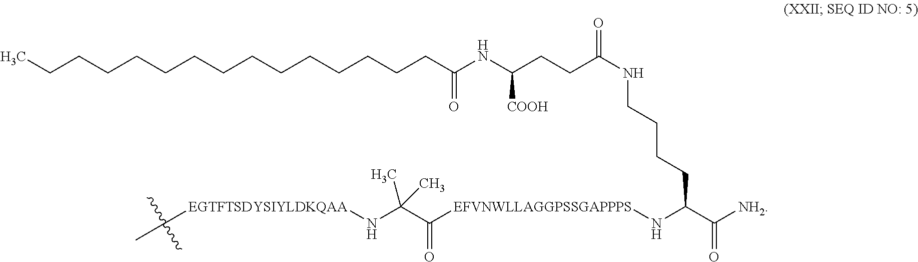

9. A method of synthesizing a compound of formula (VI): 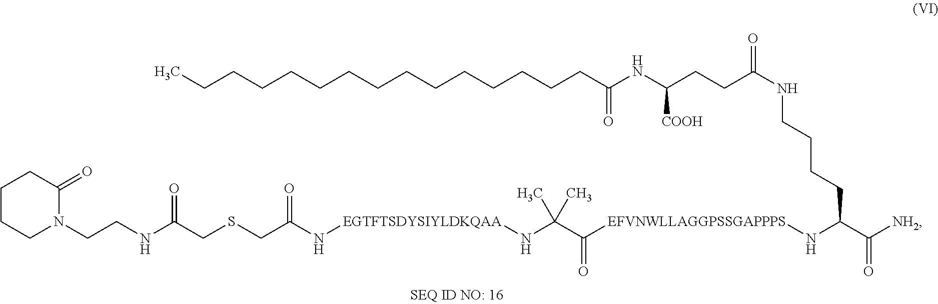
the method comprising the steps of
(i) treating a resin-bound peptide of formula (VII): 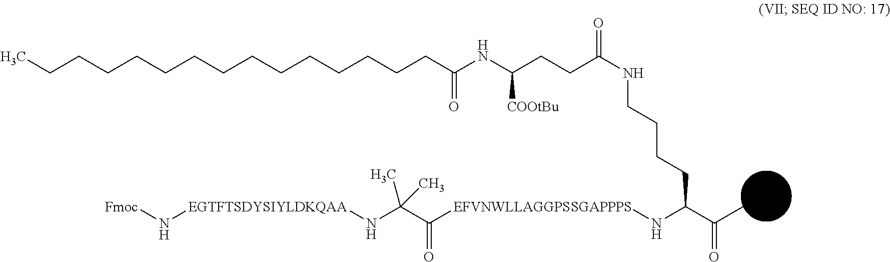
with 10 to 30% piperidine in DMF, and
(ii) reacting the product of step (i) with 2-((2-oxo-2-((2-(2-oxopiperidin-1-yl)ethyl)amino)ethyl)thio)acetic acid (III): 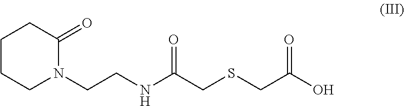 [SMILES: O=C(O)CSCC(=O)NCCN1CCCCC1=O]
under amide bond-forming conditions, wherein the amide bond-forming conditions comprise use of 2-(1H-Benzotriazole-1-yl)-1,1,3,3-tetramethylaminium tetrafluoroborate (TBTU).

10. A method of synthesizing a compound of formula (VI): 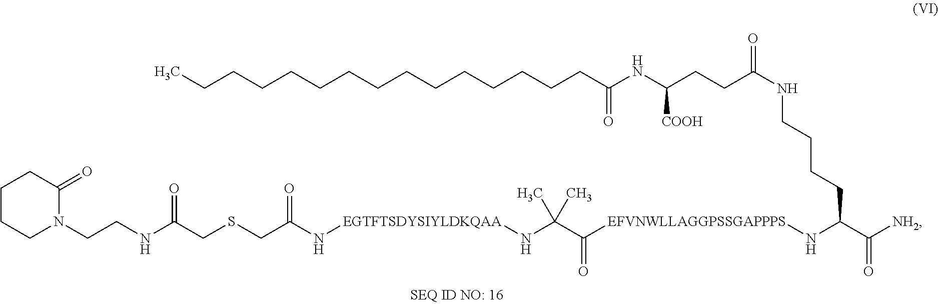
the method comprising the steps of
(i) treating a resin-bound peptide of formula (VII): 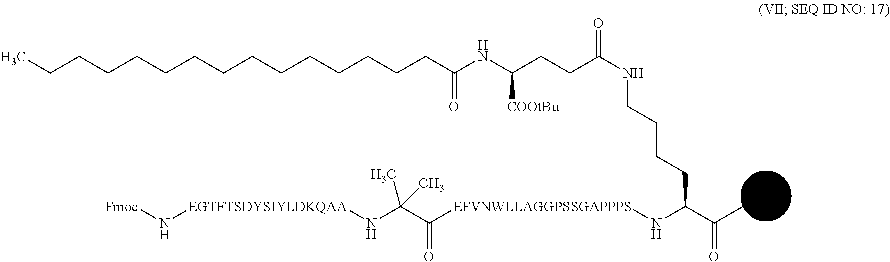
with 10 to 30% piperidine in DMF, and
(ii) reacting the product of step (i) with 2-((2-oxo-2-((2-(2-oxopiperidin-1-yl)ethyl)amino)ethyl)thio)acetic acid (I): 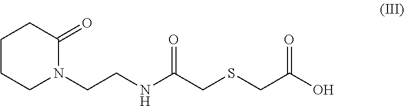 [SMILES: O=C(O)CSCC(=O)NCCN1CCCCC1=O]
under amide bond-forming conditions, wherein the amide bond-forming conditions comprise use of the combination of ethyl cyano (hydroxyimino) acetate and N,N′-diisopropylcarbodiimide (DIC).

11. The method of claim 9 or 10, wherein step (i) comprises treating the resin-bound peptide of formula (VII) with 20% piperidine in DMF.

12. The method of any one of claims 1 to 11, wherein the resin is tricyclic amide linker resin.

13. The method of any one of claims 1-12, wherein the generation of the resin-bound peptide of formula (II) comprises the steps of:
(a) treating a resin-bound amine of formula (VIII): 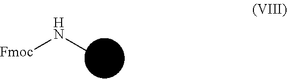
with 10 to 30%, optionally 20%, piperidine in DMF, and
(b) reacting the product of step (a) with Fmoc-Lys(Palmitoyl-Glu-OtBu)-OH (IX): 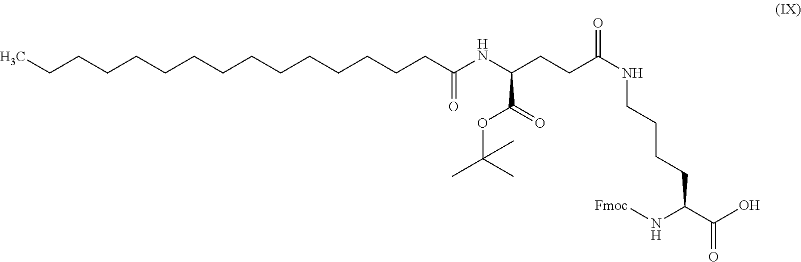
under amide bond-forming conditions, optionally wherein the amide bond-forming conditions comprise use of ethyl cyano (hydroxyimino) acetate and N,N′-Diisopropylcarbodiimide (DIC).

14. The method of any one of claims 1-13, wherein the generation of the resin-bound peptide of formula (II) comprises the steps of:
(c) treating a resin-bound peptide of formula (X): 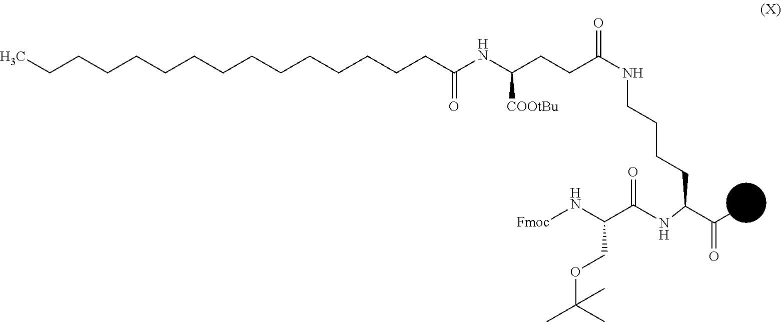
with 10 to 30%, optionally 20%, piperidine in DMF, and
(d) reacting the product of step (c) with Fmoc-Pro-Pro-OH (XI): 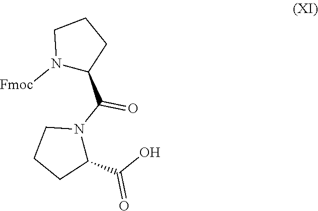
under amide bond-forming conditions, optionally wherein the amide bond-forming conditions comprise use of ethyl cyano (hydroxyimino) acetate and N,N′-Diisopropylcarbodiimide (DIC).

15. The method of any one of claims 1-14, wherein the generation of the resin-bound peptide of formula (II) comprises the steps of:
(e) treating a resin-bound peptide of formula (XII): 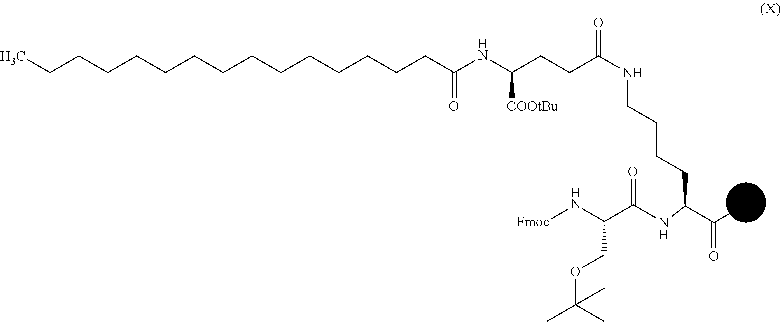
with 10 to 30%, optionally 20%, piperidine in DMF, and
(f) reacting the product of step (e) with Fmoc-Ser(tBu)-Gly-OH (XII): 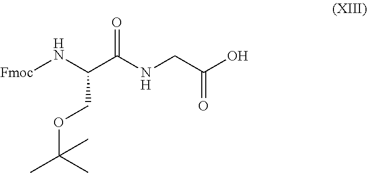
under amide bond-forming conditions, optionally wherein the amide bond-forming conditions comprise use of ethyl cyano (hydroxyimino) acetate and N,N′-Diisopropylcarbodiimide (DIC).

16. The method of any one of claims 1-15, wherein the generation of the resin-bound peptide of formula (II) comprises the steps of:
(g) treating a resin-bound peptide of formula (XIV): 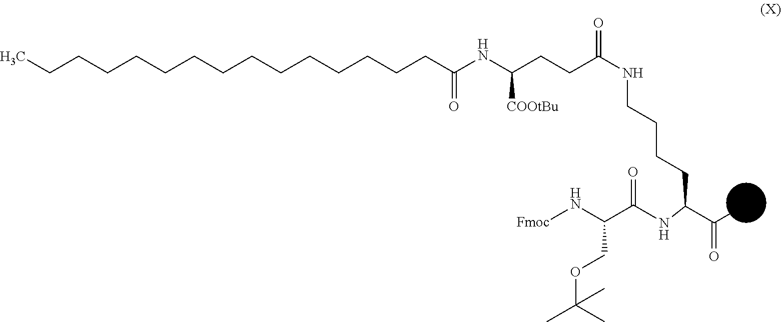
with 10 to 30%, optionally 20%, piperidine in DMF, and
(h) reacting the product of step (g) with Fmoc-Gly-Gly-OH (XV): 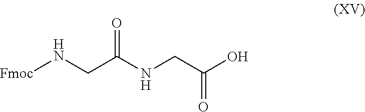
under amide bond-forming conditions, optionally wherein the amide bond-forming conditions comprise use of ethyl cyano (hydroxyimino) acetate and N,N′-Diisopropylcarbodiimide (DIC).

17. The method of any one of claims 1-16, wherein the generation of the resin-bound peptide of formula (II) comprises the steps of:
(i) treating a resin-bound peptide of formula (XVI): 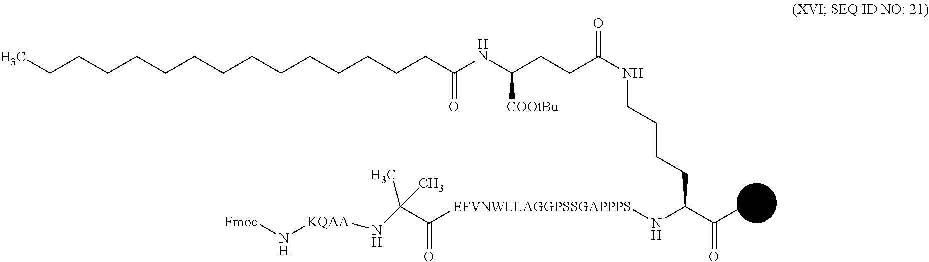
with 10 to 30%, optionally 20%, piperidine in DMF, and
(j) reacting the product of step (i) with Fmoc-Asp(OMpe)-OH (XVII): 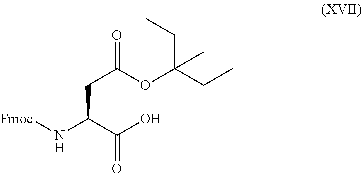
under amide bond-forming conditions, optionally wherein the amide bond-forming conditions comprise use of ethyl cyano (hydroxyimino) acetate and N,N′-Diisopropylcarbodiimide (DIC).

18. The method of any one of claims 1-17, wherein the generation of the resin-bound peptide of formula (II) comprises the steps of:
(k) treating a resin-bound peptide of formula (XVIII): 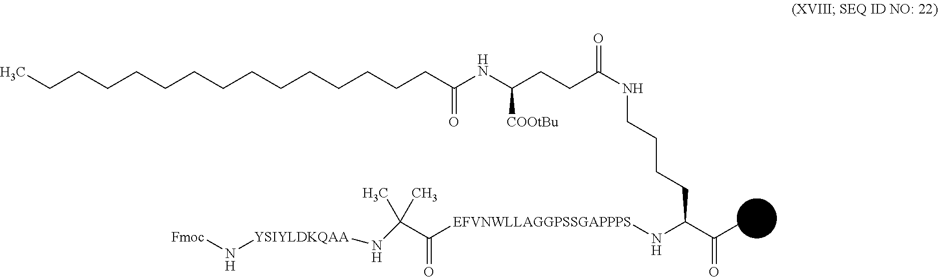
with 10 to 30%, optionally 20%, piperidine in DMF, and
(l) reacting the product of step (k) with Fmoc-Asp(OMpe)-OH (XVII): 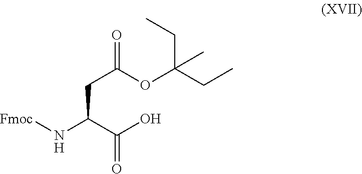
under amide bond-forming conditions, optionally wherein the amide bond-forming conditions comprise use of ethyl cyano (hydroxyimino) acetate and N,N′-Diisopropylcarbodiimide (DIC).

19. The method of any one of claims 1-18, wherein the generation of the resin-bound peptide of formula (II) comprises the steps of:
(m) treating a resin-bound peptide of formula (XIX): 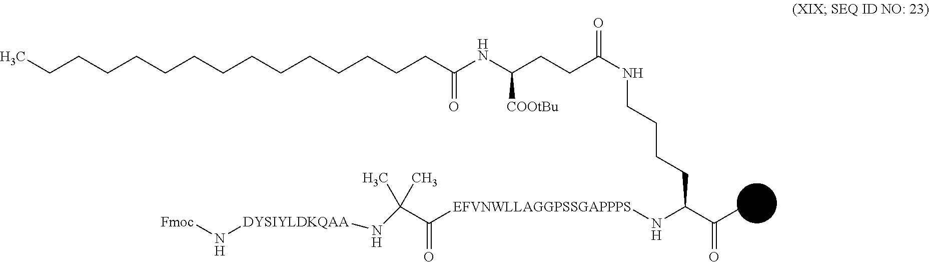
with 10 to 30%, optionally 20%, piperidine in DMF, and
(n) reacting the product of step (m) with Fmoc-Thr(tBu)-SerΨ(Me,Me)pro)-OH (XX): 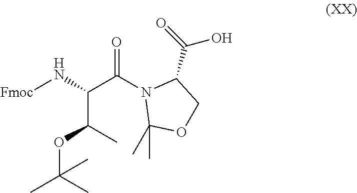
under amide bond-forming conditions, optionally wherein the amide bond-forming conditions comprise use of ethyl cyano (hydroxyimino) acetate and N,N′-Diisopropylcarbodiimide (DIC).

20. The method of any one of claims 1-19, further comprising the steps of cleaving the peptide from the resin, wherein the cleaving comprises treating the resin-bound peptide of formula (XXXII): 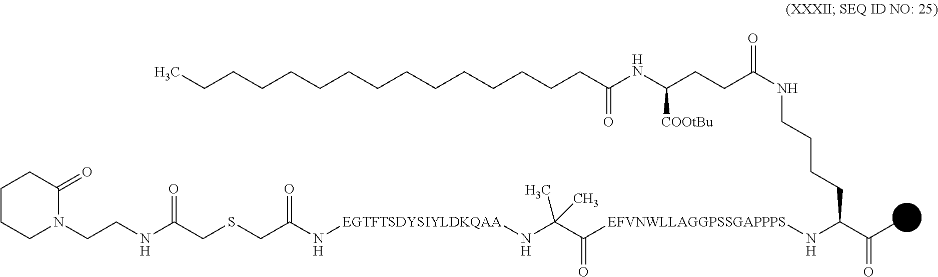
with a cleavage cocktail, optionally wherein the cleavage cocktail comprises trifluoroacetic acid (TFA) and a scavenger.

21. The method of any one of claims 1-20, further comprising the step of purifying the peptide.

22. The method of claim 21, wherein the purifying step comprises preparative liquid chromatography, tangential flow filtration, ion exchange, lyophilization, or a combination thereof.

23. The method of claim 21 or 22, wherein the purifying step comprises preparative liquid chromatography using ammonium acetate and acetonitrile as mobile phase.

24. The method of any one of claims 21-23, wherein the purifying step comprises tangential flow filtration, ion exchange into a sodium salt by addition of 1M Na₂CO₃ solution, lyophilization, or a combination thereof.

25. The method any one of claims 21-25, wherein the purifying step comprises column chromatography and a triethylammonium phosphate (TEAP) mobile phase at pH 5.4.

26. The method of any one of claims 21-26, wherein the purifying step comprises use of column chromatography and a 0.1% TFA mobile phase.

27. An N-terminal conjugated peptidyl compound of Formula (I) made by the method of any one of claims 1-26.

28. An N-terminal conjugated peptidyl compound of Formula (VI) made by the method of any one of claims 1-26.

29. A composition comprising CT-868 or the compound of claim 27 or 28 in 0.1 M NH₄HCO₃ pH 10, optionally wherein CT-868 is at a concentration of 15 g/L or 20 g/L.

30. A method of preparing the composition of claim 29, comprising dissolving lyophilized CT-868 ammonium salt in 0.1 M NH₄HCO₃ pH 10.


SEQUENCE LISTING (26 sequences)

SEQ ID NO: 1 - protein, 40 aa, synthetic construct
  VARIANT 5: note = T or I
  VARIANT 8: note = Y, V, L, or Lysine residue may be substituted with a modifying group, a C-terminal amino acid, an amino acid ester or amino acid amide thereof
  VARIANT 10: note = I or S
  VARIANT 11: note = Y, 2-amino-3-(4-hydroxyphenyl)-2-methylpropanoic acid, Q, A, or 2-aminoisobutyric acid
  VARIANT 12: note = L, M, or 2-amino-2-methylpentanoic acid, C-terminal amino acid or an amino acid ester or an amino acid amide thereof
  VARIANT 13: note = D or E
  VARIANT 14: note = K, G, R, or E
  VARIANT 15: note = Q or I
  VARIANT 16: note = A, H, or R
  VARIANT 17: note = A, Q, or V
  VARIANT 18: note = A, 2-aminoisobutyric acid, Lysine residue may be substituted with a modifying group, a C-terminal amino acid, an amino acid ester or amino acid amide thereof, K, or Q
  VARIANT 19: note = A, D, E, 2-aminoisobutyric acid, or L
  VARIANT 20: note = F or A
  VARIANT 21: note = V or I
  VARIANT 22: note = N, A, Q, Lysine residue may be substituted with a modifying group, a C-terminal amino acid, an amino acid ester or amino acid amide thereof, or E
  VARIANT 25: note = I, L or V
  VARIANT 26: note = A, K, or I
  VARIANT 27..30: note = This region may encompass one of the following sequences: QR or GRGK, wherein the Lysine residue may be substituted with a modifying group, a C-terminal amino acid, an amino acid ester or amino acid amide thereof or Q or G
  VARIANT 40: note = S or absent
EGTFXSDXSXXXXXXXXXXXXXWLXXXXXXGPSSGAPPPX

SEQ ID NO: 2 - protein, 37 aa, synthetic construct
  SITE 18: note = 2-aminoisobutyric acid
EGTFTSDYSIYLDKQAAXEFVNWLLAGGPSSGAPPPS

SEQ ID NO: 3 - protein, 37 aa, synthetic construct
  VARIANT 5: note = T or I
  VARIANT 8: note = Y, V, or L
  VARIANT 10: note = I or S
  VARIANT 11: note = Y, Q, or A
  VARIANT 12: note = L, M, or 2-amino-2-methylpentanoic acid, C-terminal amino acid or an amino acid ester or an amino acid amide thereof
  VARIANT 13: note = D or E
  VARIANT 14: note = K, G, or E
  VARIANT 17: note = A or V
  VARIANT 18: note = 2-aminoisobutyric acid or K
  VARIANT 19: note = E or L
  VARIANT 21: note = V or I
  VARIANT 22: note = N, A, or E
  VARIANT 25: note = L or V
  VARIANT 26: note = A or K
EGTFXSDXSXXXXXQAXXXFXXWLXXGGPSSGAPPPS

SEQ ID NO: 4 - protein, 37 aa, synthetic construct; identical to SEQ ID NO: 3
  VARIANT 5: note = T or I
  VARIANT 8: note = Y, V, or L
  VARIANT 10: note = I or S
  VARIANT 11: note = Y, Q, or A
  VARIANT 12: note = L, M, or 2-amino-2-methylpentanoic acid, C-terminal amino acid or an amino acid ester or an amino acid amide thereof
  VARIANT 13: note = D or E
  VARIANT 14: note = K, G, or E
  VARIANT 17: note = A or V
  SITE 18: note = 2-aminoisobutyric acid
  VARIANT 19: note = E or L
  VARIANT 21: note = V or I
  VARIANT 22: note = N, A, or E
  VARIANT 25: note = L or V
  VARIANT 26: note = A or K

SEQ ID NO: 5 - protein, 38 aa, synthetic construct
  SITE 18: note = 2-aminoisobutyric acid
  SITE 38: note = N6-[N-(1-oxohexadecyl)-L-gamma-glutamyl]-L-lysinami de
EGTFTSDYSIYLDKQAAXEFVNWLLAGGPSSGAPPPSK

SEQ ID NO: 6 - protein, 37 aa, synthetic construct
  SITE 17: note = 2-aminoisobutyric acid
  SITE 37: note = N6-[N-(1-oxohexadecyl)-L-gamma-glutamyl]-L-lysinami de
GTFTSDYSIYLDKQAAXEFVNWLLAGGPSSGAPPPSK

SEQ ID NO: 7 - protein, 37 aa, synthetic construct; identical to SEQ ID NO: 6
  SITE 17: note = 2-aminoisobutyric acid
  SITE 37: note = N6-[N-(1-oxohexadecyl)-L-gamma-glutamyl]-L-lysinami de

SEQ ID NO: 8 - protein, 37 aa, synthetic construct; identical to SEQ ID NO: 6
  SITE 17: note = 2-aminoisobutyric acid
  SITE 37: note = N6-[N-(1-oxohexadecyl)-L-gamma-glutamyl]-L-lysinami de

SEQ ID NO: 9 - protein, 37 aa, synthetic construct; identical to SEQ ID NO: 6
  SITE 17: note = 2-aminoisobutyric acid
  SITE 37: note = N6-[N-(1-oxohexadecyl)-L-gamma-glutamyl]-L-lysinami de

SEQ ID NO: 10 - protein, 37 aa, synthetic construct; identical to SEQ ID NO: 6
  SITE 17: note = 2-aminoisobutyric acid
  SITE 37: note = N6-[N-(1-oxohexadecyl)-L-gamma-glutamyl]-L-lysinami de

SEQ ID NO: 11 - protein, 37 aa, synthetic construct; identical to SEQ ID NO: 6
  SITE 17: note = 2-aminoisobutyric acid
  SITE 37: note = N6-[N-(1-oxohexadecyl)-L-gamma-glutamyl]-L-lysinami de

SEQ ID NO: 12 - protein, 37 aa, synthetic construct; identical to SEQ ID NO: 6
  SITE 17: note = 2-aminoisobutyric acid
  SITE 37: note = N6-[N-(1-oxohexadecyl)-L-gamma-glutamyl]-L-lysinami de

SEQ ID NO: 13 - protein, 37 aa, synthetic construct; identical to SEQ ID NO: 6
  SITE 17: note = 2-aminoisobutyric acid
  SITE 37: note = N6-[N-(1-oxohexadecyl)-L-gamma-glutamyl]-L-lysinami de

SEQ ID NO: 14 - protein, 38 aa, synthetic construct
  SITE 11: note = (S)-2-amino-3-(4-hydroxyphenyl)-2-methylpropanoic acid
  SITE 18: note = 2-aminoisobutyric acid
  SITE 38: note = N6-[N-(1-oxohexadecyl)-L-gamma-glutamyl]-L-lysinami de
EGTFTSDYSIXLDKQAAXEFVNWLLAGGPSSGAPPPSK

SEQ ID NO: 15 - protein, 37 aa, synthetic construct; identical to SEQ ID NO: 6
  SITE 17: note = 2-aminoisobutyric acid
  SITE 37: note = N6-[N-(1-oxohexadecyl)-L-gamma-glutamyl]-L-lysinami de

SEQ ID NO: 16 - protein, 38 aa, synthetic construct; identical to SEQ ID NO: 5
  SITE 1: note = 2-((2-oxo-2-((2-(2-oxopiperidin-1-yl)ethyl)amino)et hyl)thio)acetic acid modified residue
  SITE 18: note = 2-aminoisobutyric acid
  SITE 38: note = N6-[N-(1-oxohexadecyl)-L-gamma-glutamyl]-L-lysinami de

SEQ ID NO: 17 - protein, 38 aa, synthetic construct; identical to SEQ ID NO: 5
  SITE 18: note = 2-aminoisobutyric acid
  SITE 38: note = Lysine attached to tert-butyl N5-((S)-5,6-diamino-6-oxohexyl)-N2-palmitoyl-L-glutaminate via the side chain

SEQ ID NO: 18 - protein, 38 aa, synthetic construct; identical to SEQ ID NO: 5
  SITE 18: note = 2-aminoisobutyric acid
  SITE 38: note = N6-[N-(1-oxohexadecyl)-L-gamma-glutamyl]-L-lysinami de

SEQ ID NO: 19 - protein, 6 aa, synthetic construct
  SITE 6: note = Lysine attached to tert-butyl N5-((S)-5,6-diamino-6-oxohexyl)-N2-palmitoyl-L-glutaminate via the side chain
APPPSK

SEQ ID NO: 20 - protein, 10 aa, synthetic construct
  SITE 10: note = Lysine attached to tert-butyl N5-((S)-5,6-diamino-6-oxohexyl)-N2-palmitoyl-L-glutaminate via the side chain
PSSGAPPPSK

SEQ ID NO: 21 - protein, 25 aa, synthetic construct
  SITE 5: note = 2-aminoisobutyric acid
  SITE 25: note = Lysine attached to tert-butyl N5-((S)-5,6-diamino-6-oxohexyl)-N2-palmitoyl-L-glutaminate via the side chain
KQAAXEFVNWLLAGGPSSGAPPPSK

SEQ ID NO: 22 - protein, 31 aa, synthetic construct
  SITE 11: note = 2-aminoisobutyric acid
  SITE 31: note = Lysine attached to tert-butyl N5-((S)-5,6-diamino-6-oxohexyl)-N2-palmitoyl-L-glutaminate via the side chain
YSIYLDKQAAXEFVNWLLAGGPSSGAPPPSK

SEQ ID NO: 23 - protein, 32 aa, synthetic construct
  SITE 12: note = 2-aminoisobutyric acid
  SITE 32: note = Lysine attached to tert-butyl N5-((S)-5,6-diamino-6-oxohexyl)-N2-palmitoyl-L-glutaminate via the side chain
DYSIYLDKQAAXEFVNWLLAGGPSSGAPPPSK

SEQ ID NO: 24 - protein, 4 aa, synthetic construct
  VARIANT 4: note = Lysine residue may be substituted with a modifying group, a C-terminal amino acid, an amino acid ester or amino acid amide thereof
GRGK

SEQ ID NO: 25 - protein, 38 aa, synthetic construct; identical to SEQ ID NO: 5
  SITE 1: note = 2-((2-oxo-2-((2-(2-oxopiperidin-1-yl)ethyl)amino)et hyl)thio)acetic acid modified residue
  SITE 18: note = 2-aminoisobutyric acid
  SITE 38: note = Lysine attached to tert-butyl N5-((S)-5,6-diamino-6-oxohexyl)-N2-palmitoyl-L-glutaminate via the side chain

SEQ ID NO: 26 - protein, 38 aa, synthetic construct; identical to SEQ ID NO: 5
  SITE 18: note = 2-aminoisobutyric acid
  SITE 38: note = Lysine attached to tert-butyl N5-((S)-5,6-diamino-6-oxohexyl)-N2-palmitoyl-L-glutaminate via the side chain